(19) United States
(12) Patent Application Publication
(10) Pub. No.: US 2010/0267825 A1
(43) Pub. Date: Oct. 21, 2010

(54) TREATMENT OF SKIN DAMAGE

(75) Inventor: Bernard Malfroy-Camine, Arlington, MA (US)

Correspondence Address: NUTTER MCCLENNEN & FISH LLP, SEAPORT WEST, 155 SEAPORT BOULEVARD, BOSTON, MA, 02210-2604, US

(73) Assignee: Eukarion, Inc., Arlington, MA (US)

(21) Appl. No.: 12/759,970

(22) Filed: Apr. 14, 2010

Related U.S. Application Data
(60) Provisional application No. 61/169,682, filed on Apr. 15, 2009.

(51) Int. Cl. A61K 31/28; C12Q 1/02; C12M 1/34; A61P 17/00

(52) U.S. Cl. 514/492; 435/29; 435/287.1

(57) ABSTRACT

Methods and apparatus are disclosed for diagnosing and treating oxidative skin damage in a subject. The therapeutic method can comprise: (i) diagnosing a level of oxidative skin damage in a sample comprising stratum corneum of the subject; and (ii) recommending a therapeutic regime for treatment of oxidative skin damage in the subject, wherein said recommendation comprises a recommendation to administer a pharmaceutical formulation comprising an amount of one or more specific synthetic SOD/catalase mimetics sufficient to treat the level of oxidative skin damage of the subject as diagnosed. The diagnostic method can further include obtaining a sample from the stratum corneum of a subject; measuring the level of at least one oxidized substance in the sample; and comparing a detected level of the oxidized substance with a standard, whereby an elevated level of the oxidized substance is indicative of skin damage.

### RELATED APPLICATIONS

This application claims priority from U.S. Provisional Application Ser. No. 61/169,682, filed Apr. 15, 2009, entitled “Method of Treating Skin Damage,” which is hereby incorporated by reference in its entirety.

### FIELD

Provided are methods of diagnosis of skin damage associated with oxidative damage of the skin and providing a tailored composition and regimen for treating skin damage based on the diagnosis.

### BACKGROUND

Skin, the largest human body organ, provides a major interface between the environment and the body. The human skin is organised in multiple layers. The epidermis is the outer layer of skin, which contains 5 layers. From bottom to top, the layers are named: stratum basale; stratum spinosum; stratum granulosum; stratum licidum; and stratum corneum. The bottom layer, the stratum basale, has cells that are shaped like columns. In this layer, the cells divide and push already formed cells into higher layers. As the cells move into the higher layers, they flatten and eventually die. The stratum corneum is a multi-layered brick and mortar like structure. It consists of lipid bilayers with alternating hydrophilic and hydrophobic areas. The dermis lies beneath the epidermis and is composed of three types of tissue that are present throughout—not in layers. The types of tissue are: collagen; elastic tissue; reticular fibers. The subcutaneous tissue, which lies below the dermis, is a layer of fat and connective tissue that houses larger blood vessels and nerves. This layer is important for the regulation of temperature of the skin itself and the body.

The skin is a complicated structure with many functions. If any of the structures in the skin are not working properly, this can lead to skin damage. Oxygen, although essential for aerobic metabolism, can be converted to poisonous metabolites, such as superoxide anion and hydrogen peroxide, collectively known as reactive oxygen species (ROS). Excessive concentrations of various forms of oxygen and of free radicals can have serious adverse effects on skin causing oxidative damage. ROS can damage DNA, RNA, and proteins, including the peroxidation of membrane lipid. In particular, one major contributor to such oxidative damage in skin is hydrogen peroxide (H₂O₂).

The lasting exposure to oxidative stress caused by harmful environmental constituents, such as, air pollution generated by automobile and other industrial sources, UV radiation, smoke, food contaminants/additives/preservatives and drugs, cosmetic products, stress or diseases, and exposure to ionizing radiation including during oncology therapy, increases radicals in a living body, which manifests in increased oxidative damage in areas of the body, including the skin. Such oxidative damage creates wrinkles by destroying hyaluronic acid, elastin, collagen and a connective tissue of corium, contributes to the photoaging process, and causes diseases like dermatitis, pimples, acne, or skin cancer by destroying cells by oxidizing lipid in cell membranes. The long term consequence of oxidative stress in the skin is determined by the balance between the amount of exposure to sources of ROS, the individual's antioxidant defense capacity and its ability to repair oxidative damage.

To protect against oxidative damage the skin is equipped with a network of enzymatic and non-enzymatic antioxidant defense systems (Thiele, J. J. et al., Current Problems in Dermatology, 2001, 29:26-42). In normal, healthy skin there is a balance between the antioxidant enzymes superoxide dismutase (SOD), and catalase (CAT).

Superoxide dismutases (SODs) catalyze the reaction:

2.0₂⁻+2H⁺→0₂+H₂0₂

which removes superoxide and forms hydrogen peroxide. H₂0₂ is not a radical, but it is toxic to cells and may be rapidly converted to free radicals in the absence of catalase activity. Catalase neutralizes hydrogen peroxide by catalysis of the reaction:

2H₂0₂→2H₂0+0₂

Skin exposure to ionizing and UV radiation and/or xenobiotics/drugs generates ROS in excessive quantities that quickly overwhelm tissue antioxidants and other oxidant-degrading pathways.

A primary consequence of the accumulation of hydrogen peroxide in the skin is lipid peroxidation. Lipid peroxides at the human skin surface contribute to epidermal hyperplasia, collagen degradation and skin wrinkling and contributes to photoaging. Severe imbalance of hydrogen peroxide and other ROS and metabolites such as redox active quinones lead to tissue injury and may be involved in the pathogenesis of multiple skin disorders/allergic reactions/neoplasms, and radiation dermatitis.

### UVA/UVB/UVC Damage

The sun emits ultraviolet radiation in the UVA, UVB, and UVC bands, but because of absorption in the atmosphere's ozone layer, 98.7% of the ultraviolet radiation that reaches the Earth's surface is UVA. UVA, UVB and UVC can all damage collagen fibers and thereby accelerate aging of the skin. UVA was considered less harmful, but is now known to contribute to skin cancer via the indirect DNA damage (free radicals and reactive oxygen species). It penetrates deeply but it does not cause sunburn. UVA does not damage DNA directly like UVB and UVC, but it can generate highly reactive chemical intermediates, such as hydroxyl and oxygen radicals, which in turn can damage DNA.

The reddening of the skin due to the action of sunlight depends both on the amount of sunlight as well as the sensitivity of the skin (“erythemal action spectrum”) over the UV spectrum. UVB light can cause direct DNA damage creating “TT” dimers in the DNA. The mutations that are caused by the direct DNA damage carry a UV signature mutation that is commonly seen in skin cancers. This cancer connection is one reason for concern about ozone depletion and the ozone hole. UVB causes some damage to collagen but at a very much slower rate than UVA.

As a defense against UV radiation, the amount of melanin in the skin increases when exposed to moderate levels of radiation, depending on skin type; this is commonly known as a sun tan. The purpose of melanin is to absorb UV radiation and dissipate the energy as harmless heat, blocking the UV from damaging skin tissue. UVA gives a quick tan that lasts for days by oxidizing melanin that was already present and triggers the release of the melanin from melanocytes. UVB yields a tan that takes roughly 2 days to develop because it stimulates the body to produce more melanin. The photochemical properties of melanin make it an excellent photoprotectant. However, sunscreen chemicals cannot dissipate the energy of the excited state as efficiently as melanin.

Sunscreen prevents the direct DNA damage which causes sunburn. Most of these products contain an SPF rating to show how well they block UVB rays. The SPF rating, however, offers no data about UVA protection. Some sunscreen lotions now include compounds such as titanium dioxide which helps protect against UVA rays. Other UVA blocking compounds found in sunscreen include zinc oxide and avobenzone. Although sunscreen provides a good block to the UV radiation, it does not treat any oxidative damage from UV radiation that penetrates the epidermis.

### Radiation Damage

It is well recognized that x-ray radiation is a valuable curative and palliative oncological tool. Radiation therapy is employed for the treatment of many benign and malignant lesions and for organ and bone marrow transplantation modalities. Although the value of radiation in oncology therapy is universally recognized, the use of high energy radiation in conjunction with chemotherapy is not without its adverse side affects. For example, x-ray radiation has been shown to induce early and/or late radiation-induced skin changes such as skin erythema and ulceration including the severe skin reaction with open blisters, named “moist desquamation”, i.e., radiation dermatitis, as well as the development of skin cancers.

When tissues are exposed to ionizing radiation, gamma energy is absorbed by water contained within the cells resulting in breakage of the oxygen-hydrogen covalent bonds of the water molecule leaving hydrogen and hydroxyl radicals in situ. It is known that the hydroxyl radical is quite reactive in its interaction with other biomolecules generally thought to be responsible for setting off chain reactions including interactions with the purine and pyrimidine bases of nucleic acids. Radiation-induced cutaneous carcinogenesis may be initiated by free radical damage. Since radiation often is applied to the human body as a treatment of “deep” lesions such as lung, breast, liver and brain malignancies, it is important to protect the skin from radiation-induced skin damage.

A variety of moisturizers and lipid preparations, including aloe vera and mineral oil, have been used to protect skin from x-ray radiation in therapy. However, such compositions have enjoyed little or no success in preventing or healing radiation skin damage.

Based on the forgoing, it is clear that oxidative damage of the skin occurs in a number of situations and to varying degrees whereby skin is exposed to environmental insults, stress or diseases, or to ionizing radiation during oncology therapy and that a need exists to develop suitable therapies for skin damage associated with oxidative damage of the skin.

### SUMMARY

In one example, a method of treating oxidative skin damage in a subject is provided, said process comprising:
(i) obtaining a sample from a stratum corneum of a subject;
(ii) measuring the level of at least one oxidized substance in the sample; and
(ii) comparing a detected level of the oxidized substance with a standard, whereby an elevated level of the oxidized substance is indicative of oxidative skin damage. The method can further comprise recommending a therapeutic regime for treatment of oxidative skin damage in the subject, wherein said recommendation comprises a recommendation to administer a pharmaceutical formulation comprising an amount of one or more specific synthetic SOD/catalase mimetics sufficient to treat the level of oxidative skin damage of the subject.

As used herein, the term “oxidative damage index” is taken to mean a numerical ratio wherein the amount of an oxidized form of a substance present in a sample from the stratum corneum of the skin of a subject, e.g., ventral forearm, or face, that can be collected using a non invasive apparatus and is measured and expressed relative to the amount of the non-oxidized form of that substance measured in the same skin sample of the subject.

In one embodiment, a sample can be obtained by extracting a substance with a solvent. The substance to be extracted can be, for example, a lipid from the stratum corneum. Moreover, the solvent for extracting the substance can be an organic solvent or an alcohol based solvent. Additionally, the solvent can contain a non-catalytic antioxidant. Some non-limiting examples of an organic solvent can be acetone, and alcohol-based solvents can be ethanol, or isopropanol, and non-catalytic antioxidants can be, urate, ascorbate, α-tocopherol, or bilirubin.

In another embodiment of obtaining the sample, a non invasive collection apparatus can be used. Alternatively, the sample can be collected by contacting a region of skin with the apparatus, such that the apparatus is in fluid communication with the skin region and comprises a solvent capable of extracting lipids from the stratum corneum.

In another example, the process as described according to any example hereof may further comprise obtaining a sample comprising stratum corneum of the subject. A suitable site of stratum corneum of the subject may be any convenient skin surface e.g., a region of arm, leg, face, torso, or back. Samples can be obtained e.g., by a skilled technician, or a physician such as a dermatologist. In one example, a sample can be obtained by collecting the sample using a non invasive collection apparatus. A region of skin can be contacted with the apparatus, such that the apparatus is in fluid communication with the skin region and comprises a solvent capable of extracting a substance from the stratum corneum. In one embodiment, the substance can be lipids. In another example, skin surface lipids can be collected by an ethanol wash of the stratum corneum. An ethanol wash may be achieved e.g., using a syringe comprising ethanol and a removable membrane at an end that is contactable with and capable of being in sealing engagement with skin, wherein the removable membrane collects lipids washed from the stratum corneum by ethanol. In this example, the membrane is then analyzed directly for oxidized and non-oxidized lipid content on which the calculation of oxidative damage index is made. In another example, an ethanol wash is performed using a device exemplified by any one of FIGS. 1 to 3.

In one example, the apparatus for diagnosing a skin condition can include:
a skin contacting base member defining a reservoir adapted for fluid communication with a region of skin;
a source of solvent suitable for extracting a substance from the skin region; and
at least one channel for introducing the solvent into the reservoir when the reservoir is in fluid communication with the skin region and for withdrawing the solvent after it has extracted the substance from the skin. The skin contacting base member can also have a skin contacting member or device such as scrapers, cotton swaps, adhesive tape or a solvent carrying towelette to obtain the sample. The base member can also house a skin-contacting lip for maintaining contact without leakage from the reservoir when the based member is pressed against the skin and solvent is introduced into the chamber.

The apparatus also comprises a channel for introducing a solvent into the reservoir. The solvent source can have a releasable seal to prevent introduction of the solvent into the reservoir until the seal is released by a user. In one embodiment, the channel can serve to introduce and remove the solvent. In another embodiment, a syringe can introduce and withdraw the solvent via the channel. In yet another embodiment, the apparatus can have at least two channels, one that is adapted to introduce the solvent and the other adapted to withdraw the solvent.

In one example, the apparatus houses a solvent capable of extracting a substance from the sample. The solvent can be an organic solvent or an alcohol based solvent. Additionally, the solvent can contain a non-catalytic antioxidant. Some non-limiting examples of an organic solvent can be acetone, and alcohol-based solvents can be ethanol, or isopropanol, and non-catalytic antioxidants can be, urate, ascorbate, α-tocopherol, or bilirubin.

In another example, the apparatus can have a collection chamber for storing the extracted substance. Such an embodiment may include the apparatus configured for direct or indirect injection of the sample into a chromatography apparatus or mass spectrometer for further analysis. Alternatively, the apparatus can contain an indicator reagent that binds to one or more oxidized substances present in the sample.

In another example, the process as described according to any example hereof may further comprise measuring the level of at least one oxidized substance. The substance can be an oxidized or peroxidized lipid in the sample. Some non-limiting examples of oxidized or peroxidized lipid can be oxidized phospholipid 1-palmitoyl-2-arachidonoyl-sn-glycero-3-phosphorylcholine (OxPAPC) or peroxidized squalene (sqOOH) in the sample. Additionally, the oxidized substance in the sample can be subjected to chromatography or mass spectroscopy for further analysis.

In another example, the oxidative skin damage is oxidative damage arising e.g., from exposure to ultraviolet radiation, ionising radiation, one or more chemotherapeutic agents, or a consequence of the normal aging process.

A diagnosis of the level of oxidative skin damage may be achieved by several means. In one example, a method of diagnosing skin damage can comprise:
obtaining a sample from the stratum corneum of a subject;
measuring the level of at least one oxidized substance and at least one non-oxidized substance in the sample;
and evaluating a level of skin damage in a subject by:
(i) calculating an oxidative damage index of the sample; and
(ii) comparing the oxidative damage calculated at (i) to a baseline level of oxidative damage to skin of a healthy control subject, wherein a greater oxidative damage than baseline is indicative of the presence of skin damage in the skin of said subject.

The oxidative damage index can be expressed as a numerical ratio, such that the detected amount of an oxidized form of a substance present in the stratum corneum of any suitable part of the skin of a subject is measured and expressed relative to a detected amount of a non-oxidized form of that substance measured in the same skin sample of the subject.

For example, the oxidative damage index may be calculated by determining relative amounts of oxidized and non-oxidized lipids in a sample of the stratum corneum of the subject. For example, the oxidative damage index is calculated by the algorithm:

oxidized lipid/(non-oxidized lipid+oxidized lipid)×100

In another example, the oxidative damage index may be calculated by determining relative amounts of peroxidated lipid such as squalene and non-peroxidated lipid such as squalene. For example, the oxidative damage index is calculated by the algorithm:

peroxidated lipid/(non-peroxidated lipid+peroxidated lipid)×100

In a yet another example, the amount of peroxidated squalene (sqOOH) is measured and compared to the amount of squalene (sq) present in the sample, wherein the oxidative damage index is calculated by the algorithm:

sqOOH/(squalene+sqOOH)×100

In one more example, the oxidative damage index can be calculated by determining relative amounts of the oxidation product of phospholipid 1-palmitoyl-2-arachidonoyl-sn-glycero-3-phosphorylcholine (OxPAPC) and non-peroxidized phospholipid 1-palmitoyl-2-arachidonoyl-sn-glycero-3-phosphorylcholine (PAPC) present in the sample according to the following formula:

OxPAPC/(PAPC+OxPAPC)×100

In a further example, the process can comprise calculating an oxidative damage index for a baseline level of oxidative damage to a region of the stratum corneum of the subject where visible symptoms of skin damage are absent.

In one embodiment, the oxidative damage can be compared by a programmed computer comprising a processor and a lookup table of one or more baseline values stored in memory.

Where a subject does present with one or more visible symptoms of skin damage, a diagnosis of the level of oxidative skin damage may comprise:

(i) calculating the oxidative damage index for a region of the stratum corneum of the subject where visible symptoms of skin damage are present; and
(ii) comparing the oxidative damage calculated at (i) to a baseline level of oxidative damage to skin of a healthy control subject, wherein a greater oxidative damage than baseline is indicative of the presence of skin damage in the skin of said subject.

Alternatively, or in addition, a diagnosis of the level of oxidative skin damage may comprise:

(i) calculating the oxidative damage index for a region of the stratum corneum of the subject where symptoms of skin damage are present; and
(ii) comparing the oxidative damage calculated at (i) to a baseline level of oxidative damage to a region of the stratum corneum of the subject where visible symptoms of skin damage are absent, wherein a greater oxidative damage than baseline is indicative of the presence of skin damage in the skin of said subject.

In these examples, a baseline level of oxidative damage to a region of the stratum corneum of the subject where visible symptoms of skin damage are absent is generally represented by an oxidative damage index. The oxidative damage indices for the sample and undamaged region of the stratum, corneum are generally calculated as described according to any other example hereof

In another example, the process as described according to any example hereof may further comprise monitoring therapy e.g., to thereby determine a reduction in skin damage such as by reduced visible symptoms, or prevention of skin damage over time. Generally, such monitoring of therapy comprises diagnosing a level of oxidative skin damage before and after administration of a SOD/Catalase mimetic compound that is recommended in the process. Monitoring of therapy may also comprise diagnosing a level of oxidative skin damage during therapeutic intervention e.g., between repeated administrations of a SOD/catalase mimetic compound.

In another example, the process as described according to any example hereof may further comprise administering the pharmaceutical formulation to the subject, such as for a time and under conditions sufficient to reduce the level of skin damage associated with oxidative skin damage in the subject or to prevent further skin damage.

The method as described according to any example hereof can further comprise recommending a therapeutic regime for treatment of oxidative skin damage in the subject, wherein said recommendation comprises a recommendation to administer a pharmaceutical formulation comprising an amount of one or more specific synthetic SOD/catalase mimetics sufficient to treat the level of oxidative skin damage of the subject. The method can further comprise deliverying the pharmaceutical formulation comprising an effective amount of a synthetic SOD/catalase mimetic to the subject's skin.

As used herein, the term “recommend” or variants such as “recommendation” or “recommending” shall be taken to mean preparing, and/or providing a suitable pharmaceutical formulation as described herein according to any embodiment with instructions for use, or administering the suitable pharmaceutical formulation according to instructions provided with said pharmaceutical formulation. It will also be apparent from the disclosure herein that at least one active agent of the pharmaceutical formulation and in one embodiment a suitable concentration thereof sufficient to treat oxidative skin damage in the subject is selected on the basis of the diagnosis of the level of skin damage.

In one embodiment, the synthetic SOD/catalase mimetic can be a salen-manganese complex such as salen-Mn(III) complex having a structure according to Structure I:

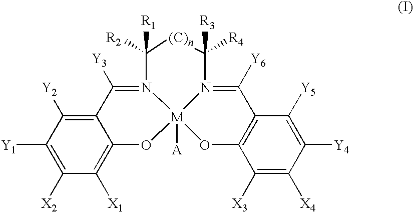

wherein M is a transition metal ion; A can be an axial ligand composed of a halide, acetate, acetyl, acetoxy, ethoxy, formate, formyl, methoxy, PF₆, triflate, tosylate, A can be an oxygen atom bound to the transition metal (M), and A can be Cl, Br, F, MeO and OAc; n can be 0, 1, 2, and 6; X₁ X₂, X₃ and X₄ can be hydrogen, silyls, aryls, arylalkyls, primary alkyls, secondary alkyls, tertiary alkyls, alkoxys, aryloxys, aminos, quaternary amines, heteroatoms, F, Cl, Br, OAc, OMe, OH, and H; Y₁, Y₂, Y₃, Y₄, Y₅, and Y₆ can be hydrogen, halides, alkyls, aryls, arylalkyls, silyl groups, aminos, aryls bearing heteroatoms, aryloxys and alkoxys; and R, R₂, R₃ and R₄ can be H, CH₃, C₂H₅, C₆H₅, O-benzyl, primary alkyls, fatty acid esters, substituted alkoxyaryls, heteroatom-bearing aromatic groups, arylalkyls, secondary alkyls, and tertiary alkyls.

Alternatively, the synthetic SOD/catalase mimetic can be a salen-manganese complex having a structure according to any of the structures of compounds C1, C4, C6, C7, C9, C10, C11, C12, C15, C17, C20, C22, C23, C25, C27, C28, C29, C30, C31-C94 as shown in Figures or the Structures X-XXII as shown in FIGS. 5D through 5I.

In another example, the synthetic SOD/catalase mimetic can be a metalloporphyrin having a structure according to any of the structures XXVI-XXXII as shown in FIG. 5K-5O.

In yet another example, the synthetic SOD/catalase mimetic can be a cyclic salen-metal compound having a structure according to any of the structures C101-C155 as shown in FIGS. 6AA-6AQ.

In one more example, the synthetic SOD/catalase mimetic can be an orally bioavailable water soluble metalloporphyrin derivative having a structure according to Structure XXXIV, Structure XXXV, Structure XXXVI as shown in FIGS. 5P-5Q or any of the compounds as shown in FIGS. 7A-7C.

The steps, features, integers, compositions and/or compounds disclosed herein or indicated in the specification of this application individually or collectively, and any and all combinations of two or more of said steps or features.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the embodiments described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

### BRIEF DESCRIPTION OF THE DRAWINGS

FIG. 1 is a longitudinal sectional view of the device prior to use.

FIG. 2 is a cross-sectional view of the distal portion of the device.

FIG. 3 is a cross-sectional view of the proximal portion of the device showing the base member when the protective member is removed.

FIG. 4 is a longitudinal sectional view of the device when obtaining a sample.

FIG. 5A through 5Q show structural formulae of SOD/catalase mimetics. Groups for all structures except for structures X-XXIV whereby M is a transition metal selected from Mn, Cu, V, Zn, Fe, Pd, Cr, Co; X₁, X₂, X₃, and X₄ are independently halide, hydrogen, alkoxy, aryloxy, hydroxy, amine, —NHCOR where R is an optionally substituted hydrocarbyl, C₆H₅, or lower alkyl; Y₁, Y₂, Y₃, and Y₄ are independently halide, hydrogen, alkoxy, aryloxy, hydroxy, amine, —NHCOR where R is an optionally substituted hydrocarbyl, C₆H₅, or lower alkyl; A is an axial ligand composed of a halide, acetate, formate, PF₆, triflate, tosylate, or is an oxygen atom bound via a double bond to the metal (M); R₁ through R are independently H, optionally substituted hydrocarbyl, CH₃, C₂H₅, C₆H₅, O-benzyl, primary alkyls, fatty acid esters, substituted alkoxyaryls, heteroatom-bearing aromatic groups, arylalkyls, secondary alkyls, or tertiary alkyls. Often, R₁ and R₃ are covalently linked together, in one embodiment by a C—C, C═C, C—O, C—N, or C═N bond, or are linked as parts of an aromatic ring (e.g., benzene ring composed of Rₓ and R₃), saturated ring, or heterocycle. Z₁, Z₂, Z₃, and Z₄ are independently selected from hydrogen, halide, lower alkoxy, and lower alkyl. Generally, the bridge structure, if present, is an optionally substituted hydrocarbyl, in one embodiment —(CH₂)n-, where n is generally 1, 2, 3, 4, 5, 6, 7 or 8, often 2 or 6, and when 6, often C(n) is a benzene ring.

FIGS. 6A-6AQ show structural formulae of useful SOD/catalase mimetics that are salen metal compounds and cyclic salen metal compounds.

FIGS. 7A-7C show structural formulae of useful SOD/catalase mimetics that are orally bioavailable water soluble metalloporphyrin derivatives.

### DETAILED DESCRIPTION

### 1. Diagnosing a Level of Oxidative Skin Damage

### 1.1 Detection of Oxidation Products in Skin Samples

It will be understood that diagnosing the level of oxidative damage in a sample comprises determining the level of oxidation of substances present in the sample of stratum corneum of a subject. The level of oxidation of such substances is used to establish a diagnosis. Suitable substances that may be assayed for oxidation state will be apparent to those skilled in the art and includes for example, lipids.

Skin lipids present in the stratum corneum that are suitable for use in the process according to any embodiment as described herein include, lipids present in sebum, e.g., produced by sebaceous glands, and those produced by the epidermis. These lipids include a diverse group of compounds, comprising triglycerides, diglycerides, ceramides, free fatty acids, wax esters, cholesterol and cholesterol esters, and squalene. The quantity and composition of the skin surface lipids differ from place to place on the body, and may to some extent be related to the number of sebaceous glands in a given area of the skin. It will be apparent to those skilled in the art that any skin lipids that are readily available on the surface of skin, e.g., on the stratum corneum may be used in the process.

A person skilled in the art will understand that triglycerides, diglycerides, ceramides, free fatty acids, wax esters, cholesterol and cholesterol esters, or squalene lipids containing double bonds, and/or susceptible to oxidation are useful in the process. Accordingly, analysis of oxidated skin lipids includes oxidated forms of triglycerides, diglycerides, ceramides, free fatty acids, wax esters, cholesterol and cholesterol esters, or squalene, e.g., cholesterol 7-hydroperoxides, oxidized skin wax esters, and peroxidated squalene.

Polyunsaturated fatty acids and cholesterol become oxidized to become lipid hydroperoxides and oxycholesterol, respectively. Oxidized lipids undergo fragmentation to form aldehydes. The level of oxidated and/or peroxidated products in a sample of stratum corneum may be measured by any means known in the art such as fluorescence methods, high performance liquid chromatography (HPLC), mass spectrometry, or specific antibodies and western blot analysis such that the level of oxidated/peroxidated products in the sample may be determined

In one example, the oxidation product of phospholipid 1-palmitoyl-2-arachidonoyl-sn-glycero-3-phosphorylcholine (PAPC) may be measured in the process. Polyunsaturated fatty acyl residues at the sn-2 position of the glycerol backbone, such as the arachidonoyl moiety in PAPC, are especially prone to oxidative modification. Thus, the oxidation of PAPC (OxPAPC) leads to the addition of oxygen atoms as well as to fragmentation of the arachidonate moiety. OxPAPC may be measured using HPLC or Mass Spectrometry essentially as described in Watson et al., (1997) Journal of Biological Chemistry 272 (21): 13957-13607.

In another example, squalene and squalene peroxide in skin samples maybe measured by any known HPLC method in the art suitable for this purpose. For example, one HPLC method comprises an on-line system for the separation of squalene and squalene peroxide from other lipids on a reversed-phase C18 column. Squalene is detected directly after column separation with a UV detector set at 220 nm. Squalene peroxide is detected on-line after a postcolumn reaction with a solution of an iron(II) salt and sulfosalicylic acid, resulting in a colored complex that can be detected with a visible light detector set at 510 nm. In order to prepare a squalene standard, 25 mg of squalene is dissolved in 10 ml ethanol in a 50-ml gauged vial with vigorous stirring and the vial filled with ethanol to 50 ml. This is mixed and diluted to 0.05% solution in ethanol 10 times, aliquot in 1-ml vials, store at −20°, and use as a standard. The squalene peroxide standard solution is prepared by dissolving in a 100-ml gauged vial 1 g of squalene in 20 ml ethanol with vigorous stirring, adding 10 ml of a 0.5 mM methylene blue solution in ethanol, filling the vial with ethanol up to 100 ml, and mixing. The resulting solution is poured into a beaker with a diameter of 10 cm and is irradiated with a dose of 1.8 J/cm2 of UVB. Methylene blue is removed by solid-phase extraction using silica cartridges (Maxi-clean silica cartridge 600 mg, Alltech) (one cartridge per 50 ml). The squalene peroxide level of this solution is determined by titration using a standardized sodium thiosulfate solution.

The ethanol-soluble, dry material obtained from the skin surface is recovered with twice 110/zl of ethanol. Ninety microliters is transferred in a vial fitting the autoinjector device of an HPLC chromatography apparatus equipped with a Nucleosil CIS, 4.6×150-mm column (Macherey-Nagel, Dtiren, Germany) in a column oven at 50°. After injecting the sample onto the system, the column is eluted with a mobile phase, starting with 90% ethanol/10% water, then gradually evolving in 5 min to 90% ethanol, 10% acetonitrile, 0.02% acetic acid and running at this condition for 15 min. Afterward the system is reconditioned with the initial mobile phase. The flow rate is 0.8 ml/min. The chromatographic system is equipped with a UV detector set at 220 nm, which allows quantification of the UV-absorbing material. Squalene elutes with a retention time of 17 min. Immediately after passing through the UV detector, the eluent is mixed with a solution of 0.04% FeSO4.7H20, 1.6% sulfosalicylic acid in methanol/water 90/10 acidified with 0.1% acetic acid via a low dead volume T switch at a rate of 0.2 ml/min. The resulting mixture passes through a postcolumn reactor (5 m×0.5 mm ID knitted PTFE tube in a column oven at 75°) where ferrous iron is oxidized by the peroxides to yield ferric iron that will react with the sulfosalicylic acid to form a purple complex. 5 This complex elutes around 13 min and can be quantified with a second UV/VIS detector set at 510 nm.

### 1.2 Oxidative Damage Index

As used herein, the term “oxidative damage index” is taken to mean a numerical ratio wherein the amount of an oxidized form of a substance present in a sample from the stratum corneum of the skin of a subject, e.g., ventral forearm, or face, that can be collected using a non invasive apparatus and is measured and expressed relative to the amount of the non-oxidized form of that substance measured in the same skin sample of the subject.

It is to be understood that “baseline level of oxidative damage” refers to the oxidative damage to skin from the same region of the body of a subject of similar race and sex as the skin being diagnosed, albeit not subjected to the effector(s) of oxidative damage. For example, the healthy control subject may be a subject that has not been exposed to a damaging level of ultraviolet radiation or ionising radiation or a chemotherapeutic agent. The healthy control subject may be a subject of an age that is insufficient to have had aging-related damage to the skin e.g., less than about 60 years of age or less than about 55 years of age or less than about 50 years of age or less than about 45 years of age or less than about 40 years of age or less than about 35 years of age or less than about 30 years of age or less than about 25 years of age or less than about 20 years of age or less than about 15 years of age.

In another example, the subject being diagnosed presents with one or more visible symptoms of skin damage e.g., a visible abnormality in the integrity of the epidermis associated with skin damage such as any one or more symptoms selected from the group consisting of: redness; burning, itching, rash, dry-flaking skin, wrinkling, freckling, dilatation of capillaries, irregular pigmentation, liver spot, sun-burn, swelling, blistering, broken skin, eczema, psoriasis, acne, urticaria, dermatitis (e.g., contact dermatitis, atopic dermatitis, seborrheic dermatitis, stasis dermatitis, seborrhoeic dermatitis, allergic contact dermatitis, perioral dermatitis, dermatitis herpetiformis, irritant contact dermatitis, or radiation dermatitis), erythema, ulceration, keratoses, basal cell carcinoma, squamous cell carcinoma, melanoma and combinations thereof.

By “visible” is meant visible to the naked eye and/or with the aid of a microscope or otherwise detectable by skilled physician such as a dermatologist.

In another example, the subject presents with one or more visible symptoms of skin damage arising from sun exposure e.g., exposure to UV radiation such as UVA, UVB or UVC radiation. In one example, sun exposure is acute, or short term, e.g., 1 to about 24 hours duration, and in some instances at least about 4 hours duration. The UV radiation can comprise UVB and/or UVC. In this case, the subject may present with sun-burn, redness, burning, itching, rash, dry-flaking skin, or skin erythema.

In another example, sun exposure is longer term or long-term sun exposure, e.g., one or more exposures of greater than 24 hours duration. In some instances, long-term sun exposure comprises exposure to UVA radiation e.g., one or more exposures to UVA radiation of greater than 24 hours duration per exposure. In this case, the subject may present with wrinkles, one or more symptoms of photoaging including freckling, dilatation of capillaries and irregular pigmentation (liver spots) and or one or more symptoms of skin cancer such as those associated with basal cell carcinoma, squamous cell carcinoma, or melanoma, or combinations thereof.

For example, the subject may present with one or more visible symptoms of skin damage arising from exposure to an environmental pollutant, including but not limited to cigarette smoke, or air pollution generated by automobile and other industrial sources, including photochemical smog e.g., due to presence of nitrous oxide or nitric oxide. In this case, the subject may present with wrinkles, one or more symptoms of photochemical aging including freckling, dilatation of capillaries and irregular pigmentation (liver spots) and any symptoms of skin cancer such as those associated with basal cell carcinoma, squamous cell carcinoma, or melanoma.

For example, the subject may present with one or more visible symptoms of skin damage arising from exposure to inflammatory agent(s) and/or allergen(s). In this case, the subject may present with redness, itchiness, urticaria, eczema, psoriasis, acne, or dermatitis.

For example, the subject may present with one or more visible symptoms of skin damage arising from exposure to ionizing radiation. In this case, the subject may present with radiation dermatitis, skin erythema, ulceration, open blisters, moist desquamation, or a symptom of skin cancer such as associated with basal cell carcinoma, squamous cell carcinoma, or melanoma.

For example, the subject may present with one or more visible symptoms of skin damage arising from exposure to food contaminant(s) and/or food additive(s) and/or food preservative(s) and/or medicinal drug(s) and/or drug(s) of addiction and/or topical formulation such as a cosmetic product and/or stress(es) and/or disease(s).

Whilst it is understood that the diagnosis of the level of skin damage in a sample may be achieved by any means that comprises determining the level of oxidated substances in a sample according to any embodiment as described herein, one example comprises calculating the oxidative damage index of a sample of stratum corneum of the subject.

For example, the oxidative damage index is calculated by determining the relative amounts of oxidized and non-oxidized lipids in a sample of stratum corneum of a subject by any means described in the art or as described and/or exemplified herein. It will also be understood that measuring the oxidative damage index comprises measuring an oxidated form of a lipid obtained from a sample of the stratum corneum, as described herein, and expressed relative to a standard, such as the total (oxidated form plus non-oxidated form) of that lipid in the sample. Accordingly, the measurement of the oxidative damage index is not limited to a region of skin, and is not affected by the total amount of lipid in the skin, as this varies from site to site.

For example, the oxidative damage index may be calculated by determining relative amounts of oxidized and non-oxidized lipids in a sample of the stratum corneum of the subject. For example, the oxidative damage index is calculated by the algorithm:

oxidized lipid/(non-oxidized lipid+oxidized lipid)×100

In another example, the oxidative damage index may be calculated by determining relative amounts of peroxidated lipid such as squalene and non-peroxidated lipid such as squalene. For example, the oxidative damage index is calculated by the algorithm:

peroxidated lipid/(non-peroxidated lipid+peroxidated lipid)×100

In one example, the amount of peroxidated squalene (sqOOH) is measured and compared to the amount of squalene (sq) present in the sample, wherein the oxidative damage index is calculated by the algorithm:

sqOOH/(squalene+sqOOH)×100

### 1.3 Comparing to Baseline Levels

It will be understood that the process comprises determining the level of oxidative damage in a sample of stratum corneum in a subject according to any embodiment herein and comparing that level to a baseline level. It is to be understood that “baseline level of oxidative damage” refers to the oxidative damage to skin from the same region of the body of a subject of similar race and sex as the skin being diagnosed, albeit not subjected to the effector(s) of oxidative damage.

In one example, the baseline level is taken as the level of oxidative damage determined according to any embodiment described herein in a sample of stratum corneum of a healthy control subject. For example, the healthy control subject may be a subject that has not been exposed to a damaging level of ultraviolet radiation or ionising radiation or a chemotherapeutic agent. In this example, the healthy control may be an age matched control to the subject being diagnosed, but that has not been treated with ionizing radiation, or a chemotherapeutic agent. In another example, the healthy control subject may be a subject of an age that is insufficient to have had aging-related damage to the skin e.g., less than about 25 years of age or less than about 20 years of age or less than about 15 years of age.

In another example, the baseline level is taken as the level of oxidative damage according to any embodiment described herein in a sample of stratum corneum of the subject being diagnosed, albeit where visible symptoms of skin damage are absent, as described supra.

It will be understood that any numerical value greater than 0 for this comparison will be indicative of the presence of oxidative damage in the subject. In one example the numerical value above baseline is between about 0 to about 100; or about 100 to about 200; or about 200 to about 300; or about 300 to about 400; or about 400 to about 500; or about 500 to about 600; or about 600 to about 700; or about 700 to about 800; or about 800 to about 900; or about 900 to about 1000. In another example, the numerical value above baseline is between about 0 to about 50; or about 50 to about 100; or about 100 to about 150; or about 150 to about 200; or about 200 to about 250; or about 250 to about 300; or about 300 to about 350; or about 350 to about 400; or about 450 to about 500.

In another example, the numerical value above baseline is between about 0 to about 10; or about 10 to about 20; or about 20 to about 30; or about 30 to about 40; or about 40 to about 50; or about 50 to about 60; or about 60 to about 70; or about 70 to about 80; or about 80 to about 90; or about 90 to about 100.

### 1.4 Sampling Methods

It will be understood that samples may be collected and/or obtained from the subject, or from the subject and from a healthy control subject by any method known in the art using e.g, by physical scraping, solvent wash, or swabbing/wiping methods

Sample(s) containing lipids from the stratum corneum can be taken from a suitable site of stratum corneum and may be located on any skin surface e.g., a region of arm, leg, face, torso, or back. It will be understood that the sample of stratum corneum is readily obtained by e.g., a skilled technician, or a physician such as a dermatologist. The sample may be obtained by a dermatologist in their office when a subject suffering from symptoms of skin damage is being assessed by the dermatologist.

Samples of stratum corneum may be obtained by scraping the site of stratum corneum with a rigid instrument. It will be recognized that any number of rigid instruments capable of non-invasively removing only the surface layer (i.e., stratum corneum) of the skin can be used, e.g., a plastic scraper or a stick with a cotton bud. For example, the skin is scraped upto 10 times with a plastic scraper or cotton bud or other instrument, cellular material and sebum are then recovered from the plastic scraper, or other instrument using a suitable organic solvent or by any method known in the art. Alternatively, instead of scraping the skin, the skin's epidermal layer may be removed by using an adhesive tape, for example, Duct tape (333 Duct tape, Nashua tape products) or Scotch® tape (3M Scotch 810, St. Paul, Minn.). However, one embodiment of the method is to use D-SQUAME® (CuDerm, Dallas, Tex.) to strip the skin cell layer. For example, the skin is stripped with the tape up to about 10 times, and the stripped material comprising cells, cellular material and sebum are then recovered from the tape. For example, stripped material is recovered using a suitable organic solvent by any method known in the art.

A person skilled in the art will understand what suitable organic solvents may be used for obtaining samples and includes for example, solvents such as acetone, or alcohol-based solvents such as ethanol, or isopropanol, and may also comprise a non-catalytic antioxidant e.g., urate, ascorbate, α-tocopherol, or bilirubin.

In another example, a towelette soaked in organic solvent is used, e.g., a cloth or paper wipe, such as cheese cloth, tissue paper, or filter paper, or any wipe known in the art that is suitable for wiping skin. In this example, the towelette is soaked in a suitable organic solvent as described supra. The towelette may contain other agents, such as mild detergents that are used for cleansing skin. In one embodiment, said wipe does contain agents that oxidize the material present in the sample. The towelette is then used to wipe the stratum corneum. This wiping process may be repeated up to 10 times. The towelette is then soaked with further organic solvent sufficient to cover the wipe in a suitable container. The organic solvent comprising cells, cellular material and sebum is decanted after about 1 to about 10 minutes.

In another example, the stratum corneum is washed directly with a suitable organic solvent as described supra. A syringe barrel is used to flush skin directly and the run-off is collected, and pooled. For example, about 1 ml of organic solvent in a 2 ml syringe barrel may be used and flushed up to 10 times on the surface of a site of stratum corneum directly into a 50 ml conical polypropylene tube.

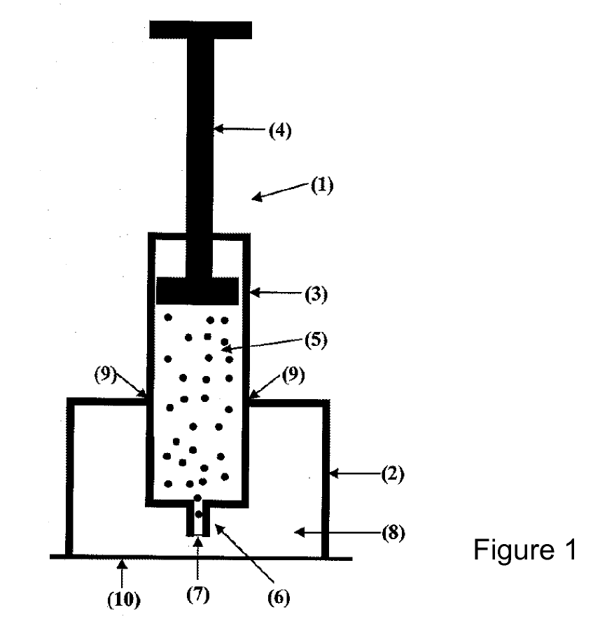

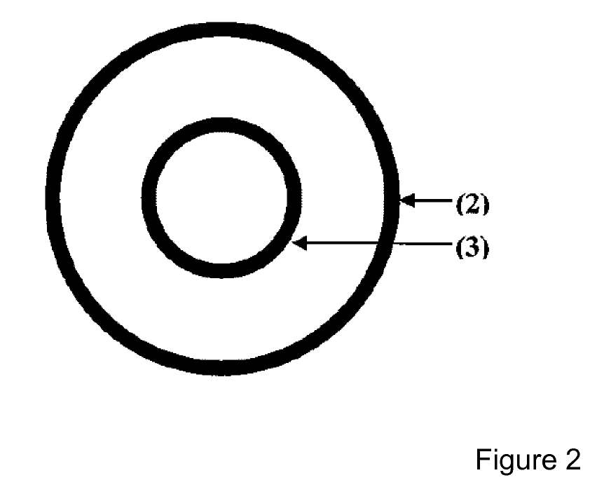

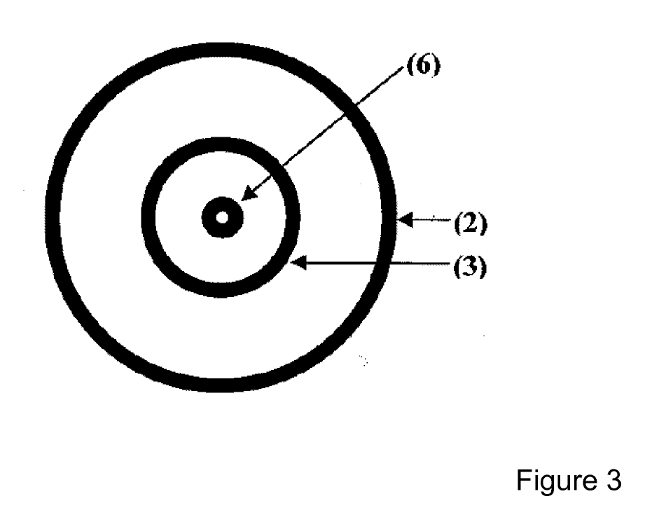

In another example, the sample of stratum corneum can be collected by organic solvent wash of the stratum corneum using a medical device as shown in FIGS. 1 to 4. The medical device can be used as an apparatus for diagnosing a skin condition.

Referring to FIG. 1, the medical device can comprise a syringe member (1) and a base member (2). The syringe member comprises a cylinder (3), a piston rod (4) provided within the cylinder said piston rod (4) guided by the wall of the cylinder provides for a clearance of solution (5), and an opening (6) at the proximal end that is covered by a removable membranous member (7). Prior to sample collection, the cylinder comprises the solution (5) suitable for collecting stratum corneum samples. Said solution (5) can comprise a solvent, such as an organic solvent such as acetone, or alcohol-based such as ethanol, or isopropanol and comprising a non-catalytic antioxidant e.g., urate, ascorbate, α-tocopherol, or bilirubin. The cylinder (3) can be capable of holding a volume of at about 1.5-2.5 ml. In one embodiment, the cylinder holds more than about 1.5 ml. In another embodiment, the cylinder holds more than about 2 ml. The base member (2) can comprise a reservoir (8) adapted for fluid communication with a region of skin. The reservoir further houses the proximal end of the cylinder portion (6) such that the distal end of the reservoir and the wall of the cylinder are communicatively coupled (9). The reservoir (8) is capable of holding the volume of solution (5). It can be useful for the reservoir (8) to hold a volume of about 2.5 to 5 ml. In one embodiment, the reservoir (8) holds a volume of about 3 to 4 ml. In another embodiment, the reservoir (8) holds a volume of about 4 ml. The proximal end of the reservoir can be open and capable of contacting the skin of a subject such that it provides a sealed contact with the skin when in use. The proximal end also includes a removable lid assembly (10) wherein the lid is closed prior to use, and opened just before use. It can be useful for the lid assembly to be easily removed just before use. Any lid assembly that is easily removed just before use can be contemplated, and a useful form can comprise a protective membrane that is peeled off prior to use.

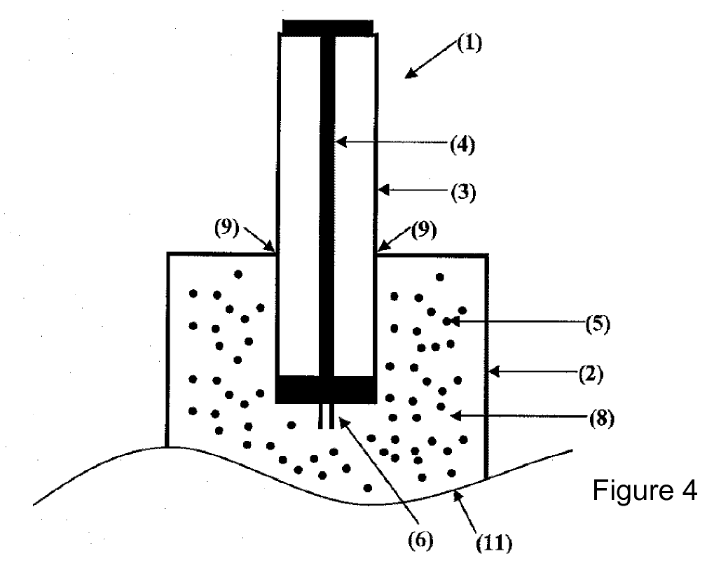

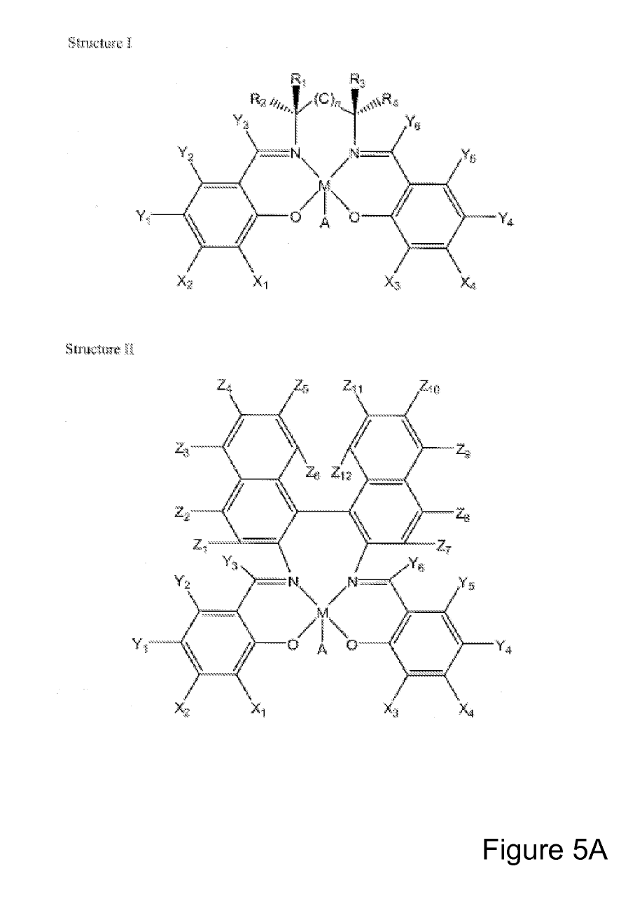

Referring to FIG. 4 showing a schematic diagram of the device in use, the protective membrane or lid assembly of the base is removed and the open-end of the base member (2), or alternatively a skin contacting lip, can be placed on the surface of a suitable site of stratum corneum such that the open-end of the reservoir (8) makes contact with the skin surface of the subject (11) and makes a seal that prevents liquid leaking from the reservoir by pressure exerted by the dermatologist taking the skin sample. It is useful if there is minimal leakage of solution (5) from the reservoir, such as between 0.1 to 0.5 ml of organic solvent solution whilst in operation. Once the seal is made, the dermatologist then exerts pressure on the piston rod (4) clearing the solution (5) from the cylinder (3) of the syringe portion (1) and this removes the membrane seal (7) at the opening (6) of the cylinder allowing the solution (5) to enter the reservoir (8) of the base member (2) and make contact with the suitable site of stratum corneum of the skin of the subject (11). The device is held in place for about 1 sec to about 5 min. In one example, the device is held in place for about 1 to 120 sec. In another example, the device is held in place for about 5 to 30 sec. In yet another example, the device is held in place for about 10 sec. The solution is then removed from the reservoir (8) by exerting a pulling force on the piston rod (4) such that the solution is enters the cylinder (3) and the device is removed from the subject. The solution may then be transferred to a suitable vial.

In another embodiment, an apparatus for diagnosing a skin condition can be used. The apparatus can include a skin contacting member configured to remove a sample from a region of skin. The skin contacting member can be configured to non-invasively remove only the surface layer (i.e., stratum corneum) of the skin, e.g., a plastic scraper, cotton swab, adhesive tape and solvent carrying towelette. The apparatus can further comprise a channel for housing a solvent suitable solvent, such as acetone, or alcohol such as ethanol, or isopropanol and comprising a non-catalytic antioxidant e.g., urate, ascorbate, α-tocopherol, or bilirubin. A single channel can be used for introducing the solvent and withdrawing the solvent after contact with the region of skin. The apparatus can also be configured with more than one channel. One example is the apparatus housing one channel to introduce the solvent and another channel for removing the solvent.

Samples of stratum corneum may then be subjected to further analysis for the presence of non-oxidated products and/or oxidated products, such as oxidized or peroxidized lipids, for example as described supra and exemplified herein. For example, the final sample in organic solvent solution is centrifuged to separate insoluble material or alternatively, they can be filtered through hydrophobic polypropylene filters (ReZist) with a 0.45 μm pore diameter (or other filters provided that their affinity for solvent-soluble material is checked carefully) and the solvent sample is evaporated in a Speed-Vac vacuum evaporator (Savant, Holbrook, N.Y.).

In another embodiment, the apparatus can be configured with a collection chamber. The collection chamber can be used to store the sample removed from the region of skin. Alternatively, the collection chamber can contain a solvent, such as an organic solvent for separation of insoluble material or a reagent that binds to one or more oxidized substances. The apparatus can further include an absorptive medium for holding the solvent after extraction or separation of the materials from the sample. Additional embodiments may desire the collection chamber to be adapted to inject the sample into an analysis apparatus, such as a centrifuge, chromatography column, mass spectrometer or filter.

### 2. Therapeutic Regimes

### 2.1 SOD/Catalase Mimetics

A synthetic SOD/catalase mimetic recommended according to any example hereof may be a salen-transition metal complex as described in any one of U.S. Pat. Nos. 5,403,834; 5,827,880; 5,696,109; 5,834,509; 6,589,948; 7,122,537; and 6,403,788; and US Publication No. 2007/0123503; and International Publication Nos: WO 94/13300; WO 96/40149; WO 96/40148; WO 2005/000854; and WO 2008/033444.

For example, the SOD/catalase mimetic possesses detectable catalase activity in vitro e.g., as compared relative to SOD/catalase mimetic compound C7 (EUK-8; described infra). In one example, the SOD/catalase mimetic is selected based on having a detectable catalase activity between about 20% to about 100% relative to C7 (EUK-8); or about 100% to about 200% relative to C7 (EUK-8); or about 200% to about 300% relative to C7 (EUK-8); or about 300% to about 400% relative to C7 (EUK-8); or about 400% to about 500% relative to C7 (EUK-8).

In another example, the SOD/catalase mimetic is selected based on having a detectable catalase activity between about 20% to about 50% relative to C7 (EUK-8); or about 50% to about 100% relative to C7 (EUK-8); or about 100% to about 150% relative to C7 (EUK-8); or about 150% to about 200% relative to C7 (EUK-8); or about 250% to about 300% relative to C7 (EUK-8); or about 350% to about 400% relative to C7 (EUK-8); or about 450% to about 500% relative to C7 (EUK-8).

In another example, the SOD/catalase mimetic is selected based on having a detectable catalase activity between about 20% to about 35% relative to C7 (EUK-8); or about 35% to about 50% relative to C7 (EUK-8); or about 50% to about 65% relative to C7 (EUK-8); or about 65% to about 80% relative to C7 (EUK-8); or about 80% to about 95% relative to C7 (EUK-8); or about 95% to about 110% relative to C7 (EUK-8); or about 110% to about 125% relative to C7 (EUK-8); or about 125% to about 140% relative to C7 (EUK-8); or about 140% to about 155% relative to C7 (EUK-8); or about 155% to about 170% relative to C7 (EUK-8); or about 170% to about 185% relative to C7 (EUK-8); or about 185% to about 200% relative to C7 (EUK-8); or about 215% to about 230% relative to C7 (EUK-8); or about 230% to about 245% relative to C7 (EUK-8).

In another example, the SOD/catalase mimetic is selected based on having a detectable catalase activity between about 20% to about 30% relative to C7 (EUK-8); or about 30% to about 40% relative to C7 (EUK-8); or about 40% to about 50% relative to C7 (EUK-8); or about 50% to about 60% relative to C7 (EUK-8); or about 60% to about 70% relative to C7 (EUK-8); or about 70% to about 80% relative to C7 (EUK-8); or about 80% to about 90% relative to C7 (EUK-8); or about 90% to about 100% relative to C7 (EUK-8); or about 100% to about 110% relative to C7 (EUK-8); or about 110% to about 120% relative to C7 (EUK-8); or about 120% to about 130% relative to C7 (EUK-8); or about 130% to about 140% relative to C7 (EUK-8); or about 140% to about 150% relative to C7 (EUK-8); or about 150% to about 160% relative to C7 (EUK-8); or about 160% to about 170% relative to C7 (EUK-8); or about 170% to about 180% relative to C7 (EUK-8); or about 180% to about 190% relative to C7 (EUK-8); or about 190% to about 200% relative to C7 (EUK-8).

Exemplary SOD/catalase mimetics that possess detectable catalase activity in vitro and that fall within the bands of catalase activity relative to C7 (EUK-8) as described supra are shown in Tables 1 to 3.

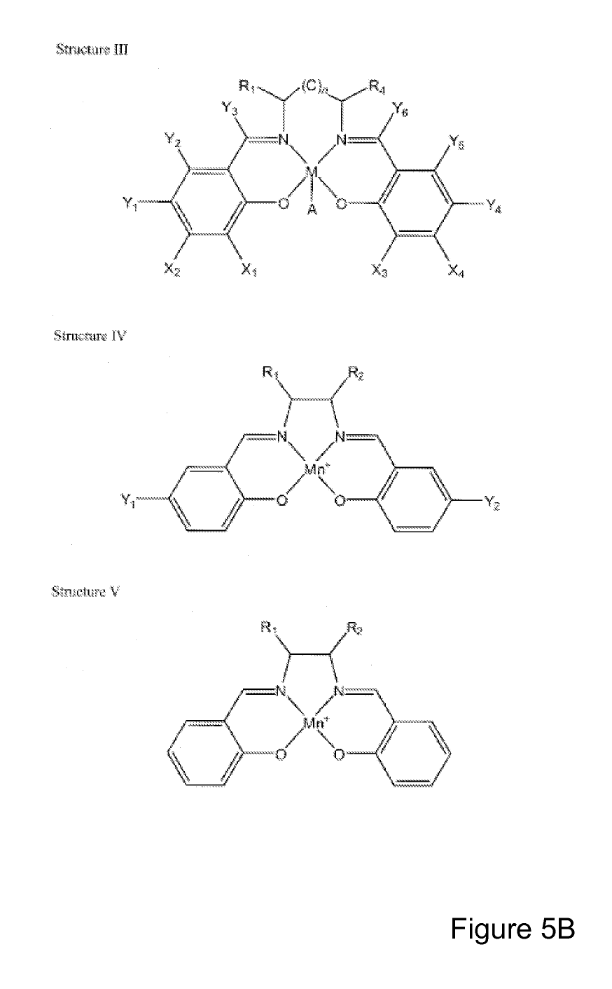

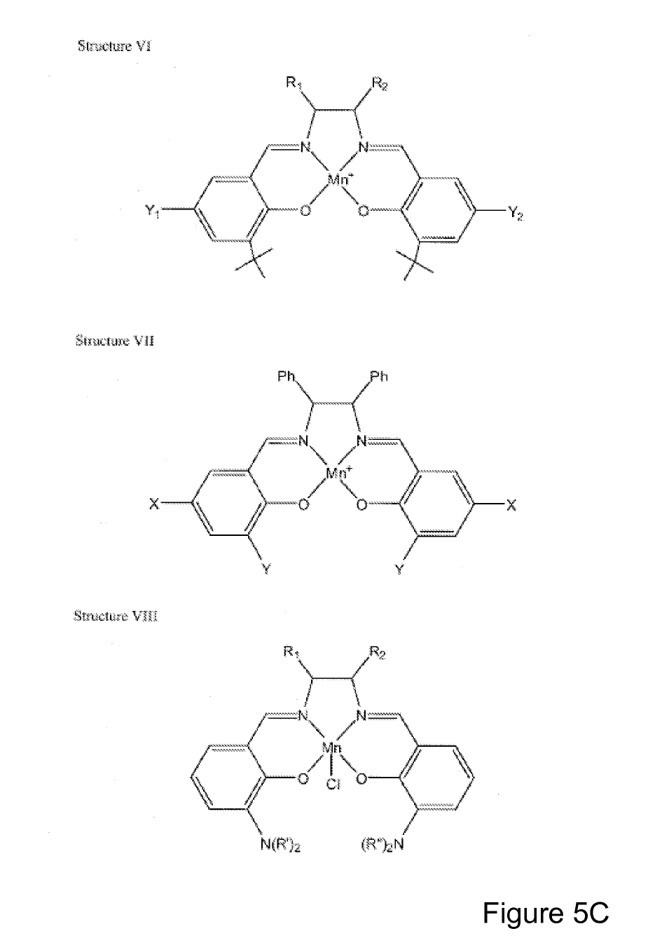

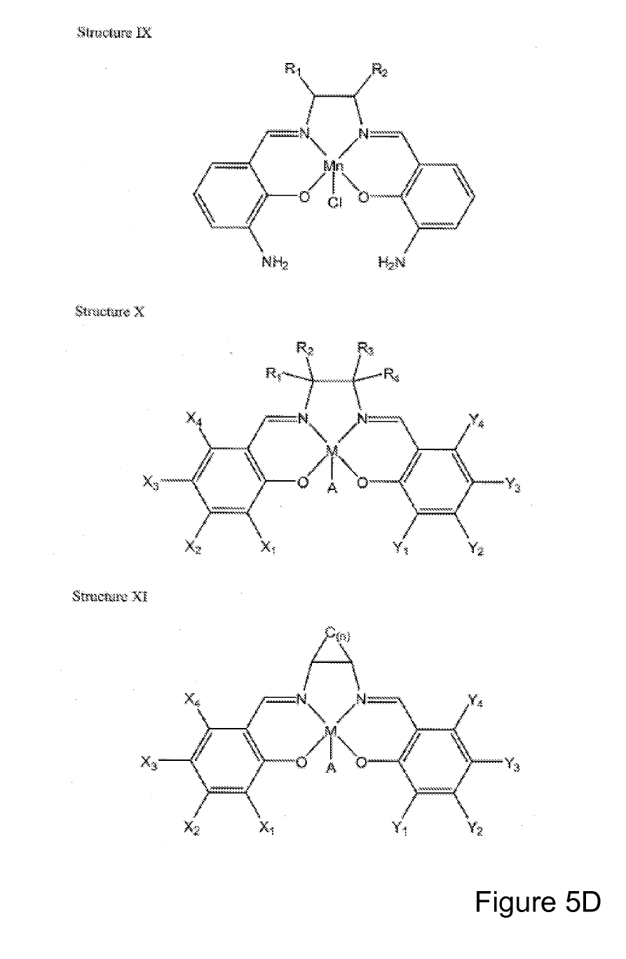

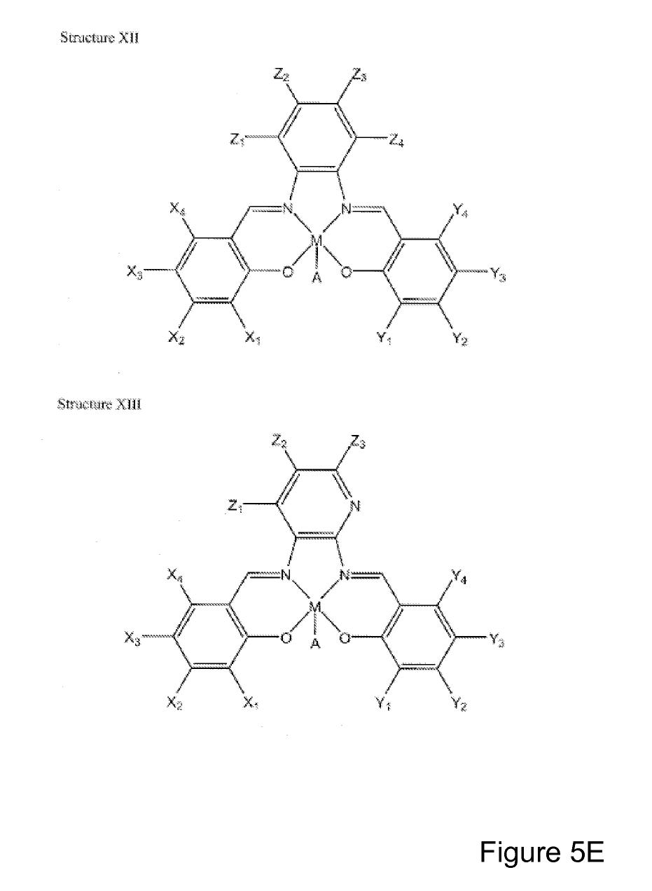

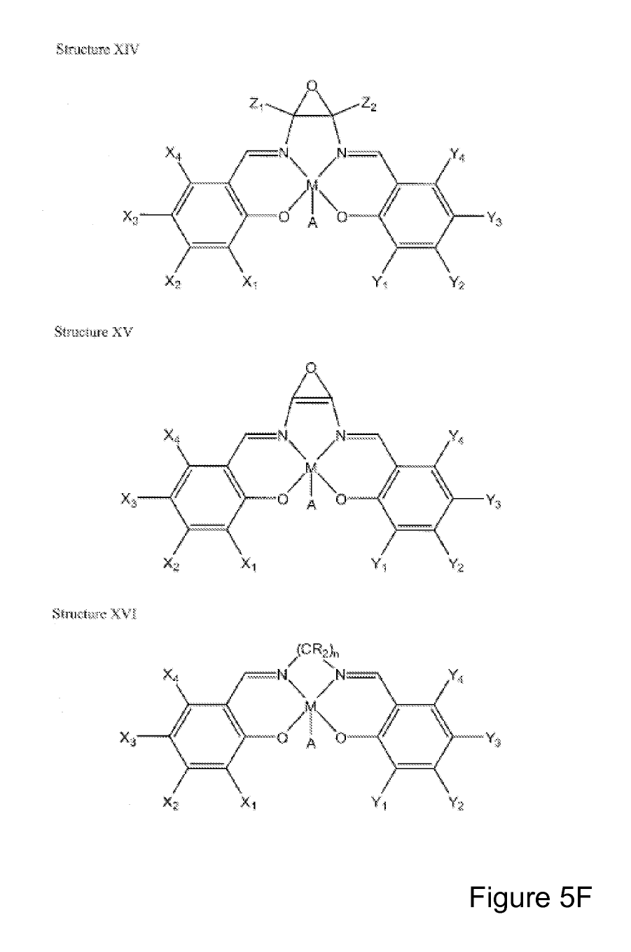

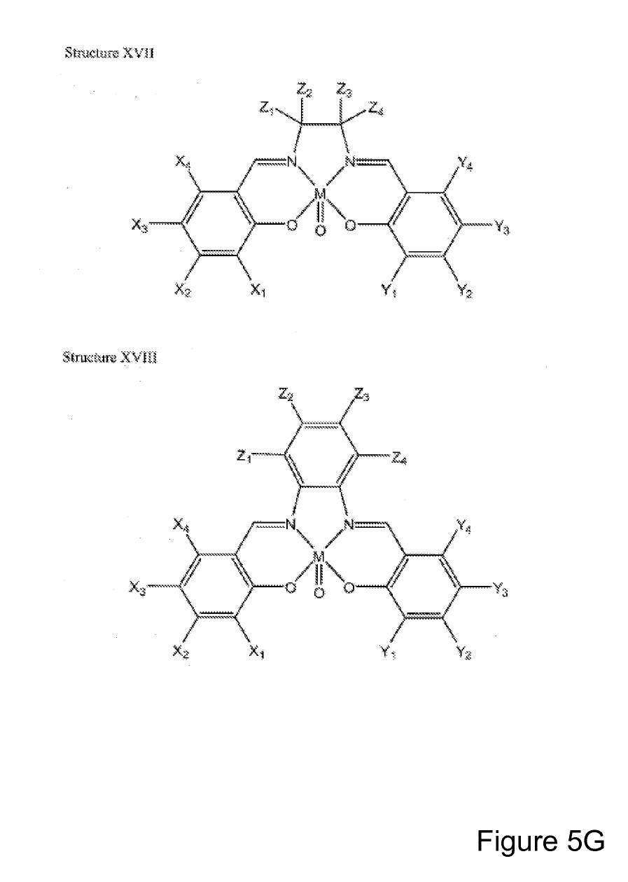

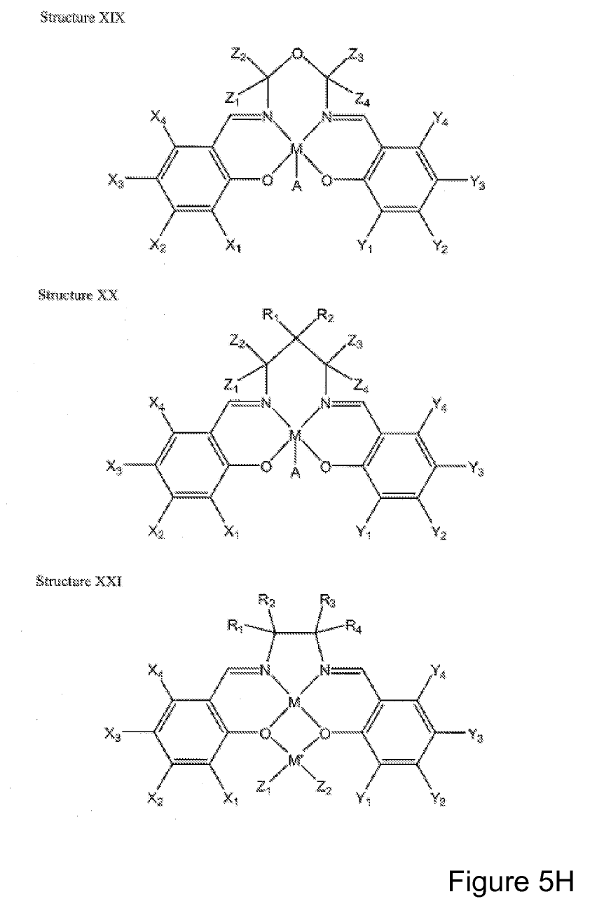

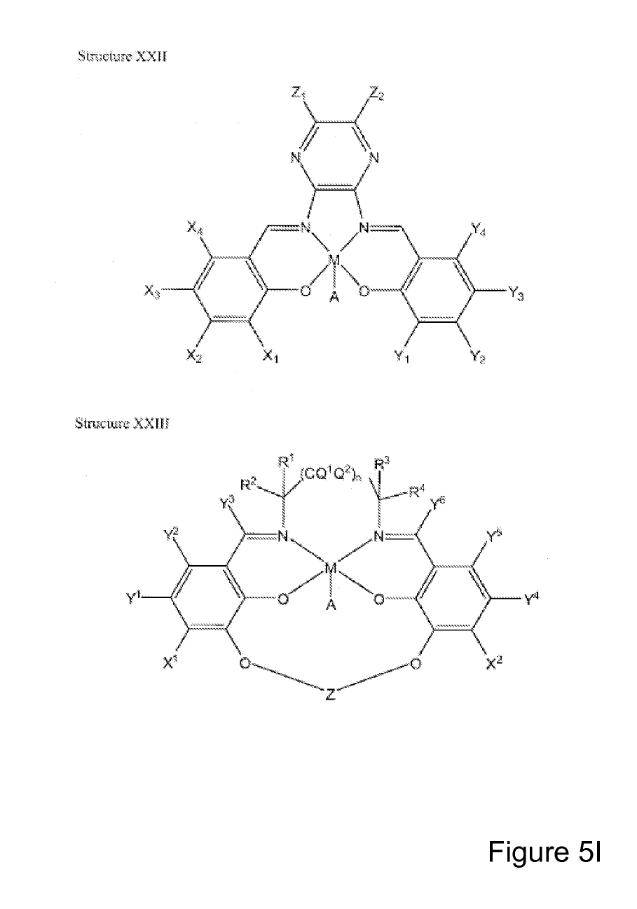

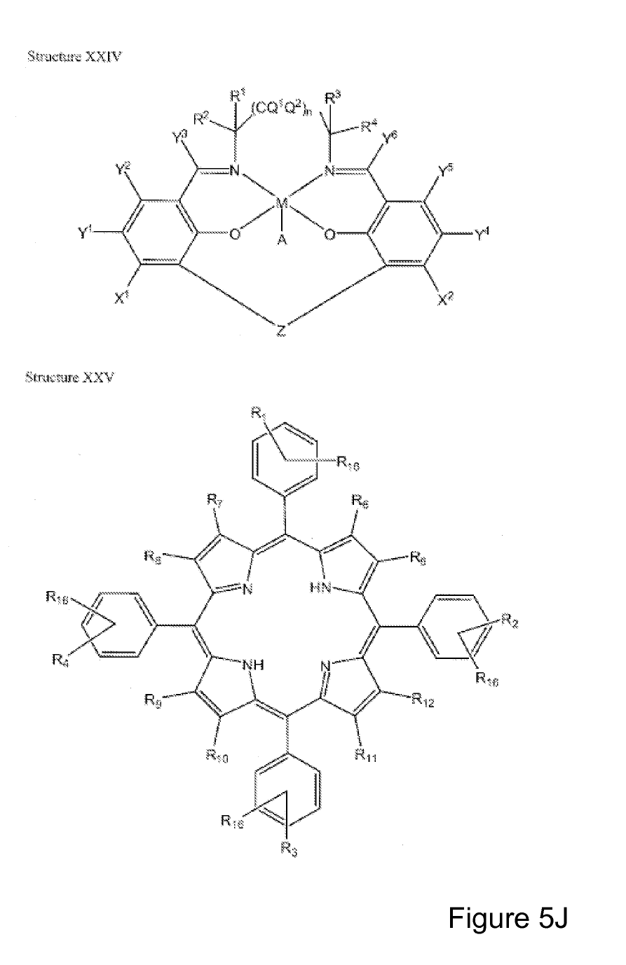

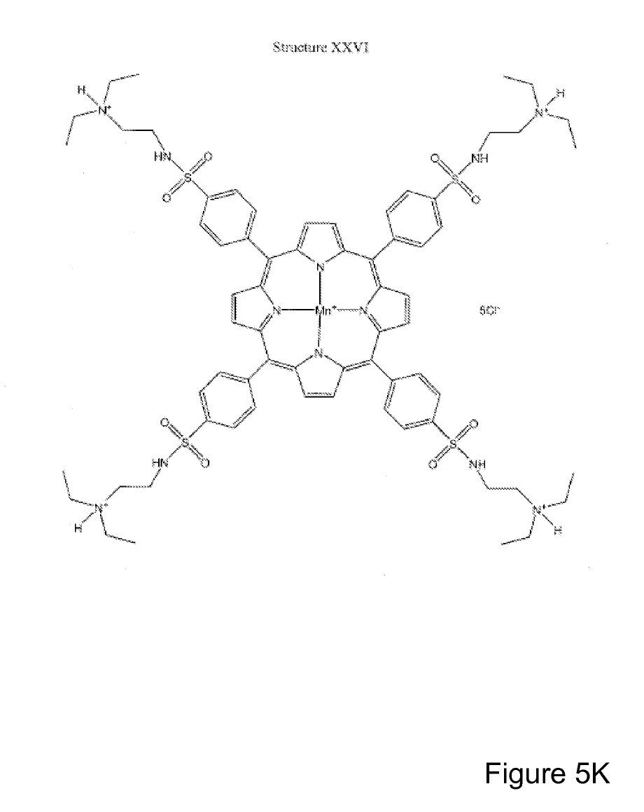

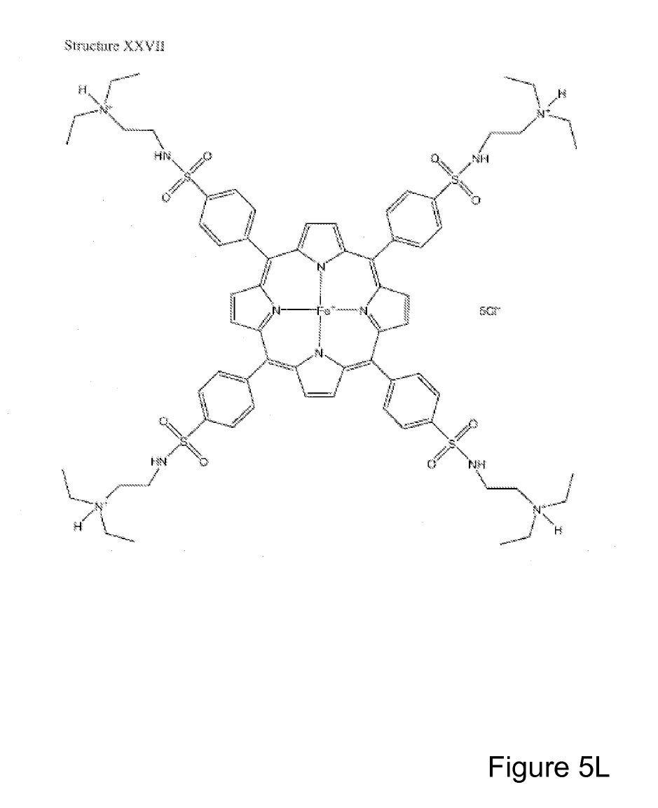

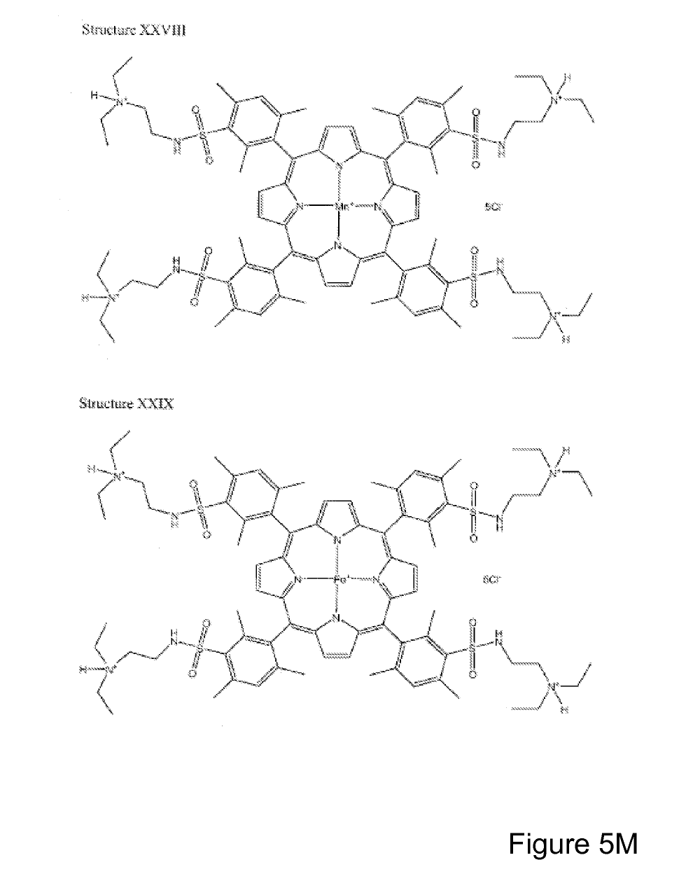

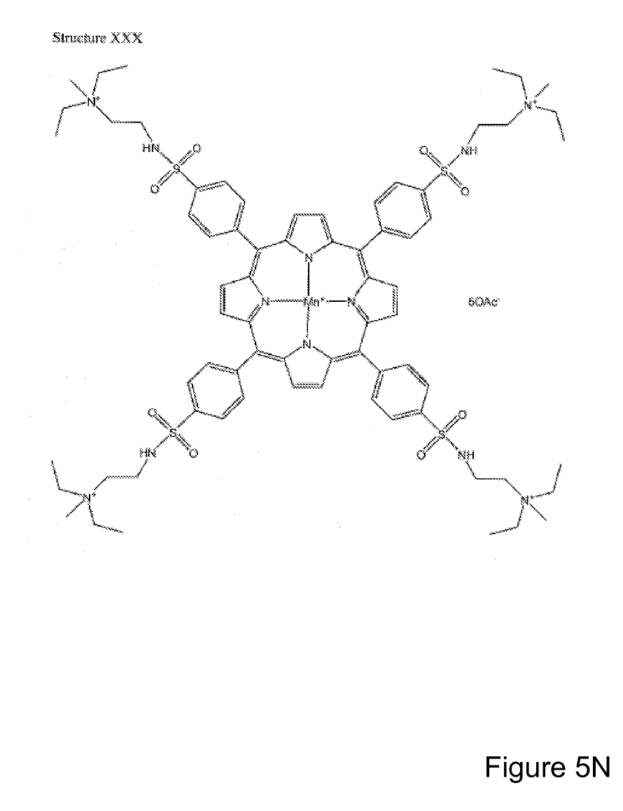

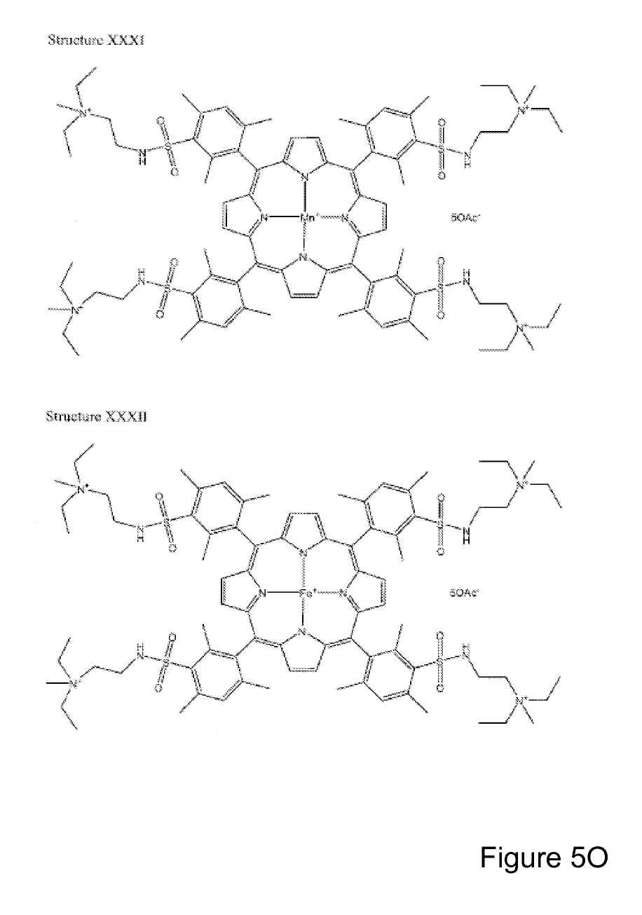

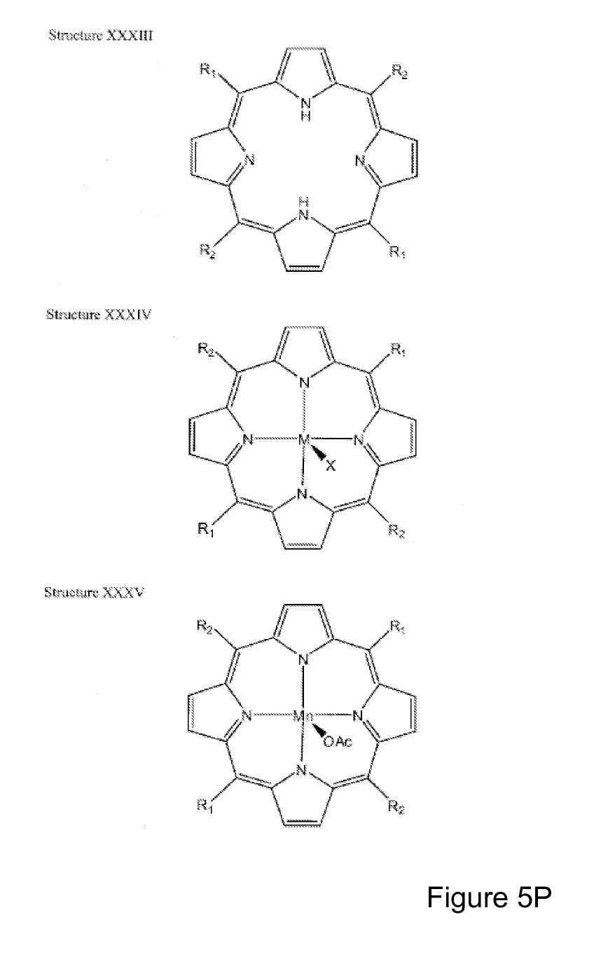

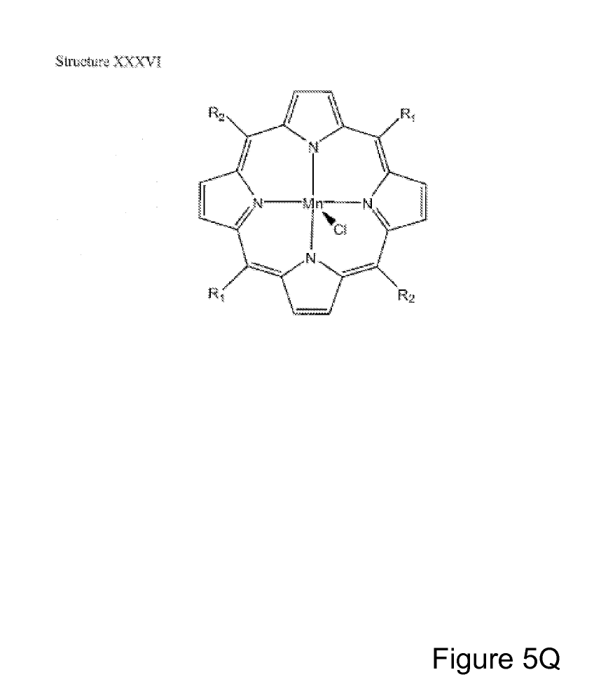

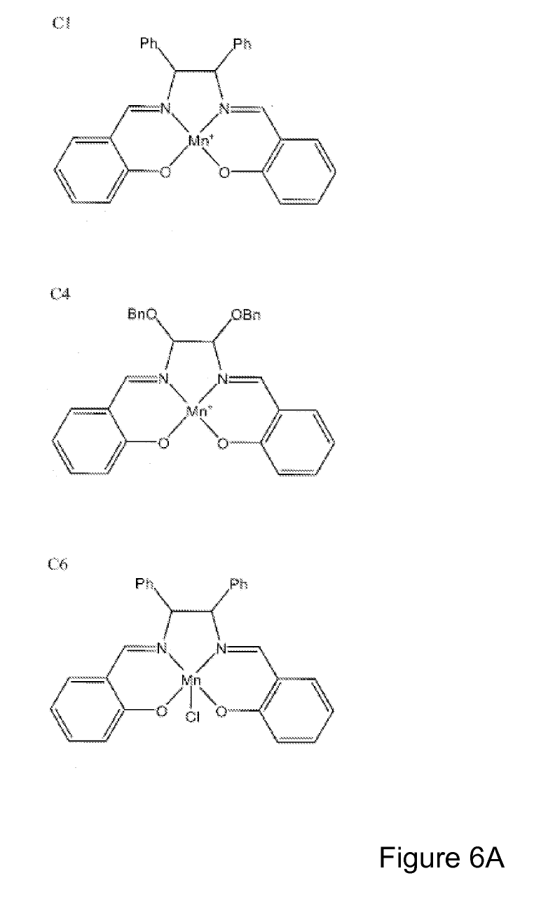

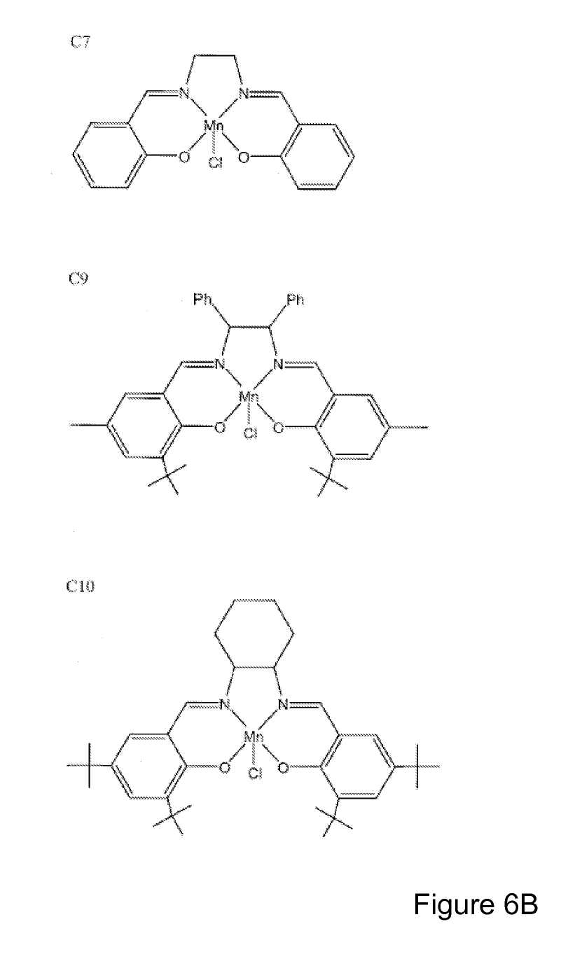

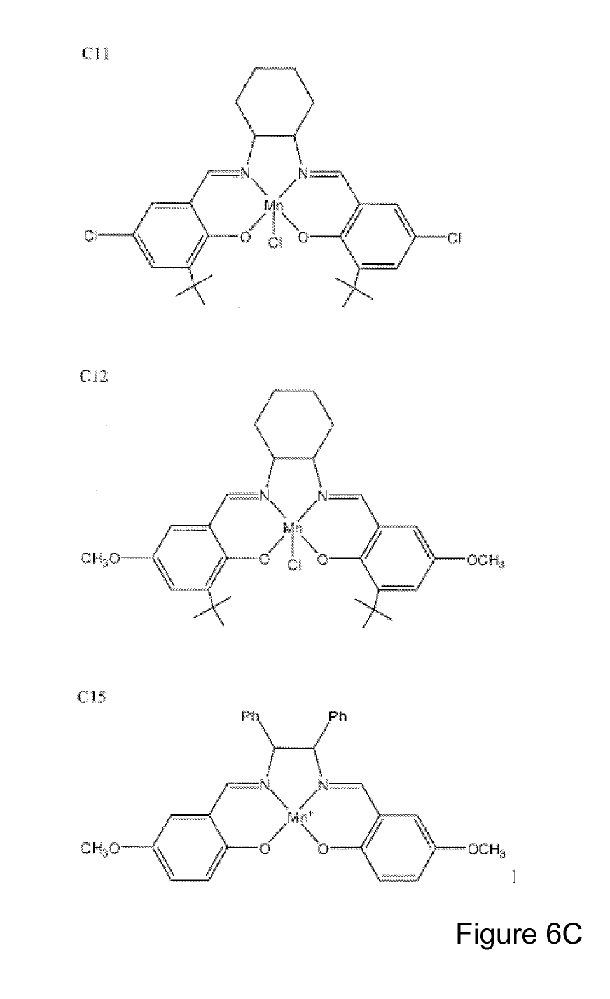

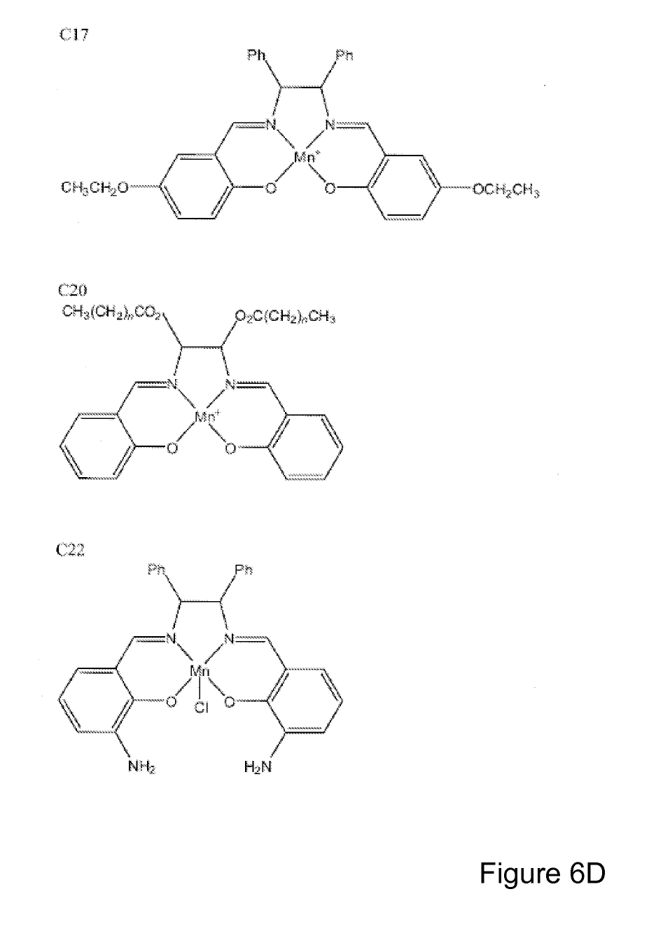

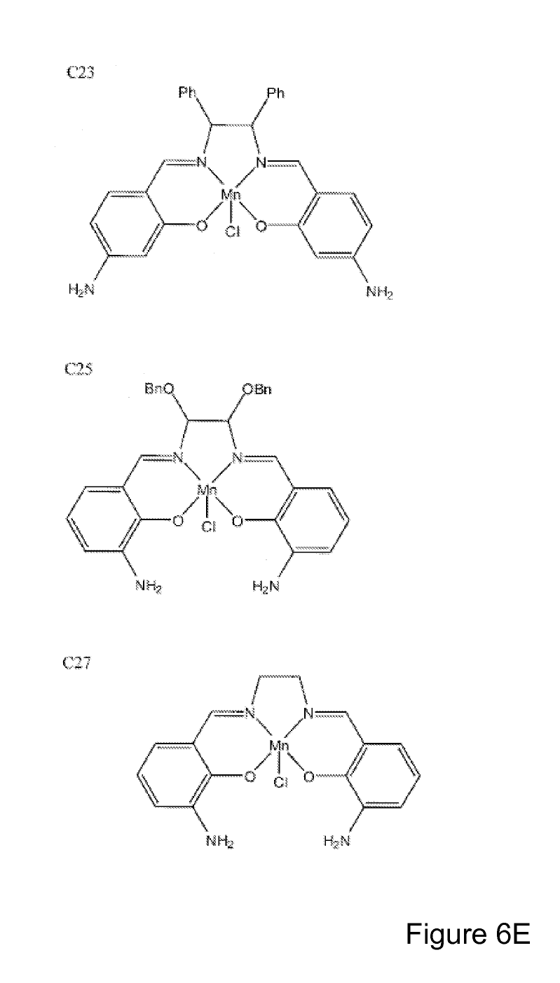

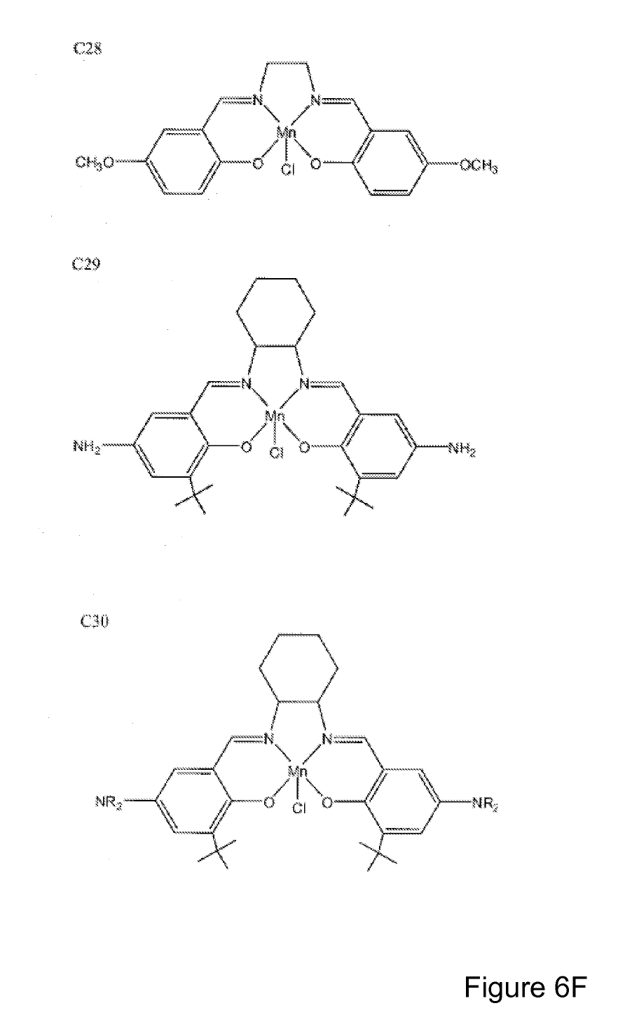

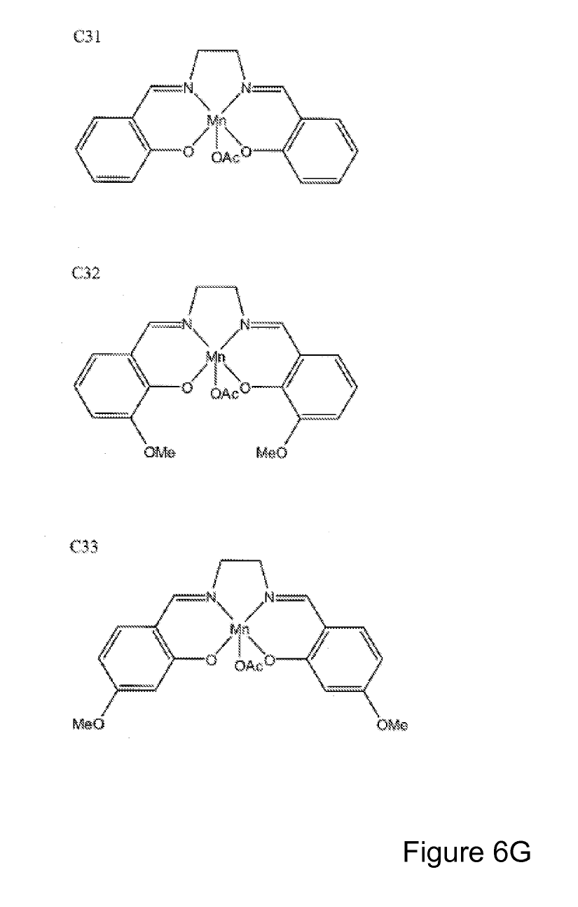

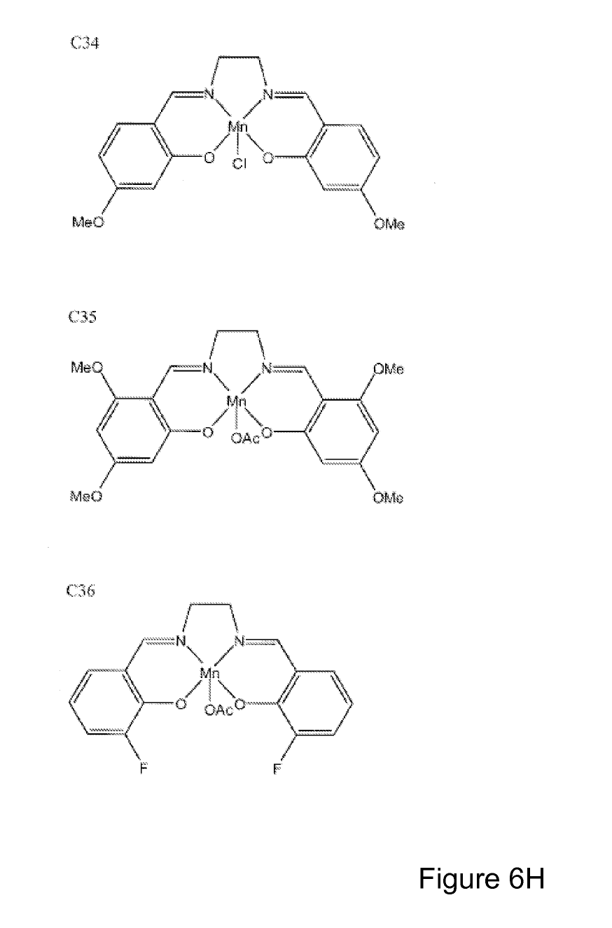

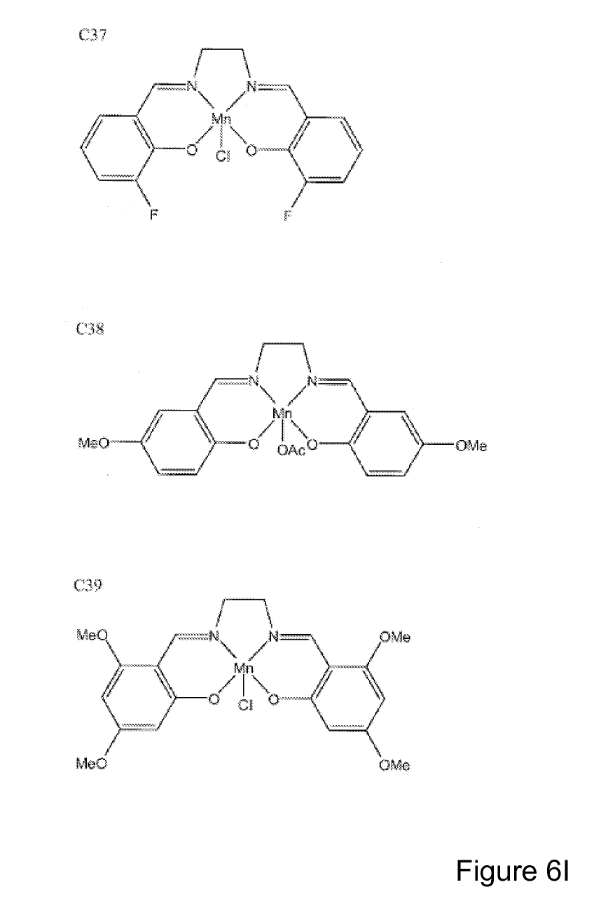

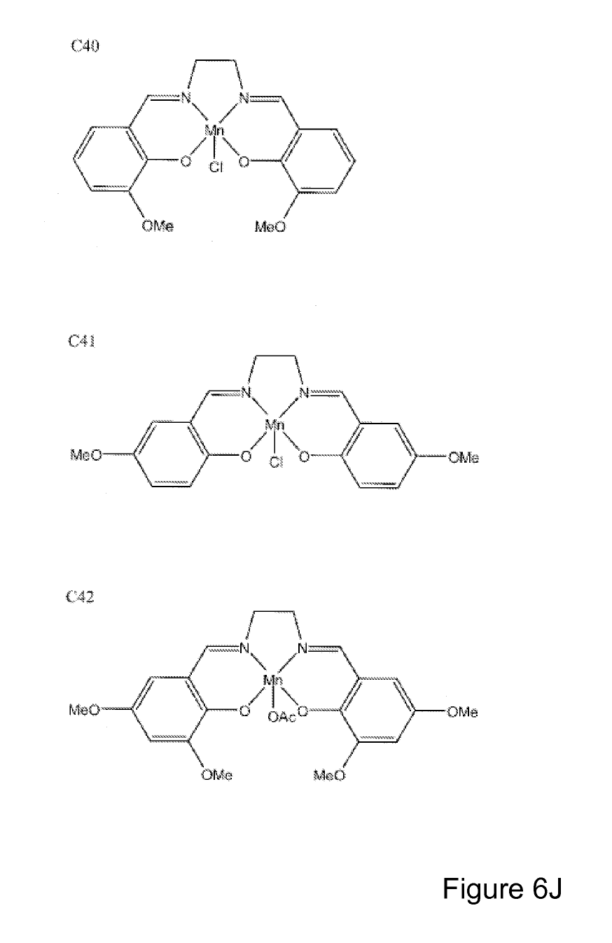

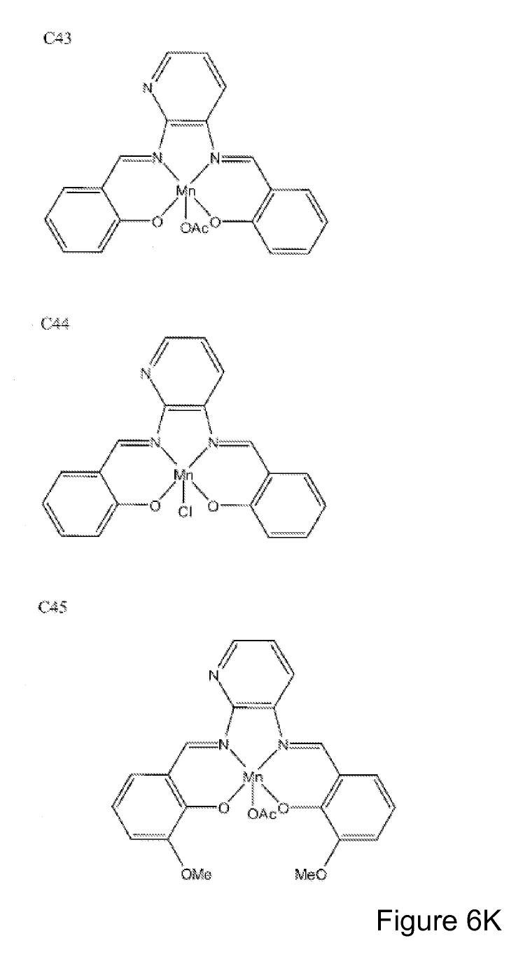

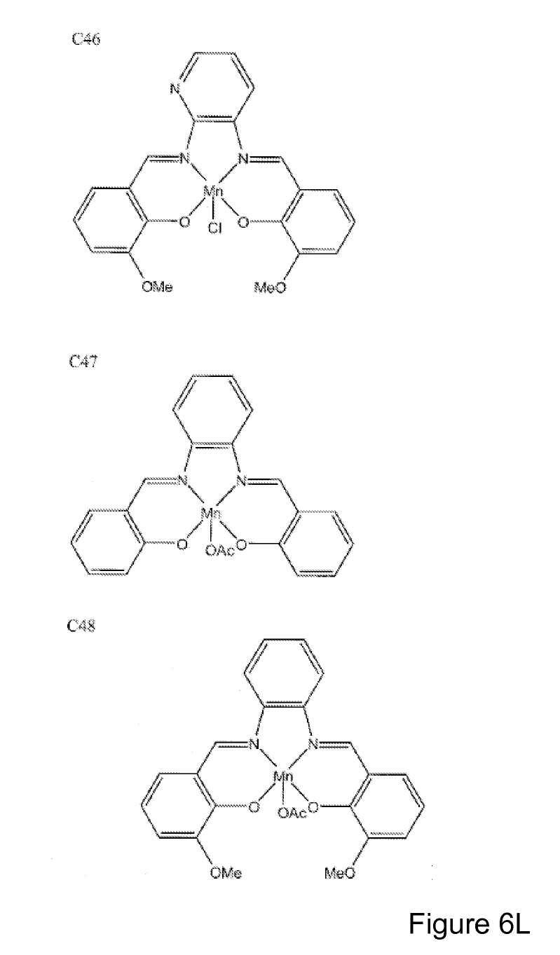

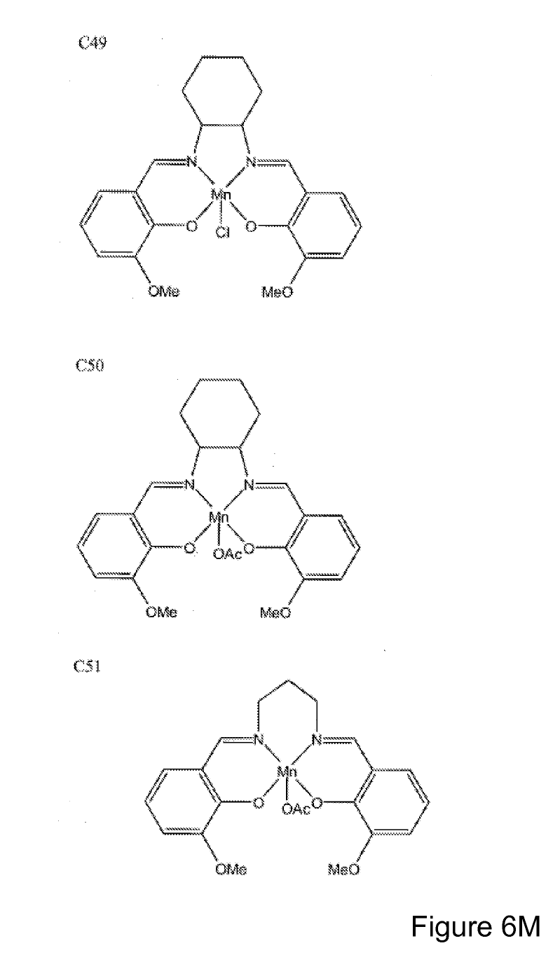

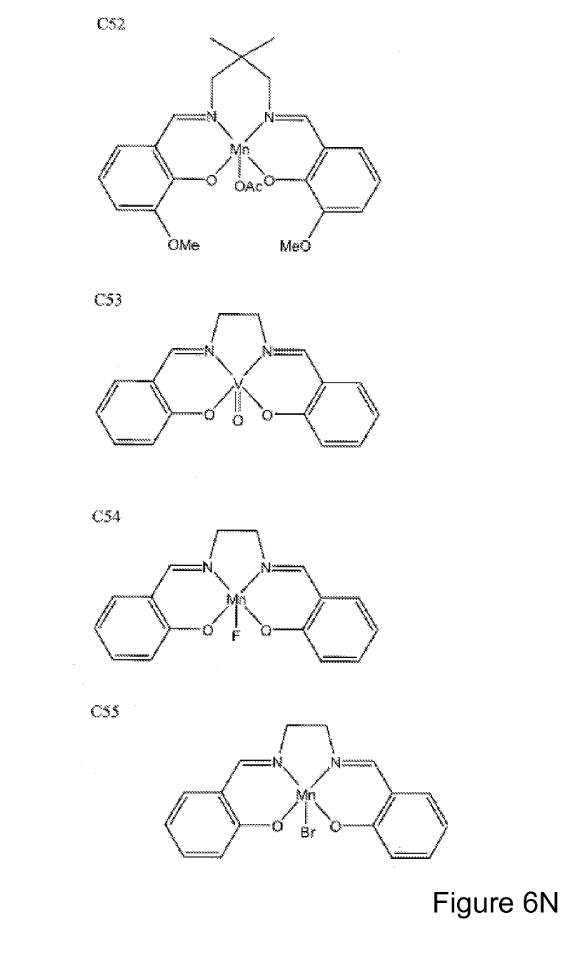

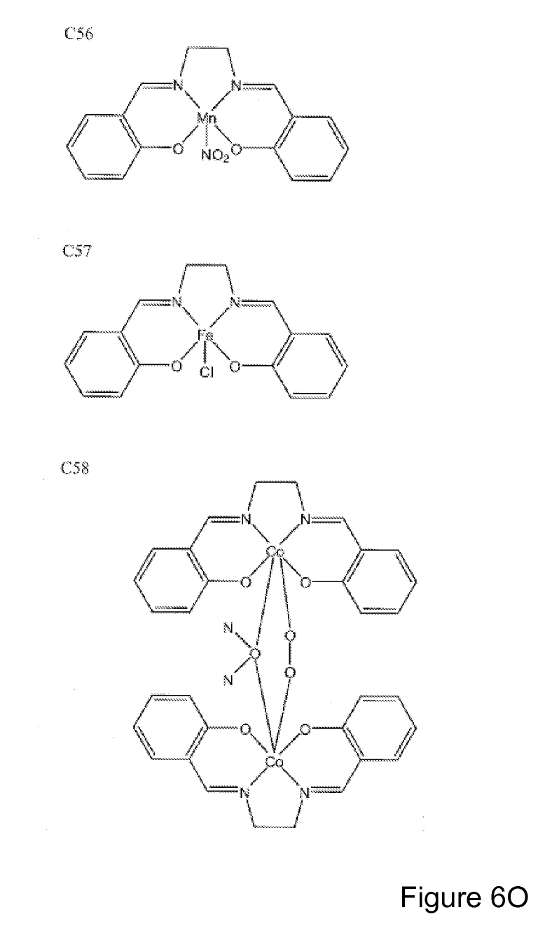

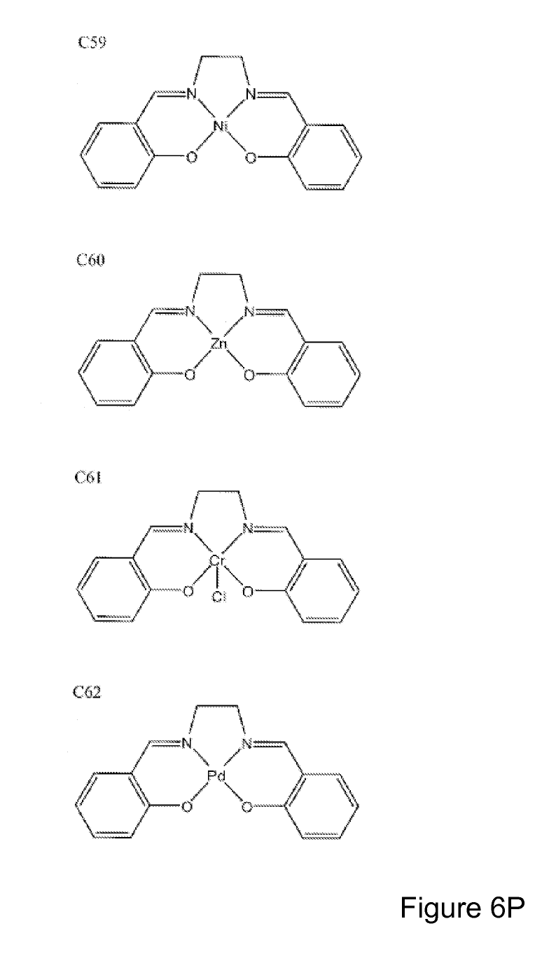

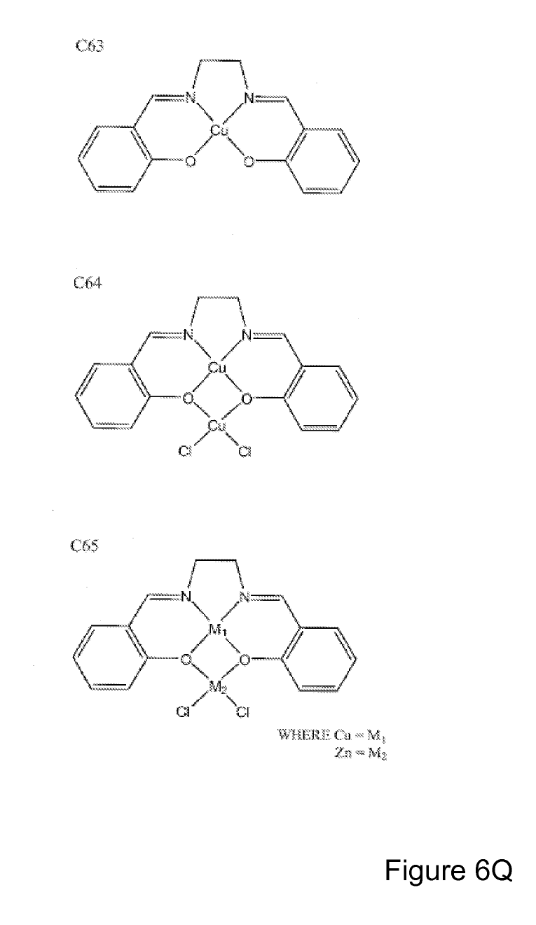

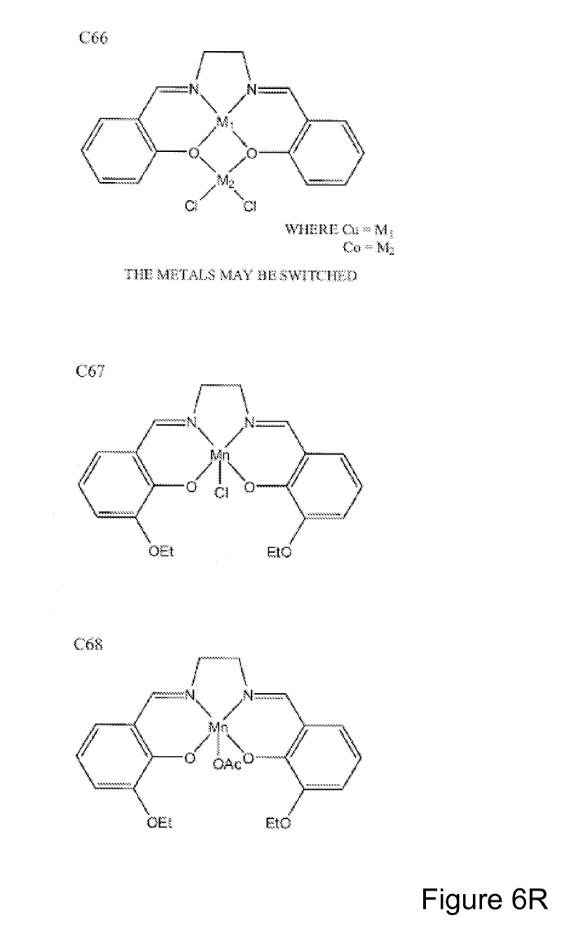

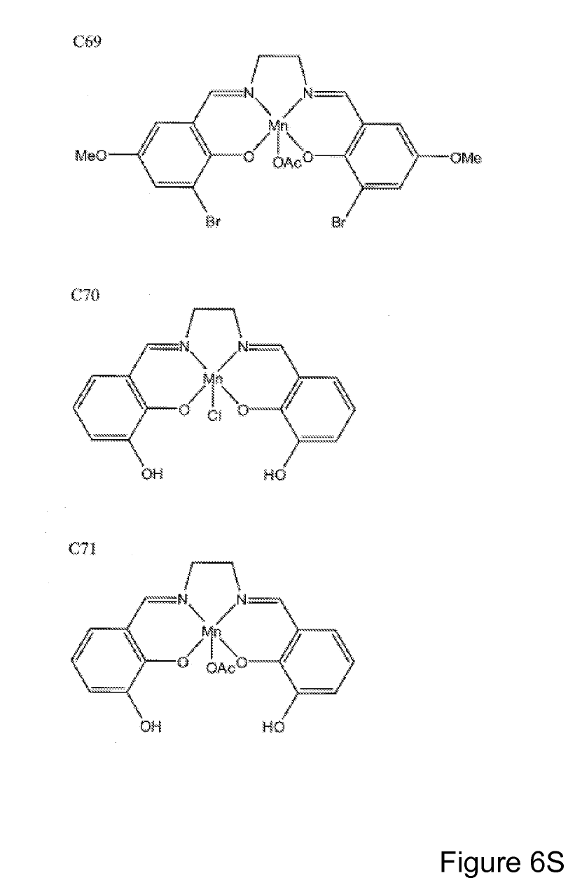

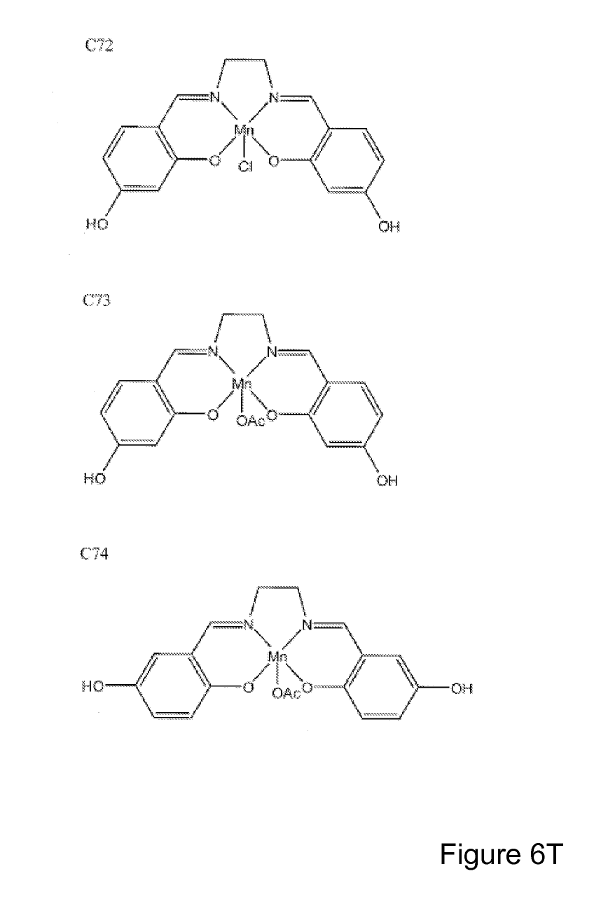

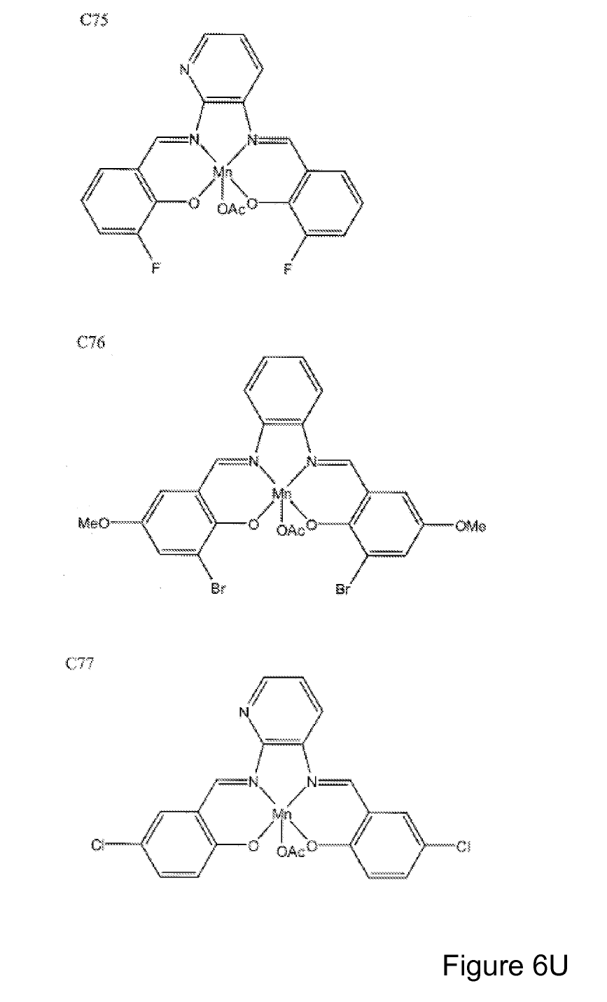

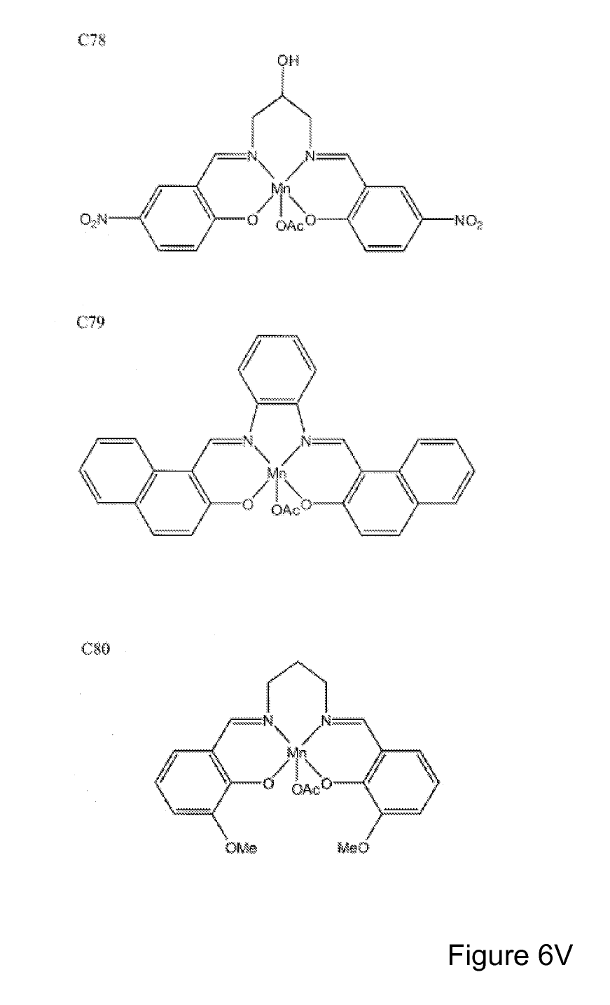

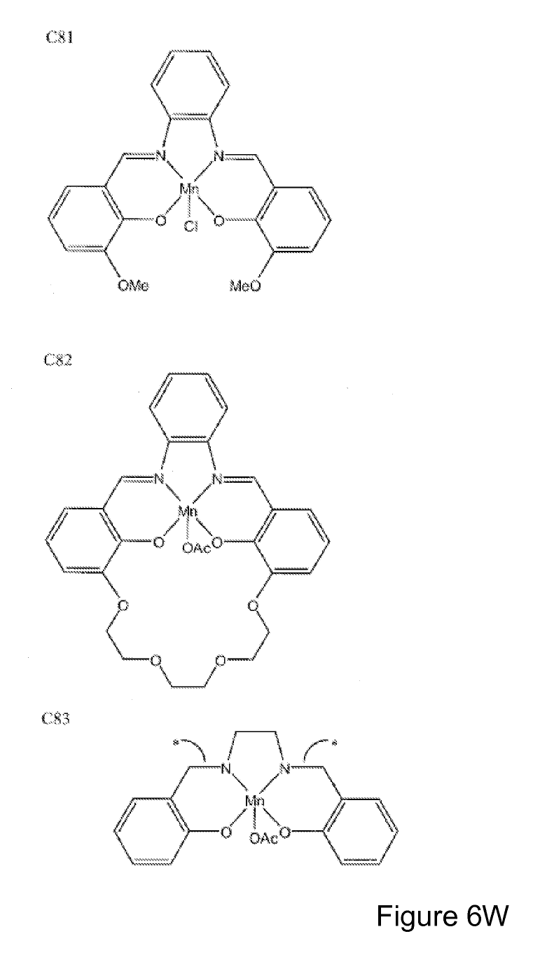

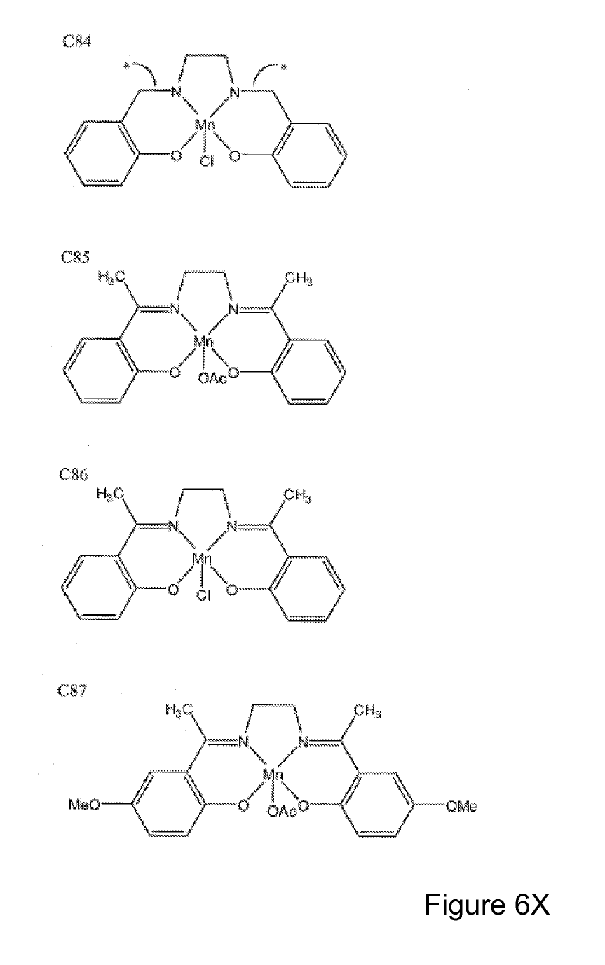

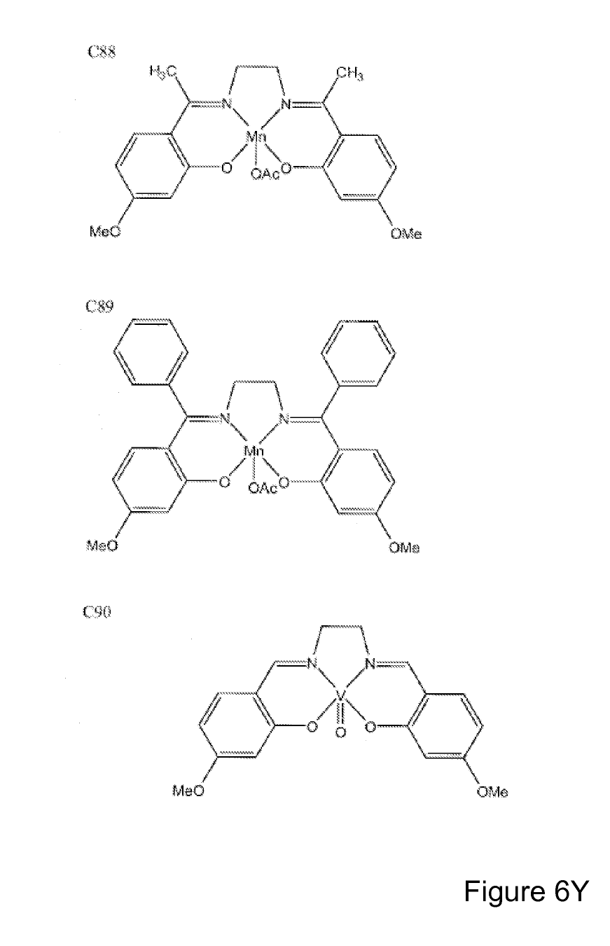

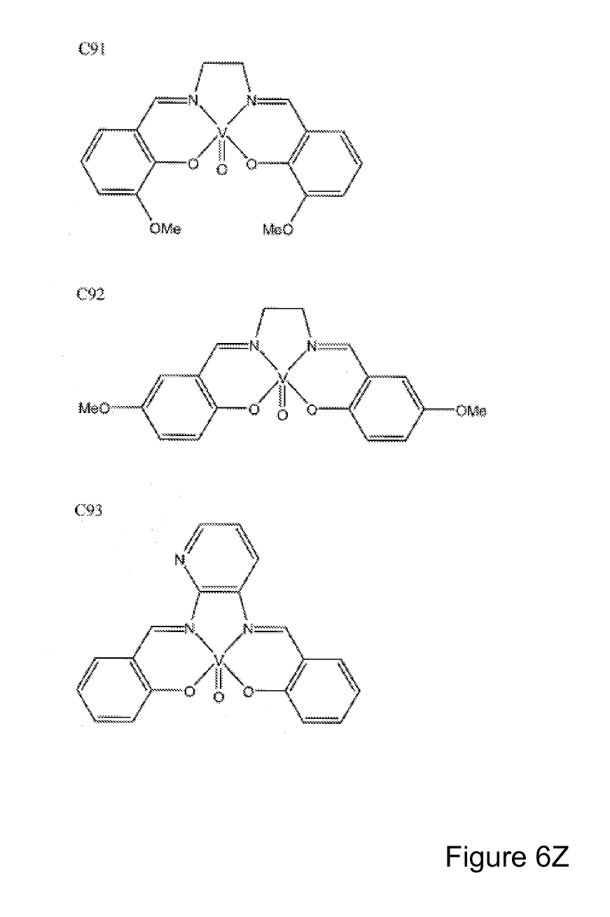

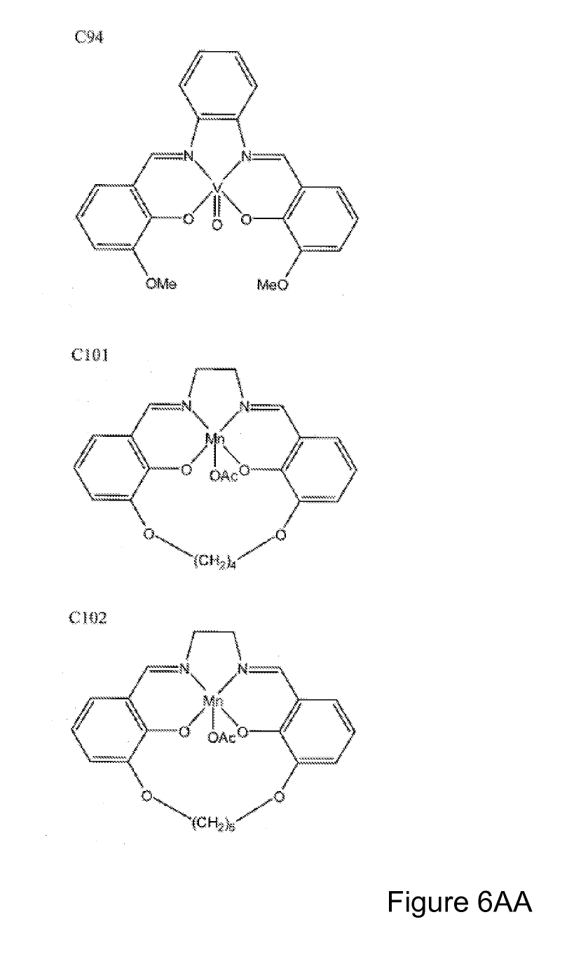

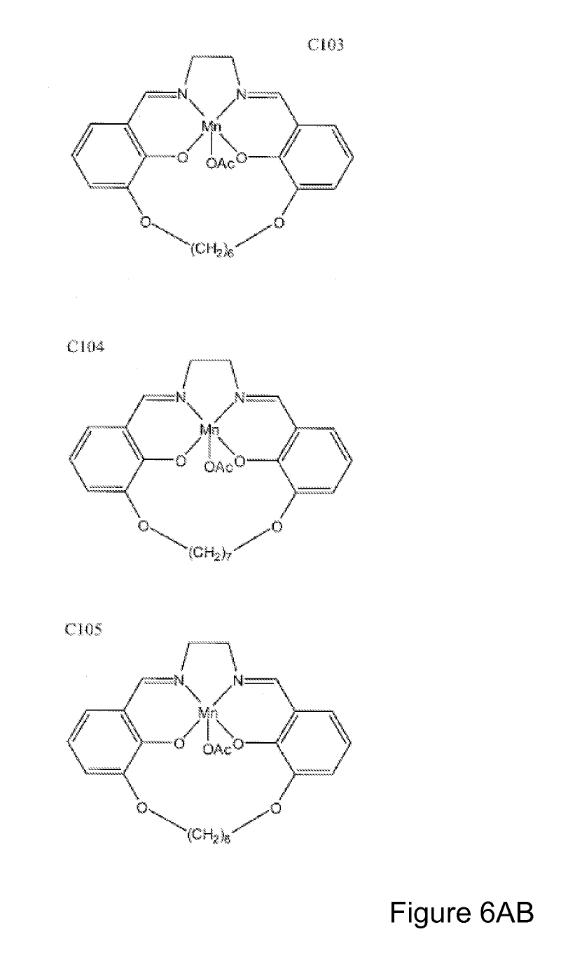

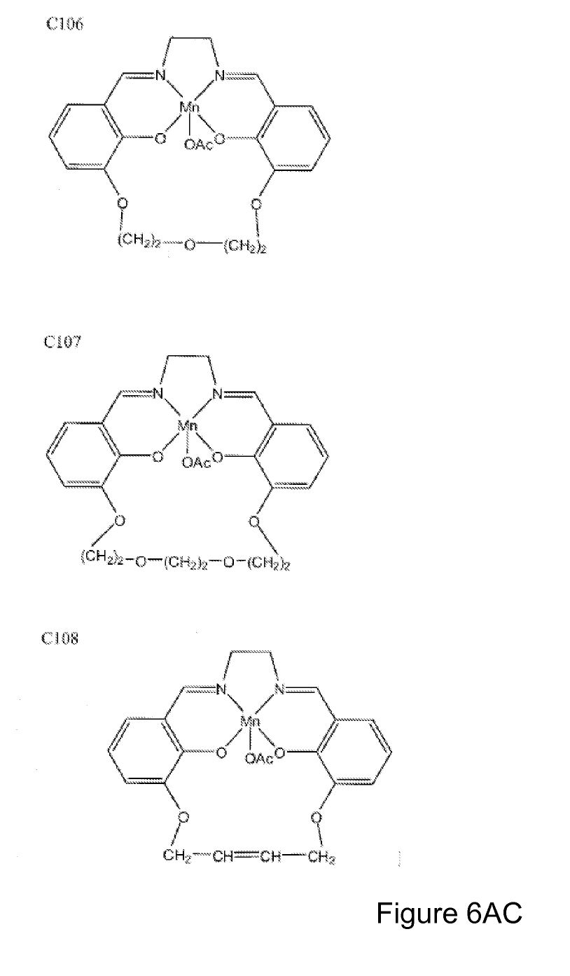

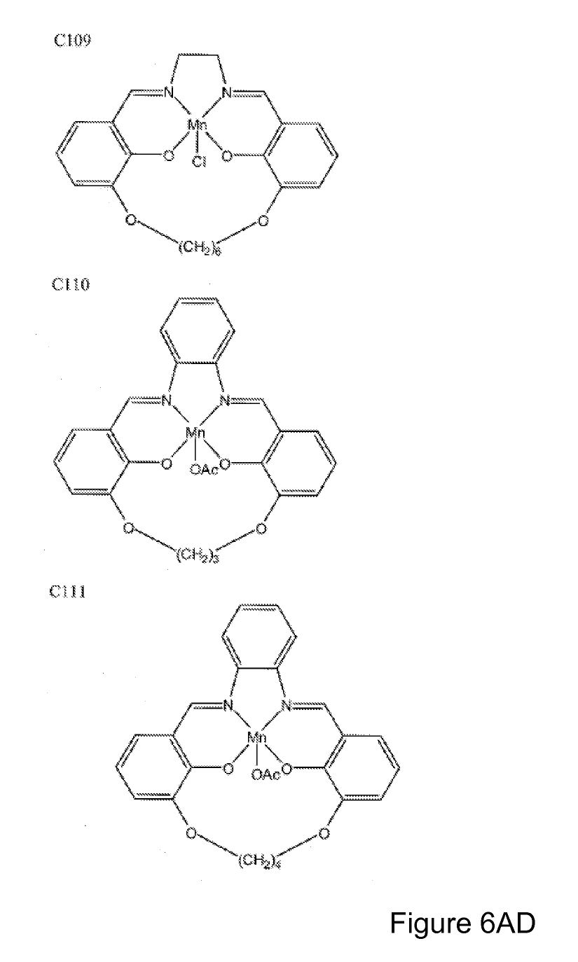

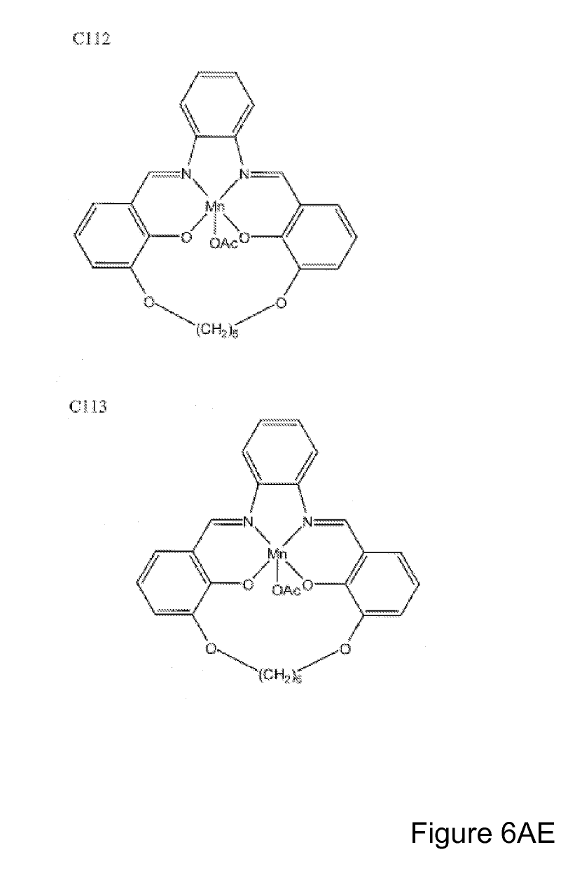

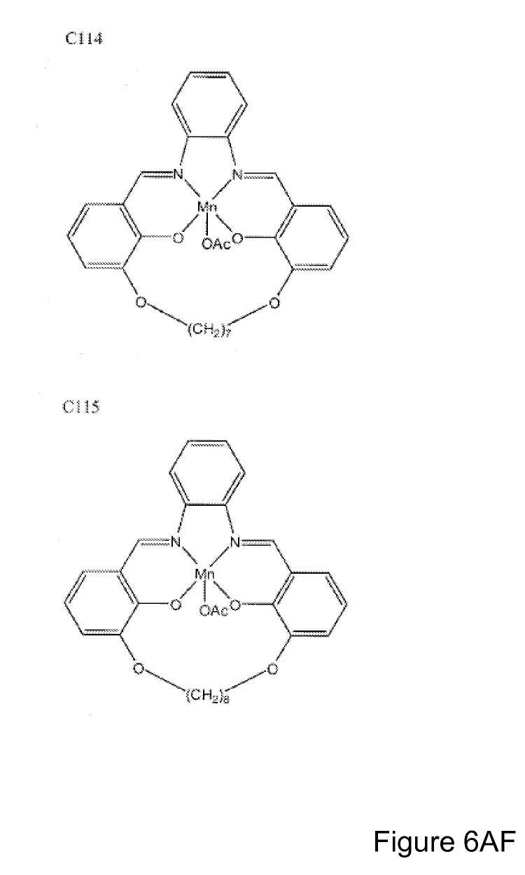

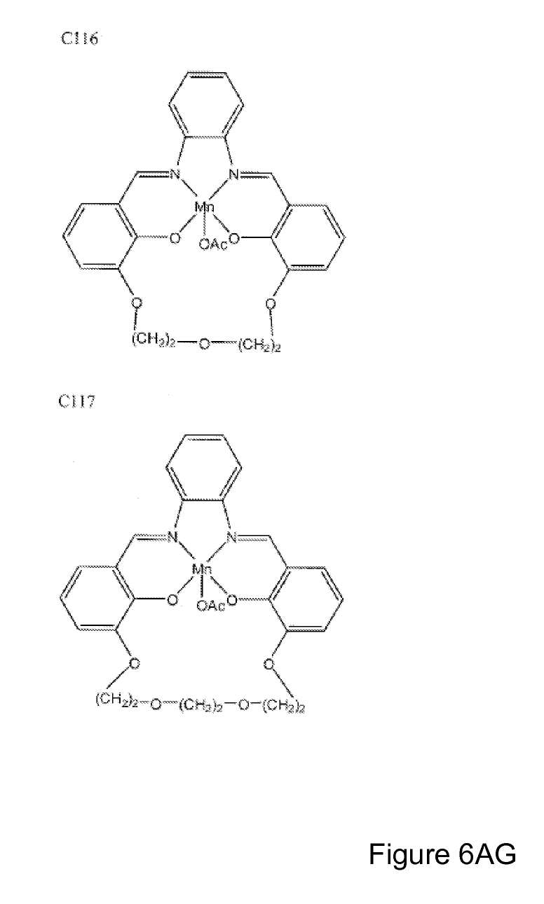

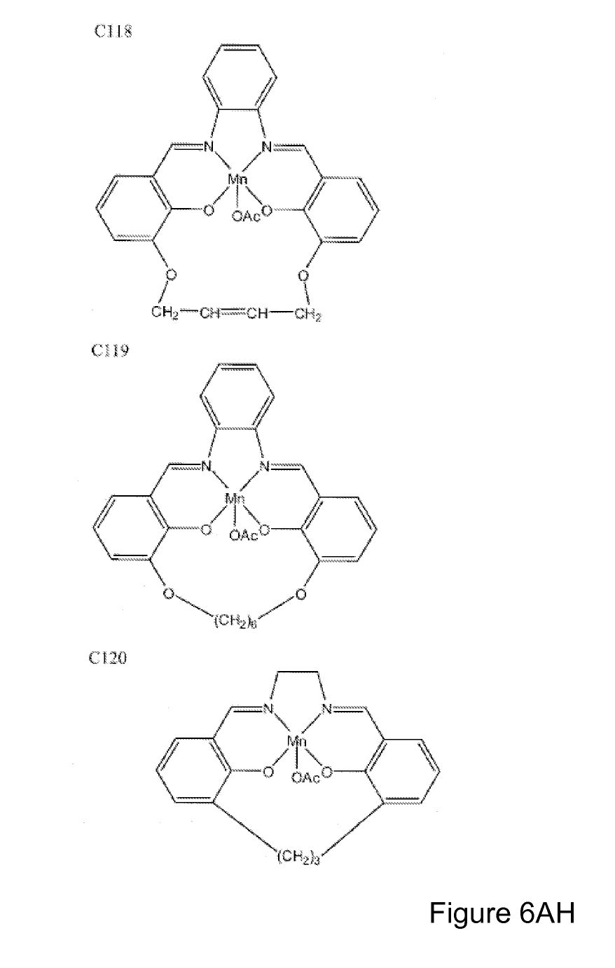

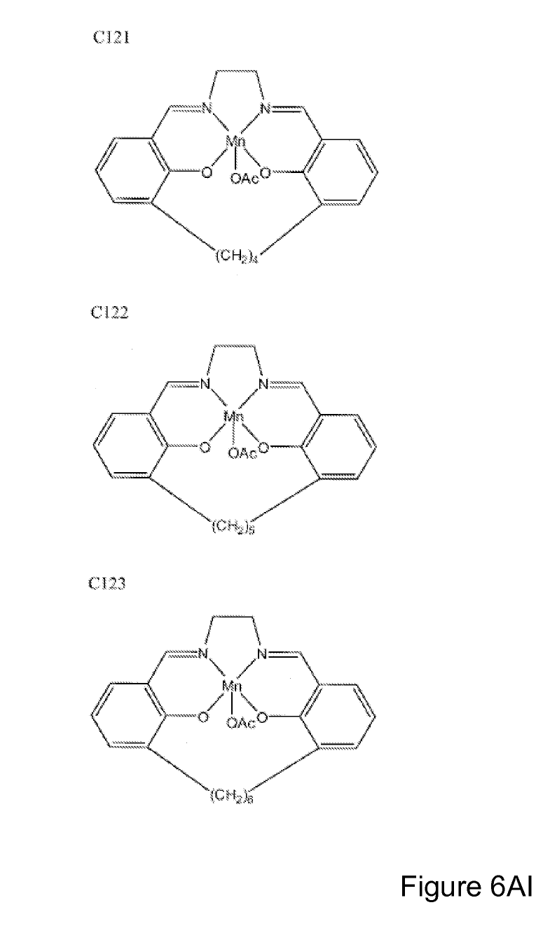

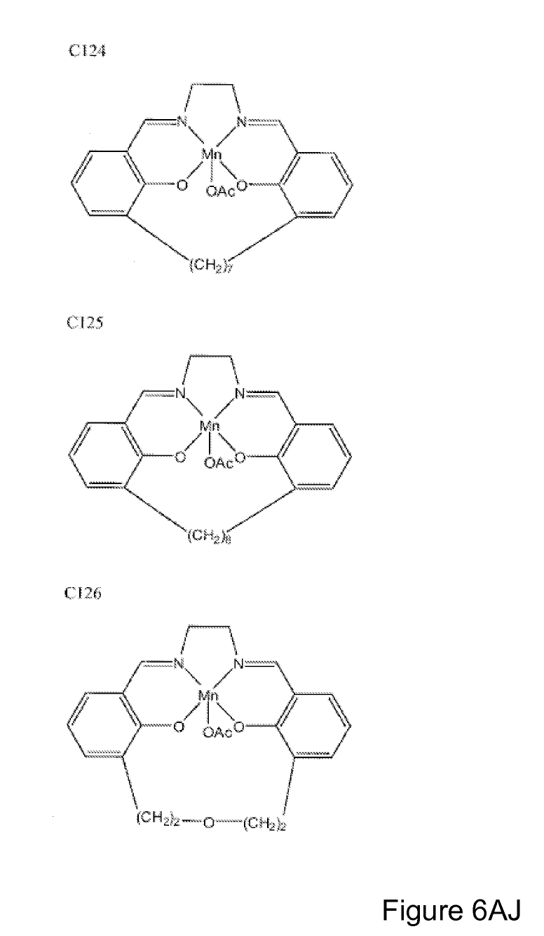

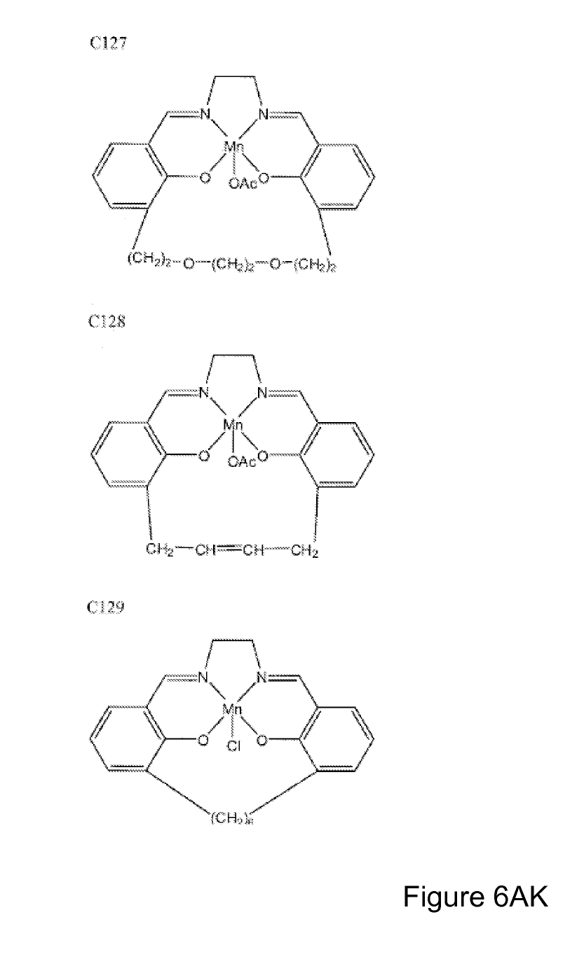

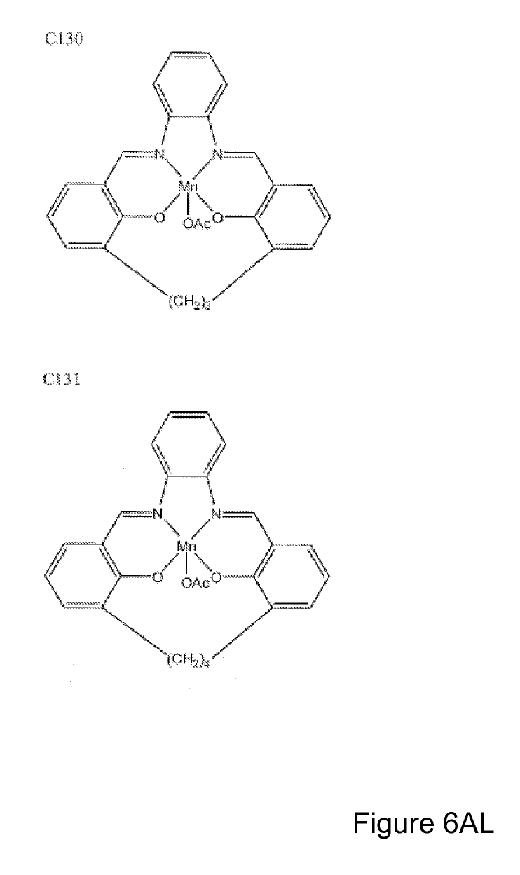

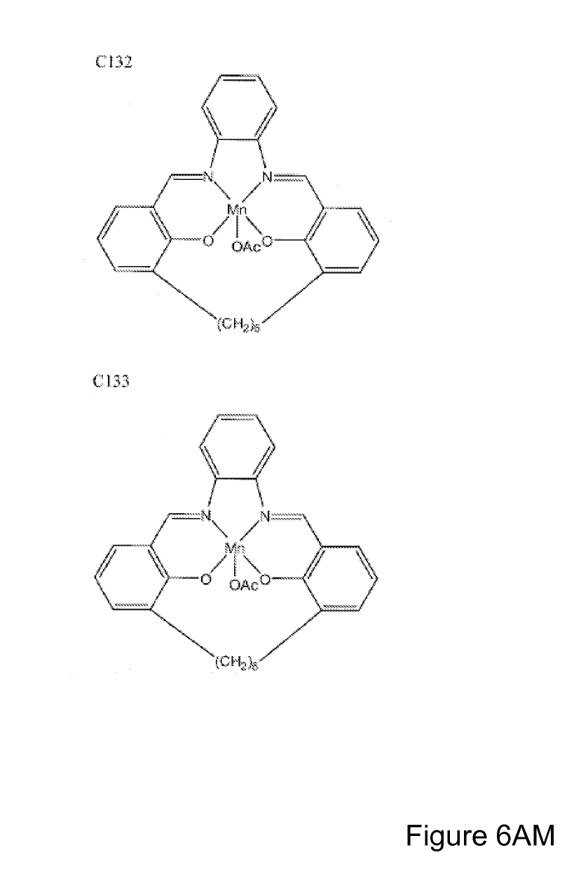

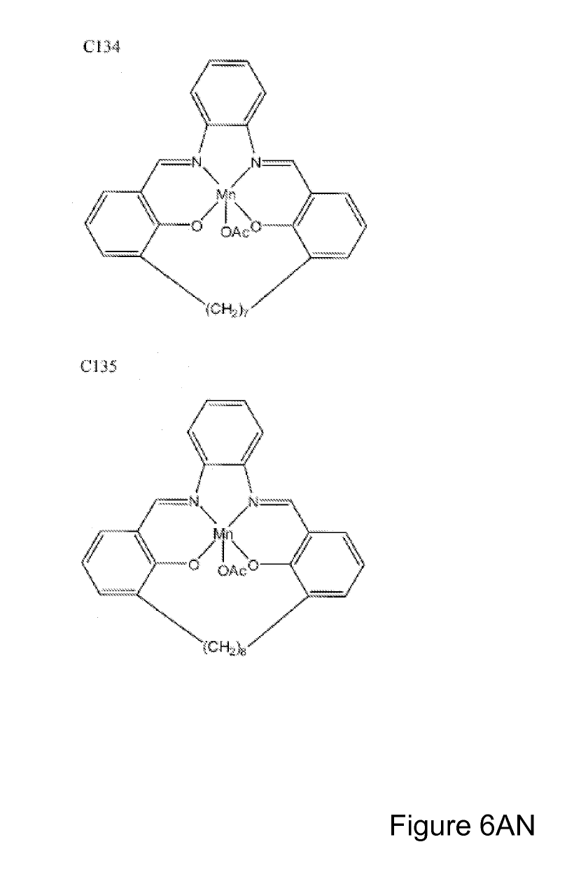

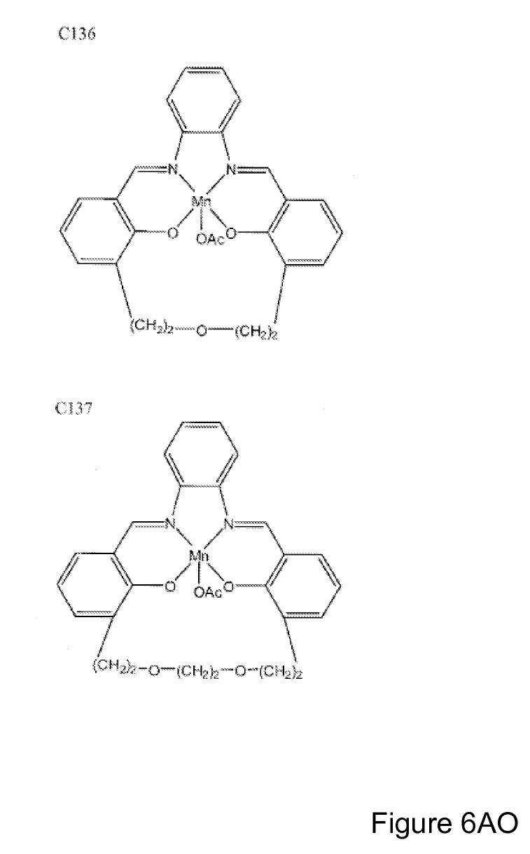

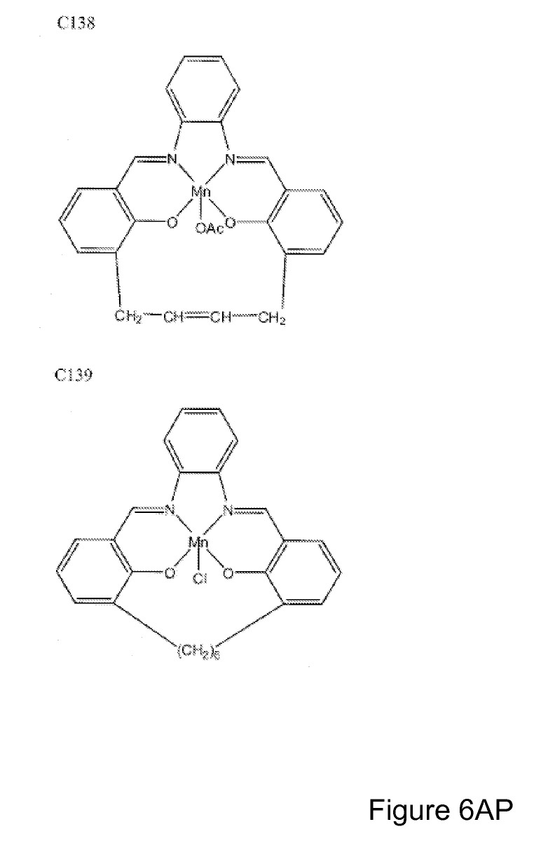

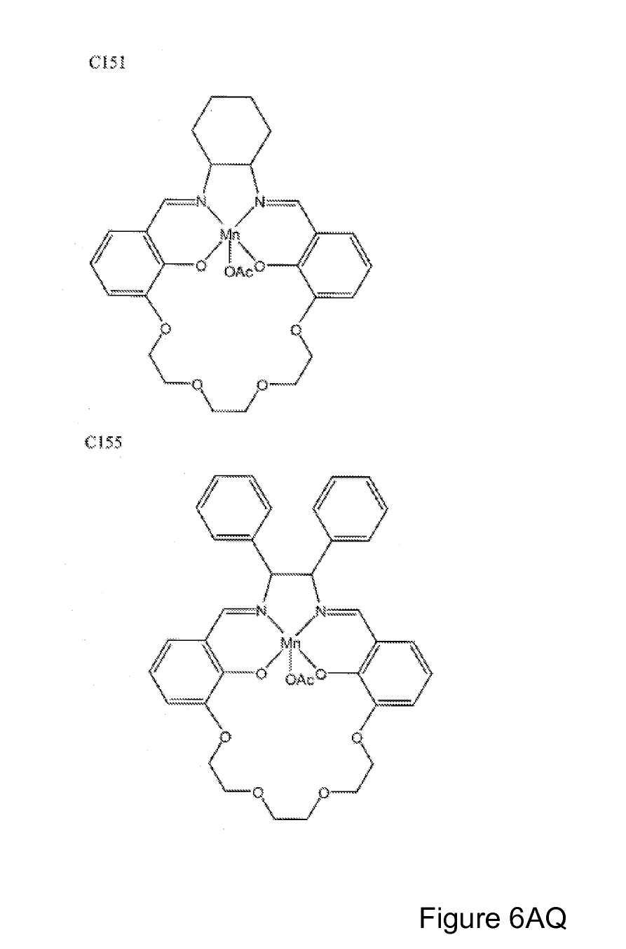

For example, the synthetic SOD/catalase can be a salen-manganese complex such as salen-Mn(III) complex. As used herein, a “salen-transition metal complex” refers to a compound having a structure according to Structure I, Structure II, Structure III, Structure IV, Structure V, Structure VI, Structure VI, Structure VII, Structure VIII, Structure IX (see infra and FIGS. 5A-5D) or any of the structures of compounds C1, C4, C6, C7, C9, C10, C11, C12, C15, C17, C20, C22, C23, C25, C27, C28, C29, C30, C31-C94 as shown in FIGS. 6A-6AA, or the Structures X-XXII as shown in FIGS. 5D-5I. In one embodiment, the SOD/catalase mimetic salen-metal complex according to any embodiment as described herein possesses detectable catalase activity in vitro, for example as shown in Table 1. In another embodiment, the SOD/catalase mimetic salen-metal complex is selected from the group consisting of: C1; C4; C7 (EUK-8); C9; C10; C11; C12; C31; C32; C33; C34; C35; C36; C37; C40 (EUK-134); C41; C42; C43; C44; C45; C46; C47; C48; C49; C50; C51; C52; C67; C68 (EUK-189); C76; C81; C82; C83; C84; and C85.

TABLE 1
Catalase Activity of salen-metal complexes

| Salen-Metal complex | Relative Catalase Rate |
|---|---|
| C9 | 20 |
| C35 | 25 |
| C51 | 25 |
| C84 | 31 |
| C85 | 31 |
| C33 | 38 |
| C83 | 42 |
| C11 | 46 |
| C34 | 46 |
| C52 | 73 |
| C31 | 92 |
| C4 | 100 |
| C7 (EUK-8) | 100 |
| C37 | 117 |
| C41 | 120 |
| C49 | 123 |
| C36 | 128 |
| C1 | 131 |
| C12 | 144 |
| C40 (EUK-134) | 155 |
| C67 | 159 |
| C50 | 174 |
| C10 | 179 |
| C32 | 188 |
| C68 (EUK-189) | 196 |
| C42 | 231 |
| C44 | 272 |
| C47 | 343 |
| C82 | 345 |
| C45 | 357 |
| C76 | 427 |
| C43 | 446 |
| C46 | 465 |
| C48 | 485 |
| C81 | 493 |

*Catalase rate is expressed relative to C7 (EUK-8), wherein the rate for C7 (EUK-8) is set at 100%.

In one example the SOD/catalase mimetic salen-metal complex is selected from the group consisting of: C9; C35; C51; C84; C85; C33; C83; C11; C34; C52; and C31. In another example the SOD/catalase mimetic salen-metal complex is selected from the group consisting of: C4; C7 (EUK-8); C37; C41; C49; C36; C1; C12; C40 (EUK-134); C67; C50; C10; C32; and C68 (EUK-189). In another example the SOD/catalase mimetic salen-metal complex is C42 or C44. In another example the SOD/catalase mimetic salen-metal complex is selected from the group consisting of: C47; C82; and C45. In another example the SOD/catalase mimetic salen-metal complex is selected from the group consisting of: C76; C43; C46; C48; and C81. In some embodiments, the SOD/catalase mimetic salen-metal complex is C7 (EUK-8) or C68 (EUK-189). In other embodiments, the SOD/catalase mimetic salen-metal complex can be C68 (EUK-189).

In another example, the synthetic SOD/catalase mimetic used in the process comprises a 3,3′ bridging group as described e.g., by virtue of Structure XXIII or Structure XXIV of U.S. Pat. No. 6,589,948 or 7,122,537. Some examples of such complexes are Mn(III)-containing complexes C101-C155 as shown in FIGS. 6AA-6AQ hereof, and one embodiment a complex possessing detectable catalase activity such as, for example, a complex selected from the group consisting of C103, C105, C107 (EUK-207), and C117, and in another embodiment the complex is C107 (EUK-207).

In another example, the synthetic SOD/catalase mimetic used in the process comprises a metalloporphyrin as described in U.S. Pat. No. 6,403,788, such as a metalloporphyrin comprising a structure according to Structure XXV-XXXII as shown in FIG. 5K-5O hereof. Some metalloporphyrins possess detectable catalase activity e.g., one or more compounds represented by a sub-generic formula shown in Table 2.

TABLE 2
Catalase Activity of metalloporphyrins

| Metalloporphyrin | Relative Catalase Rate |
|---|---|
| Formula XXV | 54 |
| Formula XXX | 81 |
| Formula XXVII | 0.41 |
| Formula XXVIII | 34 |
| Formula XXXI | 44 |
| Formula XXIX | 215 |

*Catalase rate is expressed relative to C7 (EUK-8), wherein the rate for C7 (EUK-8) is set at 100%.

In one example, a metalloporphyrin compound is represented by a sub-generic formula selected from structures of the group consisting of: XXV; XXX, and XXIX. In one example, a metalloporphyrin compound is represented by a sub-generic formula structure XXVIII or XXXI. In one example, a metalloporphyrin compound is represented by a sub-generic formula XXIX.

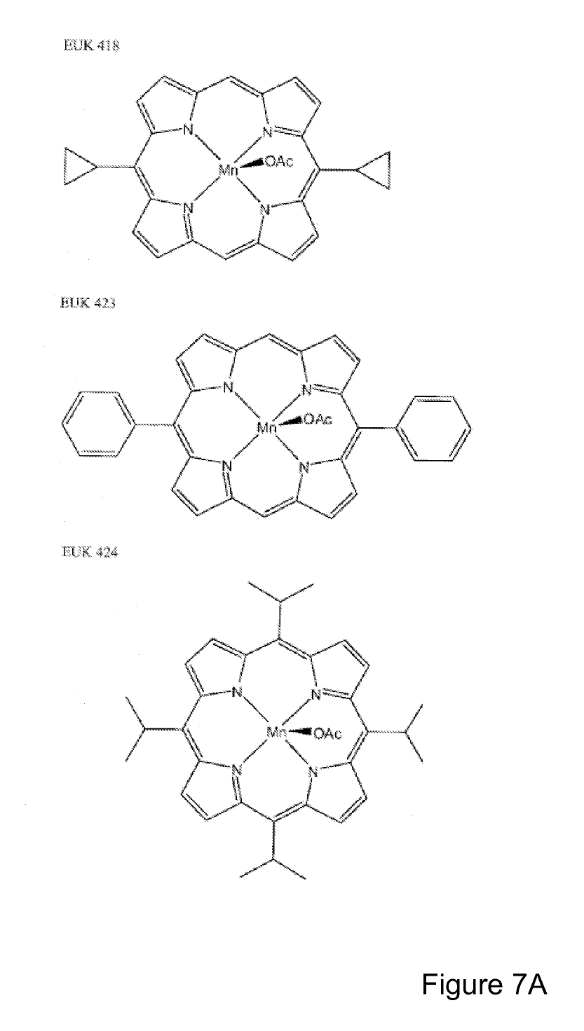

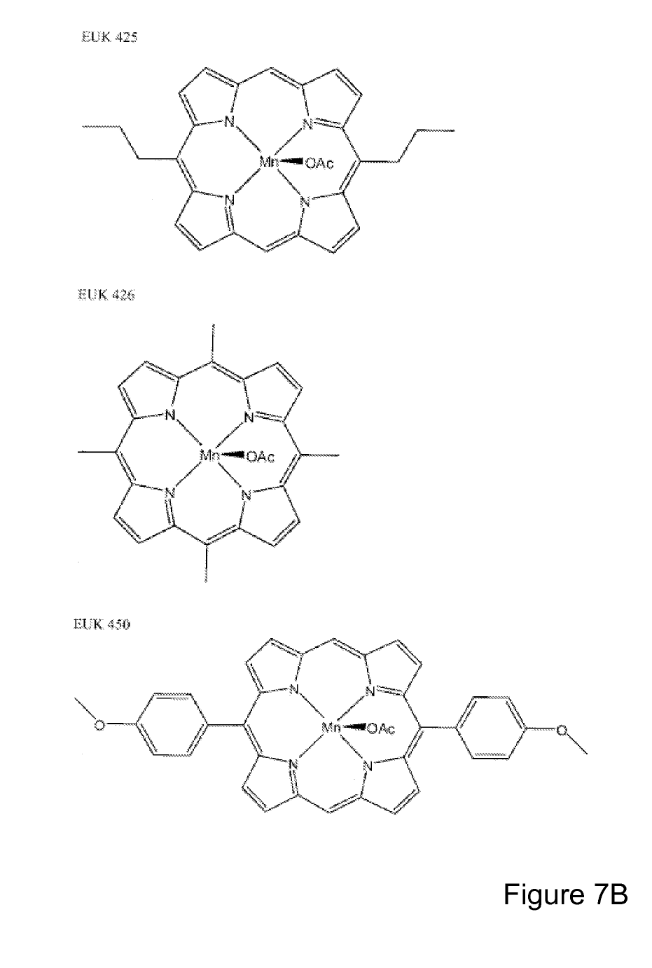

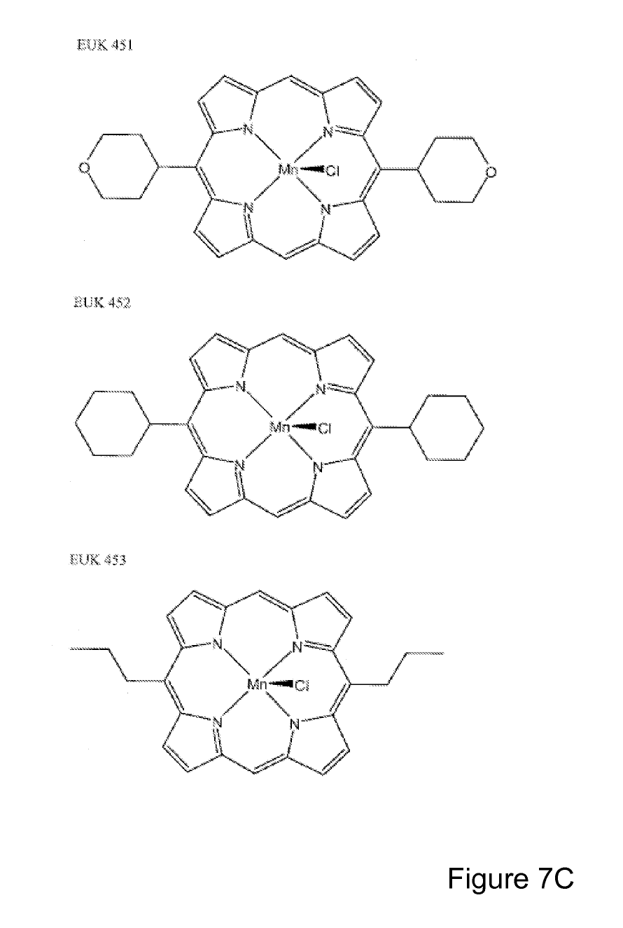

In another example, the synthetic SOD/catalase mimetic used in the process comprises a metalloporphyrin compound e.g., as described in International Patent Publication Nos. WO 2005/000854 or WO 2008/033444. Such compounds may have a structure according to Structure XXXIII, Structure XXXIV, Structure XXXV, Structure XXXVI (see infra and FIGS. 5P-5Q) including, for example, any one or more of the compounds as shown in FIGS. 7A-7C and in some embodiments:
a) {[{(Porphine-5,15-diyl) bis[cyclopropyl-diyl]}] (2-)-N²¹, N²², N²³, N²⁴} manganese(III) acetate (EUK-418);
b) {[{(Porphine-5,15-diyl) bis[benzyl-diyl]}] (2-)-N²¹, N²², N²³, N²⁴} manganese(III) acetate (EUK-423);
c) (5,10,15,20-Tetraisopropylporphyrinato) manganese (III) acetate (EUK-424);
d) (5,10,15,20-Tetraethylporphyrinato) manganese (III) acetate (EUK-425);
e) (5,10,15,20-Tetramethylporphyrinato) manganese (III) acetate (EUK-426);
f) {[{(Porphine-5,15-diyl) bis[benzene-1,4 diyl (4-methyl-oxy)]}](2-)-N²¹, N²², N²³, N²⁴} manganese(III) acetate (EUK-450);
g) {[{(Porphine-5,15-diyl) bis[4-Tetrahydropyrano-diyl]}](2-)-N²¹, N²², N²³, N²⁴} manganese(III) chloride (EUK-451);
h) {[{(Porphine-5,15-diyl) bis[cyclohexyl-diyl]}] (2-)-N²¹, N²², N²³, N²⁴} manganese(III) chloride (EUK-452); or
i) {[{(Porphine-5,15-diyl) bis[propyl-diyl]}] (2-)-N²¹, N²², N²³, N²⁴} manganese(III) chloride (EUK-453).

The metalloporphyrin derivative can also possess detectable catalase activity, for example as shown in Table 3.

TABLE 3
Catalase Activity of orally bioavailable
metalloporphyrin derivatives

| Metalloporphyrin | Relative Catalase Rate |
|---|---|
| EUK-418 | 70 |
| EUK-423 | 56 |
| EUK-425 | 84 |
| EUK-450 | 84 |
| EUK-451 | 140 |
| EUK-452 | 105 |

*Catalase rate is expressed relative to C7 (EUK-8), wherein the rate for C7 (EUK-8) is set at 100%.

Some metalloporphyrin derivatives can be selected from the group consisting EUK-418, EUK-423, EUK-450, EUK-451, and EUK-452, or alternatively, from the group consisting of EUK-418, EUK-423, EUK-425 and EUK-450, or alternatively from the group consisting of EUK-451 and EUK-452. In another example, the metalloporphyrin derivative is EUK-451 or EUK-452.

In another example, the SOD/catalase mimetic is an anti-apoptotic compound that also provides a protective effect against radiation-induced apoptosis and is selected from the group consisting of EUK-418, EUK-423, EUK-450, EUK-451, and EUK-452. Derivative EUK-451 is particularly useful due to its low cytotoxicity and high anti-apoptotic activity.

There are various modes of administering a pharmaceutical formulation in accordance with the process. In one example, the formulation is administered topically including transferral e.g., as ointments, creams, suspensions, lotions, powders, solutions, pastes, gels, sprays, aerosols, oils. In another example, the formulation is administered orally e.g., as capsules, soft gels, or tablets; powders or granules; solutions or suspensions in aqueous or non-aqueous liquids; edible foams or whips; or oil-in-water liquid emulsions or water-in-oil liquid emulsions. In another example, the formulation is administered by injection or infusion e.g., subdermal, intravenous, intraperitoneal, subcutaneous, intramuscular, intra-arterial, intralesional injection. Useful modes of administration permit or facilitate the formulation to reach any layer of the epidermis where there is oxidative damage.

For example, the synthetic SOD/catalase mimetic is a salen-manganese complex such as salen-Mn(III) complex having a structure according to Structure I:

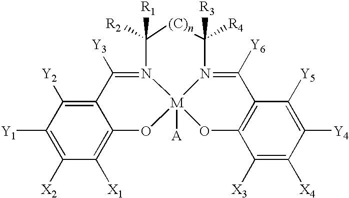

wherein M is a transition metal ion, in one embodiment Mn; A is an axial ligand (anion) and is a halide, acetate, acetyl, acetoxy, ethoxy, formate, formyl, methoxy, PF₆, triflate, tosylate, or is an oxygen atom bound via a double bond to the transition metal (M); A is Cl, Br, F, MeO or OAc; and n is 0, 1, 2, or 6. X₁, X₂, X₃ and X₄ are independently selected from the group consisting of hydrogen, silyls, aryls, arylalkyls, primary alkyls, secondary alkyls, tertiary alkyls, alkoxys, aryloxys, aminos, quaternary amines, heteroatoms, and hydrogen; in one embodiment X₂ and X₃ are from the same functional group, such as hydrogen, ethoxy, methoxy, quaternary amine, or tertiary butyl, and X₂ and X₄ are in one embodiment hydrogen; in other embodiments Xᵢ and X₃ are each F, Cl, Br, OAc, OMe, OH, or H and X₂ and X₄ are each F, Cl, Br, OAc, OMe, OH, or H, in certain embodiments when X₁ and X₃ are other than H, X₂ and X₄ are both H, and vice versa. Y₁, Y₂, Y₃, Y₄, Y₅, and Y₆ are independently selected from the group consisting of hydrogen, halides, alkyls, aryls, arylalkyls, silyl groups, aminos, alkyls or aryls bearing heteroatoms; aryloxys, alkoxys, and halide; in one embodiment, Y₁ and Y₄ are H, alkoxy, halide, or amino groups. In another embodiment, Y₂ and Y₄ are the same. R, R₂, R₃ and R₄ are independently selected from the group consisting of H, CH₃, C₂H₅, C₆H₅, O-benzyl, primary alkyls, fatty acid esters, substituted alkoxyaryls, heteroatom-bearing aromatic groups, arylalkyls, secondary alkyls, and tertiary alkyls.

In another example, the synthetic SOD/catalase mimetic is a salen-manganese complex having a structure according to Structure II:

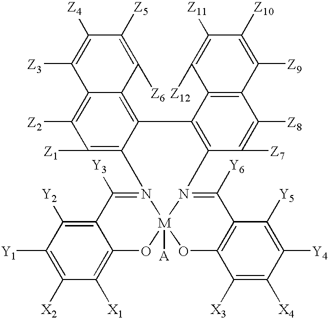

as above M is a transition metal ion, such as Mn, and A is an axial ligand (anion) composed a halide, acetate, acetyl, acetoxy, ethoxy, formate, formyl, methoxy, PF₆, triflate, tosylate, or is an oxygen atom bound via a double bond to the transition metal (M); A is Cl, Br, F, MeO or OAc, in one embodiment Cl; where at least one of X₁ or X₂ is selected from the group consisting of aryls, primary alkyls, secondary alkyls, tertiary alkyls, and heteroatoms or H; where at least one of X₁ or X₃ is selected from the group consisting of aryls, primary alkyls, secondary alkyls, tertiary alkyls, arylalkyls, heteroatoms, and hydrogen, and in some embodiments, X₁ or X₃ is selected from tertiary butyl or hydrogen; and where Y₁, Y₂, Y₃, Y₄, Y₅, Y₆, Z₁, Z₂, Z₃, Z₄, Z₅, Z₆, Z₇, Z₈, Z₉, Z₁₀, Z₁₁, and Z₁₂ are independently selected from the group consisting of hydrogen, halides, alkyls, aryls, amines, alkoxy, substituted alkoxy, arylalkyls, aryloxys, and alkyl groups bearing heteroatoms. In one embodiment, Y₁ and Y₄ are selected from the group consisting of lower alkyls, alkoxy, halide, and amino groups, in another embodiment, Y₁ and Y₄ are selected from the group consisting of methoxy, chloro, and primary amine.

In another example, the synthetic SOD/catalase mimetic is a salen-manganese complex having a structure according to Structure III:

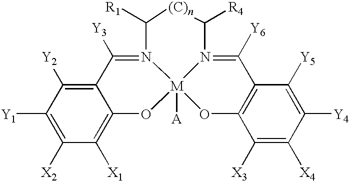

where M is a transition metal ion such as Mn, Mg, Co, Fe, Zn, Cu, V, Cr, and Ni; A is an axial ligand composed of a halide, acetate, formate, PF₆, triflate, tosylate, or is an oxygen atom bound via a double bond to the metal (M); and A can be Cl and M can be Mn; where n is 4, 5, or 6; where X₁, X₂, X₃, and X₄ are independently selected from the group consisting of aryls, arylalkyls, aryloxyss, primary alkyls, secondary alkyls, tertiary alkyls, alkoxy, substituted alkoxy, heteroatoms, aminos, quaternary amines, and hydrogen; in some embodiments, at least one of X₁ or X₃ are selected from the group consisting of aryls, primary alkyls, secondary alkyls, tertiary alkyls, quaternary amines, arylalkyls, heteroatoms, and hydrogen; in one embodiment X₁ and X₃ are identical and are hydrogen, OMe, OAc, F, ethoxy, hydroxy, Br, or tertiary butyl; if X₁ and X₃ are H, then X₂ and X₄ can be selected from the group consisting of aryls, primary alkyls, secondary alkyls, tertiary alkyls, quaternary amines, arylalkyls, heteroatoms, and hydrogen; in some embodiments X₁ and X₄ are identical and are hydrogen, OMe, OAc, F, ethoxy, hydroxy, and Br; Y₁, Y₂, Y₃, Y₄, Y₅, and Y₆ are selected from the group consisting of aryls, arylalkyls, primary alkyls, secondary alkyls, tertiary alkyls, alkoxys, substituted alkoxys, aryloxys, halides, heteroatoms, aminos, quaternary amines, and hydrogen; in one embodiment at least one of Y₁ or Y₄ are selected from the group consisting of aryls, primary alkyls, secondary alkyls, tertiary alkyls, substituted alkoxy, heteroatoms, amines, and halides

In another example, the synthetic SOD/catalase mimetic is a salen-manganese complex having a structure according to Structure IV:

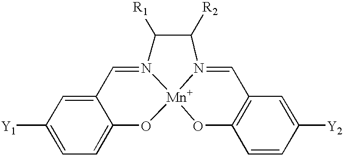

where Y₁ and Y₂ are independently selected from the group consisting of methoxy, ethoxy, methyl, ethyl, formyl, acetyl, t-butyl, chloro, bromo, iodo, fluoro, amino, quaternary amine, alkylamino, dialkylamino, and hydrogen; R₁ and R₂ are independently selected from the group consisting of: phenyl, benzyloxy, chlorobenzyloxy, hydrogen, amino, quaternary amine, or fatty acid ester. In one embodiment, Y₁ and Y₂ are identical.

In another example, the synthetic SOD/catalase mimetic is a salen-manganese complex having a structure according to Structure V:

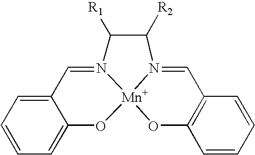

where R₁ and R₂ are selected independently from the group consisting of: phenyl, benzyloxy, chlorobenzyloxy, methoxy, ethoxy, hydrogen, amino, quaternary amine, methoxy, ethoxy, or fatty acid ester. In one embodiment, R₁ and R₂ are identical.

In another example, the synthetic SOD/catalase mimetic is a salen-manganese complex having a structure according to Structure VI:

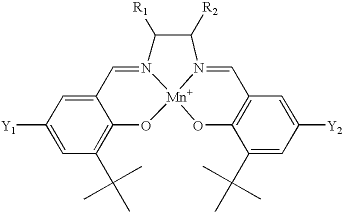

where Y₁ and Y₂ are independently selected from the group consisting of methoxy, ethoxy, methyl, ethyl, t-butyl, chloro, bromo, iodo, amino, quaternary amine, alkylamino, dialkylamino, and hydrogen; R₁ and R₂ are selected independently from the group consisting of: phenyl, benzyloxy, chlorobenzyloxy, hydrogen, amino, quaternary amine, or fatty acid ester. In one embodiment, Y₁ and Y₂ are identical, and R₁ and R₂ are identical.

In another example, the synthetic SOD/catalase mimetic is a salen-manganese complex having a structure according to Structure VII:

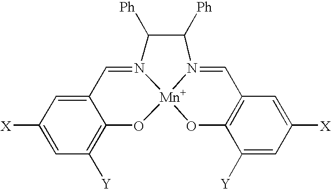

where X is selected from the group consisting of methoxy, ethoxy, methyl, ethyl, formyl, acetyl, t-butyl, chloro, bromo, iodo, fluoro, amino, quaternary amine, alkylamino, dialkylamino, and hydrogen; Y is selected from the group consisting of t-butyl, methoxy, ethoxy, formyl, acetyl, Cl, Br, F, quaternary amine, amino, and hydrogen.

In another example, the synthetic SOD/catalase mimetic is a salen-manganese complex having a structure according to Structure VIII:

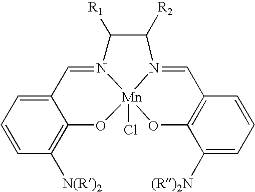

where R₁ and R₂ are independently selected from the group consisting of aryloxys, alkoxys, aryls, and hydrogen; R′ and R″ are independently selected from the group consisting of alkyls, aryls, and hydrogen. In one embodiment, at least one of the amino groups is protonated at physiological pH (i.e., pH 7.3-7.8). In one embodiment, R′ or R″ alkyls can include but are not limited to: methyl, ethyl, and propyl. In one embodiment, R₁ and R₂ aryloxys include but are not limited to benzyloxy and chlorobenzyloxy. In one embodiment, R₁ and R₂ alkoxys include but are not limited to ethoxy and methoxy.

In another example, the synthetic SOD/catalase mimetic is a salen-manganese complex having a structure according to Structure IX:

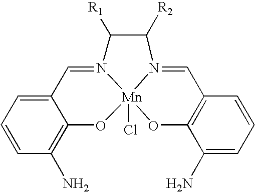

where R is selected from the group consisting of alkyls and hydrogen. In one embodiment, at least one of the amino groups are protonated at physiological pH (i.e., pH 7.3-7.8).

In another example, the synthetic SOD/catalase mimetic is a salen-manganese complex having a structure according to any of the structures of compounds C1, C4, C6, C7, C9, C10, C11, C12, C15, C17, C20, C22, C23, C25, C27, C28, C29, C30, C31-C94 as shown in Figures or the Structures X-XXII as shown in FIGS. 5D through 5I.

In another example, the synthetic SOD/catalase mimetic is a cyclic salen-metal compound having a structure according to Structure XXIII:

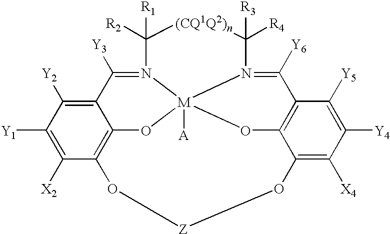

### or Structure XXIV:

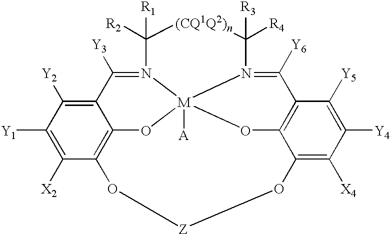

In structures XXIII and XXIV, M is a metal, and can be a transition metal, and A is an anion, and can be a halogen or an organic anion (e.g., acetate). Examples of suitable transition metals include, but are not limited to, Mn, Cr, Fe, Zn, Cu, Ni, Co, Ti, V, Ru and Os. Examples of suitable anions include, but are not limited to, PF6, (Aryl) 4, BF4, B (Aryl) 4, halogen, acetate, acetyl, formyl, formate, triflate, tosylate or, alternatively, the anion can be an oxygen atom bound via a double bond to the metal, i.e., M. X₁ and X₂ are independently selected and are functional groups including, but not limited to, hydrogen, halogen, alkyls, substituted alkyls, aryls, substituted aryls, heterocyclics, substituted heterocyclics, heteroaryls, substituted heteroaryls, silyls, aminos, fatty acid esters, alkoxys, aryloxys and acyloxys. Y₁, Y₂ Y₃, Y₄, Y₅ and Y₆ in Formulae I and II, are independently selected and are functional groups including, but not limited to, hydrogen, halogen, alkyls, substituted alkyls, aryls, substituted aryls, heterocyclics, substituted heterocyclics, heteroaryls, substituted heteroaryls, silyls, aminos, fatty acid esters, alkoxys, aryloxys and acyloxys. R₁, R₂, R₃ and R₄ are independently selected and are functional groups including, but not limited to, hydrogen, halogens, alkyls, substituted alkyls, aryl, substituted aryl, heterocyclics, substituted heterocyclics, heteroaryls, substituted heteroaryls, silyls, aminos, fatty acid esters, alkoxys, aryloxys and acyloxys; with the proviso that one of R₁ or R₂ may be covalently linked to one of R₃ or R₄ forming a cyclic structure. Z, in is a bridging group. Q1 and Q2, are independently selected and are functional groups including, but not limited to, hydrogen, halogen, alkyls, substituted alkyls, aryls, substituted aryls, heterocyclics, substituted heterocyclics, heteroaryls, substituted heteroaryls, silyls, aminos, fatty acid esters, alkoxys, aryloxys and acyloxys. The index “n” is 0, 1 or 2.

In another example, the synthetic SOD/catalase mimetic is a cyclic salen-metal compound having a structure according to any of the structures C101-C155 as shown in FIGS. 6AA-6AQ.

In another example, the synthetic SOD/catalase mimetic used in the process o is a metalloporphyrins having a structure according to Structure XXV:

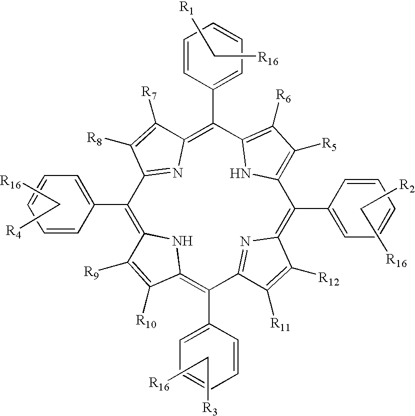

wherein R₁, R₂, R₃ and R₄ are the same or different and are each a group of the formula: where L is a linker of about 2 to about 12 atoms in length. The atoms within the linker are carbon atoms optionally interspersed with from 1 to 4 heteroatoms selected from the group consisting of oxygen, nitrogen and sulfur. In one embodiment, L is a linear C₂-C₆ alkylene group, and in another embodiment ethylene, X is nitrogen or phosphorus; R₁₃, R₁₄ and R₁₅ are each, independently, hydrogen, alkyl or arylalkyl; Y-is a monovalent anion, R₅ R₆, R₇, R₈, R₉, R₁₀, R₁₁ and R₁₂ are each independently selected from the group consisting of hydrogen, alkyl and halo; and each R₁₆ independently represents one or more substituents independently selected from the group consisting of hydrogen, hydroxy, halo and alkyl.

In another example, the synthetic SOD/catalase mimetic used in the process is a metalloporphyrins having a structure according to any of the structures XXVI-XXXII as shown in FIG. 5K-5O.

In another example, the synthetic SOD/catalase mimetic is an orally bioavailable water soluble metalloporphyrin derivative having a structure according to:

### Structure XXXIII:

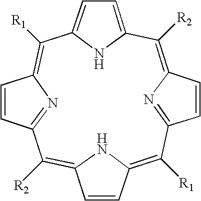

wherein one or both occurrences of R1 is aliphatic or aromatic and wherein one or both occurrences of R2 is hydrogen or aliphatic. This clearly extends to mixtures of such substitutents.

By “aliphatic” is meant a straight-chained, branched or cyclic (non-aromatic) saturated hydrocarbon. Typical straight-chained aliphatic or branched aliphatic groups have from one to about twenty carbon atoms, in one embodiment from one to about ten carbon atoms. Typical cyclic aliphatic groups have from three to about eight ring carbon atoms. Exemplary aliphatic groups include a straight, branched chain or cyclic alkyl group e.g., methyl, ethyl, propyl, n-propyl, iso-propyl, cyclopropyl, n-butyl, iso-butyl, sec-butyl, pentyl, hexyl, cyclohexyl, octyl, cyclooctyl, methyloxy, ethyloxy, propyloxy, tetrahydropyrano, etc. The term “alkyl” refers to a hydrocarbon, including both straight-chained, cycloalkyl, groups.

By “aromatic” is meant benzyl or phenyl or a derivative thereof e.g., benzyloxy, phenoxy, methoxyphenyl, etc. or other aryl (i.e., unsubstituted or substituted aromatic hydrocarbon) substituent, the only requirement being the presence of at least one aromatic ring structure or benzene ring.

In another example, the synthetic SOD/catalase mimetic is an orally bioavailable water soluble metalloporphyrin derivative having a structure according to Structure XXXIV, Structure XXXV, Structure XXXVI as shown in FIGS. 5P-5Q or any of the compounds as shown in FIGS. 7A-7C.

### 2.2 Indications

In one embodiment, the disease and/or systems to be treated are associated with oxidative damage in the skin. The SOD/Catalase mimetics described herein are useful in the process.

Diseases and/or symptoms that may be treated by the process include, but are not limited to: redness; burning, itching, rash, dry-flaking skin, wrinkling, freckling, dilatation of capillaries, irregular pigmentation, liver spot, sun-burn, swelling, blistering, broken skin, eczema, psoriasis, acne, urticaria, dermatitis (e.g., contact dermatitis, atopic dermatitis, seborrheic dermatitis, stasis dermatitis, seborrhoeic dermatitis, allergic contact dermatitis, perioral dermatitis, dermatitis herpetiformis, irritant contact dermatitis, or radiation dermatitis), erythema, ulceration, keratoses, basal cell carcinoma, squamous cell carcinoma, melanoma and combinations thereof.

It will also be understood that symptoms that may be treated by the process also include one or more visible symptoms of skin damage arising from sun exposure e.g., exposure to UV radiation such as UVA, UVB or UVC radiation. In one example, sun exposure is acute, or short term, e.g., 1 to about 24 hours duration, and in one embodiment, at least about 4 hours duration. In another embodiment, the UV radiation comprises UVB and/or UVC. In this case, the subject may present with sun-burn, redness, burning, itching, rash, dry-flaking skin, or skin erythema. In another example, sun exposure is longer term or long-term sun exposure, e.g., one or more exposures of greater than 24 hours duration. In yet another embodiment, long-term sun exposure comprises exposure to UVA radiation e.g., one or more exposures to UVA radiation of greater than 24 hours duration per exposure. In this case, the subject may present with wrinkles, one or more symptoms of photoaging including freckling, dilatation of capillaries and irregular pigmentation (liver spots) and or one or more symptoms of skin cancer such as those associated with basal cell carcinoma, squamous cell carcinoma, or melanoma, or combinations thereof.

It will also be understood that symptoms that may be treated by the process also include one or more visible symptoms skin damage arising from exposure to an environmental pollutant, including but not limited to cigarette smoke, or air pollution generated by automobile and other industrial sources, including photochemical smog e.g., due to presence of nitrous oxide or nitric oxide. In this case, the subject may present with wrinkles, one or more symptoms of photochemical aging including freckling, dilatation of capillaries and irregular pigmentation (liver spots) and any symptoms of skin cancer such as those associated with basal cell carcinoma, squamous cell carcinoma, or melanoma.

It will also be understood that symptoms that may be treated by the process also include one or more visible symptoms skin damage arising from exposure to inflammatory agent(s) and/or allergen(s). In this case, the subject may present with redness, itchiness, urticaria, eczema, psoriasis, acne, or dermatitis.

It will also be understood that symptoms that may be treated by the process also include one or more visible symptoms skin damage arising from exposure to ionizing radiation. In this case, the subject may present with radiation dermatitis, skin erythema, ulceration, open blisters, moist desquamation, or a symptom of skin cancer such as associated with basal cell carcinoma, squamous cell carcinoma, or melanoma.

It will also be understood that symptoms that may be treated by the process also include one or more visible symptoms skin damage arising from exposure to food contaminant(s) and/or food additive(s) and/or food preservative(s) and/or medicinal drug(s) and/or drug(s) of addiction and/or topical formulation such as a cosmetic product and/or stress(es) and/or disease(s).

### 2.4. Dosage and Administration

It will be understood that the SOD/catalase mimetic suitable for the recommendation to a particular patient in the process will be selected based on the diagnosis of oxidative damage in the patient and the activity of the SOD/catalase mimetic. For example, the SOD/catalase mimetic may be chosen based on its in vitro catalase activity as described herein relative to the increase in oxidative damage beyond baseline as determined by any embodiment described herein.

In one example, the SOD/catalase mimetic suitable for recommendation may be chosen according to Table 4:

TABLE 4

| Increase in | Relative | |
|---|---|---|
| oxidative damage | Catalase Rate | Exemplary SOD/Catalase mimetics in this |
| beyond baseline | Range* | range: |
| 0-100 | 20-100 | C9; C35; C51; C84; C85; Formula; XXV; C33; |
| | | C83; Formula; XXX; C11; C34; Formula; XXVIII; |
| | | EUK-423; EUK-418; C52; Formula; XXXI; EUK- |
| | | 425; EUK-450; EUK-453; C31; C4; C7 (EUK-8); |
| 100-200 | 20-100 | C9; C35; C51; C84; C85; Formula; XXV; C33; |
| | | C83; Formula; XXX; C11; C34; Formula; XXVIII; |
| | | EUK-423; EUK-418; C52; Formula; XXXI; EUK- |
| | | 425; EUK-450; EUK-453; C31; C4; C7 (EUK-8); |
| 200-300 | 100-200 | C4; C7 (EUK-8); EUK-452; C37; C41; C49; C36; |
| | | C1; EUK-451; C12; C40 (EUK-134); C67; C50; |
| | | C10; C32; C68 (EUK-189); |
| 300-400 | 100-200 | C4; C7 (EUK-8); EUK-452; C37; C41; C49; C36; |
| | | C1; EUK-451; C12; C40 (EUK-134); C67; C50; |
| | | C10; C32; C68 (EUK-189); |
| 400-500 | 200-300 | Formula XXIX; C42; C44 |
| 500-600 | 200-300 | Formula XXIX; C42; C44 |
| 600-700 | 300-400 | C47; C82; C45 |
| 700-800 | 300-400 | C47; C82; C45 |
| 800-900 | 400-500 | C76; C43; C46; C48; C81 |
| 900-1000 | 400-500 | C76; C43; C46; C48; C81 |

*Catalase rate is expressed relative to C7 (EUK-8), wherein the rate for C7 (EUK-8) is set at 100%.

In another example, the SOD/catalase mimetic suitable for recommendation may be chosen according to Table 5:

TABLE 5

| Increase in | Relative | |
|---|---|---|
| oxidative damage | Catalase Rate | Exemplary SOD/Catalase mimetics in this |
| beyond baseline | Range* | range: |
| 0-50 | 20-100 | C9; C35; C51; C84; C85; Formula; XXV; C33; |
| | | C83; Formula; XXX; C11; C34; Formula; XXVIII; |
| | | EUK-423; EUK-418; C52; Formula; XXXI; EUK- |
| | | 425; EUK-450; EUK-453; C31; C4; C7 (EUK-8); |
| 50-100 | 20-100 | C9; C35; C51; C84; C85; Formula; XXV; C33; |
| | | C83; Formula; XXX; C11; C34; Formula; XXVIII; |
| | | EUK-423; EUK-418; C52; Formula; XXXI; EUK- |
| | | 425; EUK-450; EUK-453; C31; C4; C7 (EUK-8); |
| 100-150 | 100-200 | C4; C7 (EUK-8); EUK-452; C37; C41; C49; C36; |
| | | C1; EUK-451; C12; C40 (EUK-134); C67; C50; |
| | | C10; C32; C68 (EUK-189); |
| 150-200 | 100-200 | C4; C7 (EUK-8); EUK-452; C37; C41; C49; C36; |
| | | C1; EUK-451; C12; C40 (EUK-134); C67; C50; |
| | | C10; C32; C68 (EUK-189); |
| 200-250 | 200-300 | Formula XXIX; C42; C44 |
| 250-300 | 200-300 | Formula XXIX; C42; C44 |
| 300-350 | 300-400 | C47; C82; C45 |
| 350-400 | 300-400 | C47; C82; C45 |
| 400-450 | 400-500 | C76; C43; C46; C48; C81 |
| 450-500 | 400-500 | C76; C43; C46; C48; C81 |

*Catalase rate is expressed relative to C7 (EUK-8), wherein the rate for C7 (EUK-8) is set at 100%.

In another example, the SOD/catalase mimetic suitable for recommendation may be chosen according to Table 6:

TABLE 6

| Increase in | Relative | |
|---|---|---|
| oxidative damage | Catalase Rate | Exemplary SOD/Catalase mimetics in this |
| beyond baseline | Range* | range: |
| 0-10 | 20-100 | C9; C35; C51; C84; C85; Formula; XXV; C33; |
| | | C83; Formula; XXX; C11; C34; Formula; XXVIII; |
| | | EUK-423; EUK-418; C52; Formula; XXXI; EUK- |
| | | 425; EUK-450; EUK-453; C31; C4; C7 (EUK-8); |
| 10-20 | 20-100 | C9; C35; C51; C84; C85; Formula; XXV; C33; |
| | | C83; Formula; XXX; C11; C34; Formula; XXVIII; |
| | | EUK-423; EUK-418; C52; Formula; XXXI; EUK- |
| | | 425; EUK-450; EUK-453; C31; C4; C7 (EUK-8); |
| 20-30 | 100-200 | C4; C7 (EUK-8); EUK-452; C37; C41; C49; C36; |
| | | C1; EUK-451; C12; C40 (EUK-134); C67; C50; |
| | | C10; C32; C68 (EUK-189); |
| 30-40 | 100-200 | C4; C7 (EUK-8); EUK-452; C37; C41; C49; C36; |
| | | C1; EUK-451; C12; C40 (EUK-134); C67; C50; |
| | | C10; C32; C68 (EUK-189); |
| 40-50 | 200-300 | Formula XXIX; C42; C44 |
| 50-60 | 200-300 | Formula XXIX; C42; C44 |
| 60-70 | 300-400 | C47; C82; C45 |
| 70-80 | 300-400 | C47; C82; C45 |
| 80-90 | 400-500 | C76; C43; C46; C48; C81 |
| 90-100 | 400-500 | C76; C43; C46; C48; C81 |

*Catalase rate is expressed relative to C7 (EUK-8), wherein the rate for C7 (EUK-8) is set at 100%.

In another example, the SOD/catalase mimetic suitable for recommendation may be chosen according to Table 7:

TABLE 7

| Increase in | Relative | |
|---|---|---|
| oxidative damage | Catalase Rate | Exemplary SOD/Catalase mimetics in this |
| beyond baseline | Range* | range: |
| 0-100 | 20-50 | C9; C35; C51; C84; C85; Formula; XXV; C33; C83; |
| | | Formula; XXX; C11; C34 |
| 100-200 | 20-50 | C9; C35; C51; C84; C85; Formula; XXV; C33; |
| | | C83; Formula; XXX; C11; C34 |
| 200-300 | 50-100 | Formula; XXVIII; EUK-423; EUK-418; C52; |
| | | Formula; XXXI; EUK-425; EUK-450; EUK-453; |
| | | C31; C4; C7 (EUK-8) |
| 300-400 | 50-100 | Formula; XXVIII; EUK-423; EUK-418; C52; |
| | | Formula; XXXI; EUK-425; EUK-450; EUK-453; |
| | | C31; C4; C7 (EUK-8) |
| 400-500 | 100-150 | C4; C7 (EUK-8); EUK-452; C37; C41; C49; C36; |
| | | C1; EUK-451; C12; |
| 500-600 | 150-200 | C40 (EUK-134); C67; C50; C10; C32; C68 |
| | | (EUK-189); |
| 600-700 | 200-250 | Formula XXIX; C42; |
| 700-800 | 250-300 | C44; |
| 800-900 | 350-400 | C47; C82; C45; |
| 900-1000 | 450-500 | C76; C43; C46; C48; C81 |

*Catalase rate is expressed relative to C7 (EUK-8), wherein the rate for C7 (EUK-8) is set at 100%.

In another example, the SOD/catalase mimetic suitable for recommendation may be chosen according to Table 8:

TABLE 8

| Increase in | Relative | |
|---|---|---|
| oxidative damage | Catalase Rate | Exemplary SOD/Catalase mimetics in this |
| beyond baseline | Range* | range: |
| 0-50 | 20-50 | C9; C35; C51; C84; C85; Formula; XXV; C33; C83; |
| | | Formula; XXX; C11; C34 |
| 50-100 | 20-50 | C9; C35; C51; C84; C85; Formula; XXV; C33; |
| | | C83; Formula; XXX; C11; C34 |
| 100-150 | 50-100 | Formula; XXVIII; EUK-423; EUK-418; C52; |
| | | Formula; XXXI; EUK-425; EUK-450; EUK-453; |
| | | C31; C4; C7 (EUK-8) |
| 150-200 | 50-100 | Formula; XXVIII; EUK-423; EUK-418; C52; |
| | | Formula; XXXI; EUK-425; EUK-450; EUK-453; |
| | | C31; C4; C7 (EUK-8) |
| 200-250 | 100-150 | C4; C7 (EUK-8); EUK-452; C37; C41; C49; C36; |
| | | C1; EUK-451; C12; |
| 250-300 | 150-200 | C40 (EUK-134); C67; C50; C10; C32; C68 |
| | | (EUK-189); |
| 300-350 | 200-250 | Formula XXIX; C42; |
| 350-400 | 250-300 | C44; |
| 400-450 | 350-400 | C47; C82; C45; |
| 450-500 | 450-500 | C76; C43; C46; C48; C81 |

*Catalase rate is expressed relative to C7 (EUK-8), wherein the rate for C7 (EUK-8) is set at 100%.

In another example, the SOD/catalase mimetic suitable for recommendation may be chosen according to Table 9:

TABLE 9

| Increase in | Relative | |
|---|---|---|
| oxidative damage | Catalase Rate | Exemplary SOD/Catalase mimetics in this |
| beyond baseline | Range* | range: |
| 0-10 | 20-50 | C9; C35; C51; C84; C85; Formula; XXV; C33; C83; |
| | | Formula; XXX; C11; C34 |
| 10-20 | 20-50 | C9; C35; C51; C84; C85; Formula; XXV; C33; |
| | | C83; Formula; XXX; C11; C34 |
| 20-30 | 50-100 | Formula; XXVIII; EUK-423; EUK-418; C52; |
| | | Formula; XXXI; EUK-425; EUK-450; EUK-453; |
| | | C31; C4; C7 (EUK-8) |
| 30-40 | 50-100 | Formula; XXVIII; EUK-423; EUK-418; C52; |
| | | Formula; XXXI; EUK-425; EUK-450; EUK-453; |
| | | C31; C4; C7 (EUK-8) |
| 40-50 | 100-150 | C4; C7 (EUK-8); EUK-452; C37; C41; C49; C36; |
| | | C1; EUK-451; C12; |
| 50-60 | 150-200 | C40 (EUK-134); C67; C50; C10; C32; C68 |
| | | (EUK-189); |
| 60-70 | 200-250 | Formula XXIX; C42; |
| 70-80 | 250-300 | C44; |
| 80-90 | 350-400 | C47; C82; C45; |
| 90-100 | 450-500 | C76; C43; C46; C48; C81 |

*Catalase rate is expressed relative to C7 (EUK-8), wherein the rate for C7 (EUK-8) is set at 100%.

In another example, the SOD/catalase mimetic suitable for recommendation may be chosen according to Table 10:

TABLE 10

| Increase in | Relative | |
|---|---|---|
| oxidative damage | Catalase Rate | Exemplary SOD/Catalase mimetics in this |
| beyond baseline | Range* | range: |
| 0-100 | 20-35 | C9; C35; C51; C84; C85; Formula XXV; |
| 100-200 | 35-50 | C33; C83; Formula; XXX; C11; C34 |
| 200-300 | 50-80 | Formula XXVIII; EUK-423; EUK-418; C52; |
| | | Formula XXXI; |
| 300-400 | 80-95 | Formula XXXI; EUK-425; EUK-450; EUK-453; C31; |
| 400-500 | 95-125 | C4; C7 (EUK-8); EUK-452; C37; C41; C49; |
| 500-600 | 125-140 | C36; C1; EUK-451; |
| 600-700 | 140-185 | C12; C40 (EUK-134); C67; C50; C10; |
| 700-800 | 185-215 | C32; C68 (EUK-189); Formula XXIX; |
| 800-900 | 215-230 | C42 |
| 900-1000 | 230-245 | C42 |

*Catalase rate is expressed relative to C7 (EUK-8), wherein the rate for C7 (EUK-8) is set at 100%.

In another example, the SOD/catalase mimetic suitable for recommendation may be chosen according to Table 11:

TABLE 11

| Increase in | Relative | |
|---|---|---|
| oxidative damage | Catalase Rate | Exemplary SOD/Catalase mimetics in this |
| beyond baseline | Range* | range: |
| 0-50 | 20-35 | C9; C35; C51; C84; C85; Formula XXV; |
| 50-100 | 35-50 | C33; C83; Formula; XXX; C11; C34 |
| 100-150 | 50-80 | Formula XXVIII; EUK-423; EUK-418; C52; |
| | | Formula XXXI; |
| 150-200 | 80-95 | Formula XXXI; EUK-425; EUK-450; EUK-453; C31; |
| 200-250 | 95-125 | C4; C7 (EUK-8); EUK-452; C37; C41; C49; |
| 250-300 | 125-140 | C36; C1; EUK-451; |
| 300-350 | 140-185 | C12; C40 (EUK-134); C67; C50; C10; |
| 350-400 | 185-215 | C32; C68 (EUK-189); Formula XXIX; |
| 400-450 | 215-230 | C42 |
| 450-500 | 230-245 | C42 |

*Catalase rate is expressed relative to C7 (EUK-8), wherein the rate for C7 (EUK-8) is set at 100%.

In another example, the SOD/catalase mimetic suitable for recommendation may be chosen according to Table 12:

TABLE 12

| Increase in | Relative | |
|---|---|---|
| oxidative damage | Catalase Rate | Exemplary SOD/Catalase mimetics in this |
| beyond baseline | Range* | range: |
| 0-10 | 20-35 | C9; C35; C51; C84; C85; Formula XXV; |
| 10-20 | 35-50 | C33; C83; Formula; XXX; C11; C34 |
| 20-30 | 50-80 | Formula XXVIII; EUK-423; EUK-418; C52; |
| | | Formula XXXI; |
| 30-40 | 80-95 | Formula XXXI; EUK-425; EUK-450; EUK-453; C31; |
| 40-50 | 95-125 | C4; C7 (EUK-8); EUK-452; C37; C41; C49; |
| 50-60 | 125-140 | C36; C1; EUK-451; |
| 60-70 | 140-185 | C12; C40 (EUK-134); C67; C50; C10; |
| 70-80 | 185-215 | C32; C68 (EUK-189); Formula XXIX; |
| 80-90 | 215-230 | C42 |
| 90-100 | 230-245 | C42 |

*Catalase rate is expressed relative to C7 (EUK-8), wherein the rate for C7 (EUK-8) is set at 100%.

In another example, the SOD/catalase mimetic suitable for recommendation may be chosen according to Table 13:

TABLE 13

| Increase in | Relative | |
|---|---|---|
| oxidative damage | Catalase Rate | Exemplary SOD/Catalase mimetics |
| beyond baseline | Range* | in this range: |
| 0-100 | 20-30 | C9; C35; C51; C84; C85; |
| 100-200 | 30-40 | C84; C85; Formula XXV; C33; |
| 200-300 | 40-50 | C83; Formula; XXX; C11; C34; |
| 300-400 | 50-60 | Formula XXVIII; EUK-423; |
| 400-500 | 60-70 | EUK-418; C52; Formula XXXI; |
| 500-600 | 70-80 | Formula XXXI; EUK-425; EUK-450; |
| | | EUK-453; |
| 600-700 | 80-90 | C31; |
| 700-800 | 90-100 | C4; C7 (EUK-8); |

*Catalase rate is expressed relative to C7 (EUK-8), wherein the rate for C7 (EUK-8) is set at 100%.

In another example, the SOD/catalase mimetic suitable for recommendation may be chosen according to Table 14:

TABLE 14

| Increase in | Relative | |
|---|---|---|
| oxidative damage | Catalase Rate | Exemplary SOD/Catalase mimetics in |
| beyond baseline | Range* | this range: |
| 0-50 | 20-30 | C9; C35; C51; C84; C85; |
| 50-100 | 30-40 | C84; C85; Formula XXV; C33; |
| 100-150 | 40-50 | C83; Formula; XXX; C11; C34; |
| 150-200 | 50-60 | Formula XXVIII; EUK-423; |
| 200-250 | 60-70 | EUK-418; C52; Formula XXXI; |
| 250-300 | 70-80 | Formula XXXI; EUK-425; EUK-450; |
| | | EUK-453; |
| 300-350 | 80-90 | C31; |
| 350-400 | 90-100 | C4; C7 (EUK-8); |

*Catalase rate is expressed relative to C7 (EUK-8), wherein the rate for C7 (EUK-8) is set at 100%.

In another example, the SOD/catalase mimetic suitable for recommendation may be chosen according to Table 15:

TABLE 15

| Increase in | Relative | |
|---|---|---|
| oxidative damage | Catalase Rate | Exemplary SOD/Catalase mimetics in |
| beyond baseline | Range* | this range: |
| 0-10 | 20-30 | C9; C35; C51; C84; C85; |
| 10-20 | 30-40 | C84; C85; Formula XXV; C33; |
| 20-30 | 40-50 | C83; Formula; XXX; C11; C34; |
| 30-40 | 50-60 | Formula XXVIII; EUK-423; |
| 40-50 | 60-70 | EUK-418; C52; Formula XXXI; |
| 50-60 | 70-80 | Formula XXXI; EUK-425; EUK-450; |
| | | EUK-453; |
| 60-70 | 80-90 | C31; |
| 70-80 | 90-100 | C4; C7 (EUK-8); |

*Catalase rate is expressed relative to C7 (EUK-8), wherein the rate for C7 (EUK-8) is set at 100%.

In another example, the SOD/catalase mimetic suitable for recommendation may be chosen according to Table 16:

TABLE 16

| Increase in | Relative | |
|---|---|---|
| oxidative damage | Catalase Rate | Exemplary SOD/Catalase mimetics in |
| beyond baseline | Range* | this range: |
| 0-100 | 100-110 | C4; C7 (EUK-8); EUK-452; |
| 100-200 | 110-120 | C37; C41; |
| 200-300 | 120-130 | C49; C36; C1; |
| 300-400 | 130-140 | EUK-451; C12; |
| 400-500 | 140-150 | C40 (EUK-134); |
| 500-600 | 150-160 | C40 (EUK-134); C67; |
| 600-700 | 160-170 | C67; |
| 700-800 | 170-180 | C50; C10; |
| 800-900 | 180-190 | C10; C32; |
| 900-1000 | 190-200 | C68 (EUK-189); |

*Catalase rate is expressed relative to C7 (EUK-8), wherein the rate for C7 (EUK-8) is set at 100%.

In another example, the SOD/catalase mimetic suitable for recommendation may be chosen according to Table 17:

TABLE 17

| Increase in | Relative | |
|---|---|---|
| oxidative damage | Catalase Rate | Exemplary SOD/Catalase mimetics in |
| beyond baseline | Range* | this range: |
| 0-50 | 100-110 | C4; C7 (EUK-8); EUK-452; |
| 50-100 | 110-120 | C37; C41; |
| 100-150 | 120-130 | C49; C36; C1; |
| 150-200 | 130-140 | EUK-451; C12; |
| 200-250 | 140-150 | C40 (EUK-134); |
| 250-300 | 150-160 | C40 (EUK-134); C67; |
| 300-350 | 160-170 | C67; |
| 350-400 | 170-180 | C50; C10; |
| 400-450 | 180-190 | C10; C32; |
| 450-500 | 190-200 | C68 (EUK-189); |

*Catalase rate is expressed relative to C7 (EUK-8), wherein the rate for C7 (EUK-8) is set at 100%.

In another example, the SOD/catalase mimetic suitable for recommendation may be chosen according to Table 18:

TABLE 18

| Increase in | Relative | |
|---|---|---|
| oxidative damage | Catalase Rate | Exemplary SOD/Catalase mimetics in |
| beyond baseline | Range* | this range: |
| 0-10 | 100-110 | C4; C7 (EUK-8); EUK-452; |
| 10-20 | 110-120 | C37; C41; |
| 20-30 | 120-130 | C49; C36; C1; |
| 30-40 | 130-140 | EUK-451; C12; |
| 40-50 | 140-150 | C40 (EUK-134); |
| 50-60 | 150-160 | C40 (EUK-134); C67; |
| 60-70 | 160-170 | C67; |
| 70-80 | 170-180 | C50; C10; |
| 80-90 | 180-190 | C10; C32; |
| 90-100 | 190-200 | C68 (EUK-189); |

*Catalase rate is expressed relative to C7 (EUK-8), wherein the rate for C7 (EUK-8) is set at 100%.

In another example, any SOD/catalase mimetic suitable for recommendation as described according to any embodiment herein may be adjusted according to dose. Determination of the appropriate dose is made by a person skilled in the art based on the diagnosis of oxidative skin damage according the process and may also use other parameters or factors known or suspected in the art to affect treatment or predicted to affect treatment. Generally, the dose begins with an amount somewhat less than the optimum dose and is increased by small increments thereafter until the desired or optimum effect is achieved relative to any negative side effects. Important diagnostic measures includes monitoring the efficacy of treatment according to any embodiment as described herein in addition to those of symptoms of the disease and/or disorder being treated, wherein a percentage of efficacy is determined by taking the level of oxidative skin damage after application of an individually tailored pharmaceutical formulation of the process relative to the oxidative damage as determined prior to the application of the tailored pharmaceutical formulation of the method. It is to be understood that based on the percentage efficacy, the individually tailored pharmaceutical formulation may then altered by amending the amount and/or type of SOD/Catalase mimetic present in the pharmaceutical formulation.

An effective amount of therapeutic will reduce, prevent or delay skin damage associated with oxidative damage in the skin as determined by the process according to any embodiment as described herein. The decrease in disease symptoms, for example, as described supra, can be by at least about 10%; usually by at least about 20%; in one embodiment at least about 30%; in another embodiment at least about 40%, and in yet another embodiment by at least about 50%.

Accordingly, selecting an administration regimen for making a recommendation according to the process may also take into consideration several factors, including the serum or tissue turnover rate of the entity, the level of symptoms, the immunogenicity of the entity, and the accessibility of the target cells in the biological matrix. In one embodiment, an administration regimen maximizes the amount of therapeutic SOD/catalase mimetic delivered to the patient consistent with an acceptable level of side effects. Accordingly, the amount of composition delivered depends in part on the particular entity and the severity of the condition being treated.

An effective amount of a SOD/catalase mimetic for a particular patient may also vary depending on factors such as the condition being treated, the overall health of the patient, the method route and dose of administration and the severity of side affects, see, e.g., Maynard, et al. (1996) A Handbook of SOPs for Good Clinical Practice, Interpharm Press, Boca Raton, Fla.; or Dent (2001) Good Laboratory and Good Clinical Practice, Urch Publ., London, UK.

It will be understood that modes of administration according to any embodiment described herein permit or facilitate the formulation to reach any layer of the epidermis where there is oxidative damage. The route of administration is by, e.g., topical or cutaneous application, injection or infusion by intravenous, intraperitoneal, intracerebral, intramuscular, intraocular, intraarterial, intracerebrospinal, intralesional, or pulmonary routes, or by sustained release systems or an implant (see, e.g., Sidman et al. Biopolymers 22:547-556, 1983; Langer, et al. J. Biomed. Mater. Res. 15:167-277, 1981; Langer Chem. Tech. 12:98-105, 1982; Epstein, et al. Proc. Natl. Acad. Sci. USA 82:3688-3692, 1985; Hwang, et al. Proc. Natl. Acad. Sci. USA 77:4030-4034, 1980; U.S. Pat. Nos. 6,350,466 and 6,316,024).

A SOD/catalase mimetic can be provided, for example, by continuous infusion, or by doses at intervals of, e.g., one day, one week, or 1-7 times per week. Doses of a composition may be provided, topically, orally, subdermal, intravenous, intraperitoneal, subcutaneous, intramuscular, intraarterial, intralesional injection. A dose protocol is one involving the maximal dose or dose frequency that avoids significant undesirable side effects. A total weekly dose depends on the type and activity of the SOD/catalase mimetic being used. For example, such a dose is at least about 0.05 ng/kg body weight, or at least about 0.2 ng/kg, or at least about 0.5 ng/kg, or at least about 1 ng/kg, or at least about 10 ng/kg, or at least about 100 ng/kg, or at least about 0.2 mg/kg, or at least about 1.0 mg/kg, or at least about 2.0 mg/kg, or at least about 10 mg/kg, or at least about 25 mg/kg, or at least about 50 mg/kg (see, e.g., Yang, et al. New Engl. J. Med. 349:427-434, 2003; or Herold, et al. New Engl. J. Med. 346:1692-1698, 2002).

### 2.4. Formulations

There are various modes of recommending a pharmaceutical formulation in accordance with the process of the present methods including modes of administration.

Formulation of a pharmaceutical SOD/catalase mimetic will vary according to the route of administration selected (e.g., solution, emulsion, capsule). For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils, for instance. Intravenous vehicles can include various additives, preservatives, or fluid, nutrient or electrolyte replenishers and the like (See, generally, Remington's Pharmaceutical Sciences, 17th Edition, Mack Publishing Co., Pa., 1985).

Pharmaceutical formulations can be adapted for administration by any appropriate route, for example by the topical including transferal, oral (including buccal or sublingual), or parenteral (including subcutaneous, intramuscular, intravenous or intradermal) route. Such formulations can be prepared by any method known in the art of pharmacy, for example by bringing into association the active ingredient with the carrier(s), diluent(s) or excipient(s).

To prepare such pharmaceutical formulations, one or more SOD/catalase mimetics of the present methods and selected on the basis of the diagnosis of skin damage in a subject according to the process is/are mixed with a pharmaceutically acceptable carrier or excipient for example, by mixing with physiologically acceptable carriers, excipients, or stabilizers in the form of, e.g., lyophilized powders, slurries, aqueous solutions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

As will be apparent to a skilled artisan, a SOD/catalase mimetic that is capable of catalysing the conversion of H₂0₂ to H₂0 and catalyses this reaction in skin can be used. A SOD/catalase mimetic that has catalase activity in vivo can also be used. A SOD/catalase mimetic that has catalase activity in a human subject can also be used. Accordingly, when manufacturing a SOD/catalase mimetic that is useful for the treatment of a disease to ensure that any components added to the formulation do not inhibit or modify the catalase activity of the active SOD/catalase mimetic.

Pharmaceutical formulations may be presented in unit dose forms containing a predetermined amount of active ingredient per unit dose. Such a unit may contain for example 1 μg to 10 μg, such as 0.01 mg to 1000 mg, or 0.1 mg to 250 mg, for e.g., a SOD/catalase mimetic of Structures I-XXXVI or any one of the compounds as shown in FIG. 5A through 7C, depending on the condition being treated, the route of administration and the age, weight and condition of the patient. In one embodiment, the amount of active agent can be from 0.01% to 5% by weight in the formulation.

Pharmaceutical formulations adapted for transferal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), p318 et seq. (1986).

Pharmaceutical formulations adapted for topical administration may be formulated as ointments, creams, suspensions, lotions, powders, solutions, pastes, gels, sprays, aerosols or oils.

For treatments of external tissues, for example mouth and skin, the formulations can be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

SOD/catalase mimetics may be formulated into a cosmetic base for topical application and/or for reducing oxidation of the cosmetic by molecular oxygen and oxyradicals. The pharmaceutical formulation used in the process according to any embodiment as described herein, may also comprise other functional ingredients. It will be apparent to the skilled artisan what functional ingredients are to be included and is dependent upon the symptoms of skin damage associated with oxidative skin damage that are being treated.

In one example, the formulation further comprises sun-block with an SPF rating to block UVB rays and/or compounds such as for example, titanium dioxide, zinc oxide and/or avobenzone, which helps protect against UVA rays.

In another example, the formulation further comprises anti-acne formulations such as for example, benzoyl peroxide, bacitracin and/or other generic anti-acne compounds.

In another example, the formulation further comprises anti-allergic agents such as for example, any known blocker of histamine release in skin.

In another example, the formulation further comprises a known compound that is a useful in an anti-ageing formulation, such as, but not limited to a matrix metalloproteinase (MMP) inhibitor, folic acid, creatine, revitol, hydroderm, ceramide c, hyaluronic acid, vitamin A, vitamin C, vitamin E, and glycolic compounds.

In another example, the formulation further comprises a known compound that is useful for wound-healing such as, but not limited to a MMP inhibitor, or any ligand or modulator of the signalling pathways of growth factors belonging to the TGF-β superfamily of ligands.

In another example, the formulation further comprises anti-inflammatory agents such as for example, alclometason, amcinonide, benzoyl peroxide, betamethasone, clobetasol, cortisone, hydrocortisone, desonide, desoximetasone, diflorasone, or any agents that treat symptoms associated with dermatitis, including psoriasis, and eczema that may be formulated with an anti-inflammatory agent in a cosmetic base for topical application for local prevention of inflammation and/or tissue damage consequent to inflammation.

A variety of steroidal and non-steroidal anti-inflammatory agents can be combined with the SOD/catalase mimetic. For example, steroidal anti-inflammatory agents, including but not limited to, corticosteroids such as hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednis one acetate, clocortelone, clescinolone, dichlorisone, difluprednate, flucloronide, flunisolide, fluoromethalone, fluperolone, flupreclnisolone, hydrocortisone valerate, hydrocortisone cyclopentylpropionate, hydrocortamate, eprednisone, paramethasone, prednisolone, prednisone, beclomethasone dipropionate, triamcinolone, and mixtures thereof may be used. In one embodiment, steroidal anti-inflammatory for use in the present methods is hydrocortisone.

Specific non-steroidal anti-inflammatory agents useful in the composition include, but are not limited to: piroxicam, isoxicam, tenoxicam, sudoxicam, CP-14,304, aspirin, disalcid, benorylate, trilisate, safapryn, solprin, diflunisal, fendosal, diclofenac, fenclofenac, indomethacin, sulindac, tolmetin, isoxepac, furofenac, tiopinac, zidometacin, aσemetacin, fentiazac, zomepirac, clidanac, oxepinac, felbinac, mefenamic, meclofenamic, flufenamic, niflumic, tolfenamic acids, ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, al inoprofen, tiaprofenic, phenylbutazone, oxyphenbutazone, feprazone, azapropazone, and trimethazone, among others.

Mixtures of these non-steroidal anti-inflammatory agents may also be employed, as well as the pharmaceutically-acceptable salts and esters of these agents. For example, etofenamate, a flufenamic acid derivative, is particularly useful for topical application.

Of the nonsteroidal anti-inflammatory agents, ibuprofen, naproxen, flufenamic acid, mefenamic acid, meclofenamic acid, piroxicam and felbinac can be used and ibuprofen, naproxen, and flufenamic acid can also be used.

Finally, so-called “natural” anti-inflammatory agents can be useful. For example, candelilla wax, alpha bisabolol, aloe vera, Manjistha (extracted from plants in the genus Rubia, particularly Rubia Cordifolia), and Guggul (extracted from plants in the genus Commiphora, particularly Commiphora Mukul), may be used.

The pharmaceutical/cosmetic formulations used in the process according to any embodiment describe herein formulated as solutions can include a pharmaceutically- or cosmetically-acceptable organic solvent. The terms “pharmaceutically-acceptable organic solvent” and “cosmetically-acceptable organic solvent” refer to an organic solvent which, in addition to being capable of having dispersed or dissolved therein the SOD/catalase mimetic, and optionally also an anti-inflammatory agent, also possesses acceptable safety (e.g. irritation and sensitization characteristics), as well as good aesthetic properties (e.g., does not feel greasy or tacky). One example of such a solvent is isopropanol. Examples of other suitable organic solvents include: propylene glycol, polyethylene glycol (200-600), polypropylene glycol (425-2025), glycerol, 1,2,4-butanetriol, sorbitol esters, 1,2,6-hexanetriol, ethanol, butanediol, water and mixtures thereof. These solutions contain from about 0.0001% to about 20%, in one embodiment from about 0.01% to about 1%, SOD/Catalase mimetic, from about 0.01% to about 5%, in another embodiment from about 0.5% to about 2% of an anti-inflammatory agent, and from about 80% to about 99%, in yet another embodiment from about 90% to about 98%, of an acceptable organic solvent. As used herein, “emollients” refer to materials used for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972), incorporated herein by reference, contains numerous examples of suitable materials. Examples of classes of useful emollients include the following:

1. Hydrocarbon oils and waxes. Examples include mineral oil, petrolatum, paraffin, ceresin, ozokerite, icrocrystalline wax, polyethylene, and perhydrosqualene.
2. Silicone oils, such as dimethyl polysiloxanes, methylphenyl polysiloxanes, water-soluble and alcohol-soluble silicone glycol copolymers.
3. Triglyceride esters, for example vegetable and animal fats and oils. Examples include castor oil, safflower oil, cottonseed oil, corn oil, olive oil, cod liver oil, almond oil, avocado oil, palm oil, sesame oil, and soybean oil.
4. Acetoglyceride esters, such as acetylated monoglycerides.
5. Ethoxylated glycerides, such as ethoxylated glyceryl monostearate.
6. Alkyl esters of fatty acids having 10 to 20 carbon atoms. Methyl, isopropyl, and butyl esters of fatty acids are particularly useful herein. Examples of other useful alkyl esters include hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, auryl lactate, myristyl lactate, and cetyl lactate.
7. Alkenyl esters of fatty acids having 10 to 20 carbon atoms. Examples include oleyl myristate, oleyl stearate, and oleyl oleate.
8. Fatty acids having 10 to 20 carbon atoms. Suitable examples include pelargonic, lauric, myristic, palmitic, stearic, isosstearic, hydroxystearic, oleic, linoleic, ricinoleic, arachidic, behenic, and erucic acids.
9. Fatty alcohols having 10 to 20 carbon atoms. Lauryl, myristyl, cetyl, hexadecyl, stearyl, isostearyl, hydroxystearyl, oleyl, ricinoleyl, behenyl, and erucyl alcohols, as well as 2-octyl dodecanol, are examples of satisfactory fatty alcohols.
10. Fatty alcohol ethers. Ethoxylated fatty alcohols of 10 to 20 carbon atoms include the lauryl, cetyl, stearyl, isostearyl, oelyl, and cholesterol alcohols having attached thereto from 1 to 50 ethylene oxide groups or 1 to 50 propylene oxide groups.
11. Ether-esters such as fatty acid esters of ethoxylated fatty alcohols.
12. Lanolin and derivatives. Lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases are illustrative of emollients derived from lanolin.
13. Polyhydric alcohols and polyether derivatives. Propylene glycol, dipropylene glycol, polypropylene glycols 2000 and 4000, polyoxyethylene polyoxypropylene glycols, polyoxypropylene polyoxyethylene glycols, glycerol, sorbitol, ethoxylated sorbitol, hydroxypropyl sorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol (2-methyl-2,4-pentanediol), 1,3-butylene glycol, 1,2,6-hexanetriol, ethohexadiol USP (2-ethyl-1,3-hexanediol), C15-C18 vicinal glycol, and polyoxypropylene derivatives of trimethylolpropane are examples of this class of materials.
14. Polyhydric alcohol esters. Ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters for use herein.
15. Wax esters such as beeswax, spermaceti, myristyl myristate, stearyl stearate.
16. Beeswax derivatives, e.g. polyoxyethylene sorbitol beeswax. These are reaction products of beeswax with ethoxylated sorbitol of varying ethylene oxide content, forming a mixture of ether-esters.
17. Vegetable waxes including carnauba and candelilla waxes.
18. Phospholipids, such as lecithin and derivatives.
19. Sterols. Cholesterol and cholesterol fatty acid esters are examples thereof
20. Amides such as fatty acid amides, ethoxylated fatty acid amides, solid fatty acid alkanolamides.

Particularly useful emollients which provide skin conditioning are glycerol, hexanetriol, butanetriol, lactic acid and its salts, urea, pyrrolidone carboxylic acid and its salts, amino acids, guanidine, diglycerol and triglycerol. In one embodiment, skin conditioning agents are the propoxylated glycerol derivatives.

Pharmaceutical formulations adapted for oral administration may be presented as discrete units such as capsules, soft gels, or tablets; powders or granules; solutions or suspensions in aqueous or non-aqueous liquids; edible foams or whips; or oil-in-water liquid emulsions or water-in-oil liquid emulsions. In one embodiment, oral formulations account for the relative lipophilicity of the SOD/catalase mimetics of Structures I-XXXVI or any one of the compounds as shown in FIG. 5A through 7C.

In general, formulations suitable for oral steroid compositions can be oral formulations for the SOD/catalase mimetics.

In one example, the oral formulation comprises an intragranular phase comprising an effective amount of a SOD/catalase mimetics and at least one carbohydrate alcohol and an aqueous binder. In one embodiment, the pharmaceutical formulation is substantially lactose-free. Some carbohydrate alcohols for such formulations can be selected from the group consisting of mannitol, maltitol, sorbitol, lactitol, erythritol and xylitol. In another embodiment, the carbohydrate alcohol is present at a concentration of about 15% to about 90%. In yet another embodiment, aqueous binder is selected from the group consisting of hydroxypropyl cellulose, hydroxypropyl methylcellulose, carboxymethyl cellulose sodium, polyvinyl pyrrolidones, starches, gelatins and povidones. A binder is generally present in the range of from about 1% to about 15%. The intragranular phase can also comprise one or more diluents, such as, for example, a diluent selected from the group consisting of microcrystalline cellulose, powdered cellulose, calcium phosphate-dibasic, calcium sulfate, dextrates, dextrins, alginates and dextrose excipients. Such diluents are also present in the range of about 15% to about 90%. The intragranular phase can also comprise one or more disintegrants, such as, for example, a disintegrant selected from the group consisting of a low substituted hydroxypropyl cellulose, carboxymethyl cellulose, calcium carboxymethylcellulose, sodium carboxymethyl cellulose, sodium starch glycollate, crospovidone, croscarmellose sodium, starch, crystalline cellulose, hydroxypropyl starch, and partially pregelatinized starch. A disintegrant is generally present in the range of from about 5% to about 20%. Such a formulation can also comprise one or more lubricants such as, for example, a lubricant selected from the group consisting of talc, magnesium stearate, stearic acid, hydrogenated vegetable oils, glyceryl behenate, polyethylene glycols and derivatives thereof, sodium lauryl sulphate and sodium stearyl fumarate. A lubricant is generally present in the range of from about 0.5% to about 5%. Such formulations are made into a tablet, capsule, or soft gel e.g., by a process comprising mixing a metallophorphyrin derivative and at least one carbohydrate alcohol to form a dry blend, wet granulating the dry blend with an aqueous binder so as to obtain an intragranular phase, and further formulating the resulting intragranular phase so as to provide the formulation. A tablet or capsule will be prepared to contain from 1 mg to 1000 mg, such as 2.5 mg to 250 mg of active ingredient per unit dose.

A liquid or semi-solid pharmaceutical formulation for oral administration e.g., a hard gel or soft gel capsule, may be prepared comprising:

(a) a first carrier component comprising from about 10% to about 99.99% by weight of a SOD/Catalase mimetic;
(b) an optional second carrier component comprising, when present, up to about 70% by weight of said;
(c) an optional emulsifying/solubilizing component comprising, when present, from about 0.01% to about 30% by weight of said SOD/Catalase mimetic;
(d) an optional anti-crystallization/solubilizing component comprising, when present, from about 0.01% to about 30% by weight of said SOD/Catalase mimetic; and
(e) an active pharmacological agent comprising from about 0.01% to about 80% of said SOD/catalase mimetics in anhydrous crystal form.

The first carrier component and optional second carrier component generally comprise, independently, one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, glycerol, sorbic acid, sorbitol, or polyethoxylated vegetable oil.

The emulsifying/solubilizing component generally comprises one or more of metallic alkyl sulfate, quaternary ammonium compounds, salts of fatty acids, sulfosuccinates, taurates, amino acids, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, polyoxyethylene fatty alcohol ether, fatty acid, polyethoxylated fatty acid ester, propylene glycol fatty acid ester, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil.

The anti-crystallization/solubilizing component, when present, generally comprises one or more of metallic alkyl sulfate, polyvinylpyrrolidone, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil.

Some formulations for oral delivery of a SOD/catalase mimetics used in the process according to any embodiment as described herein can account for its relative lipophilicity and ready absorption by the lining of the stomach and/or the intestine. By appropriate formulation of the compounds, their levels in body fluids such as plasma and urine can be enhanced, relative to their deposition in adipose tissues.

For example, a SOD/catalase mimetics is formulated with a hydrophobic polymer, in one embodiment a bioadhesive polymer and optionally can be encapsulated in or dispersed throughout a microparticle or nanoparticle. The bioadhesive polymer improves gastrointestinal retention via adherence of the formulation to the walls of the gastrointestinal tract. Suitable bioadhesive polymers include polylactic acid, polystyrene, poly(bis carboxy phenoxy propane-co-sebacic anhydride) (20:80) (poly (CCP:SA)), alginate (freshly prepared); and poly(fumaric anhydride-co-sebacic:anhydride (20:80) (poly (FA:SA)), types A (containing sudan red dye) and B (undyed). Other high-adhesion polymers include p(FA:SA) (50:50) and non-water-soluble polyacrylates and polyacrylamides. In one embodiment, bioadhesive polymers can be hydrophobic enough to be non-water-soluble, but contain a sufficient amount of exposed surface carboxyl groups to promote adhesion e.g., non-water-soluble polyacrylates and polymethacrylates; polymers of hydroxy acids, such as polylactide and polyglycolide; polyanhydrides; polyorthoesters; blends comprising these polymers; and copolymers comprising the monomers of these polymers. One biopolymers can be bioerodable, with molecular weights ranging from 1000 to 15,000 kDa, and in some cases 2000 to 5000 Da. Polyanhydrides e.g., polyadipic anhydride (“p(AA)”), polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different mole ratios, can be used.

Blends of hydrophilic polymers and bioadhesive hydrophobic polymers can also be employed. Suitable hydrophilic polymers include e.g., hydroxypropylmethylcellulose, hydroxypropylcellulose, carboxymethylcellulose, polyvinylalcohols, polyvinylpyrollidones, and polyethylene glycols.

Other mucoadhesive polymers include DOPA-maleic anhydride co polymer, isopthalic anhydride polymer, DOPA-methacrylate polymers, DOPA-cellulosic based polymers, and DOPA-acrylic acid polymers.

Excipients can be included in the dosage form e.g., to improve bioadhesion. Suitable excipients include solvents, co-solvents, emulsifiers, plasticizers, surfactants, thickeners, pH modifiers, emollients, antioxidants, and chelating agents, wetting agents, and water absorbing agents. The formulation may also include one or more additives, for example, dyes, colored pigments, pearlescent agents, deodorizers, and odor maskers.

The SOD/catalase mimetics may optionally be encapsulated or molecularly dispersed in polymers to reduce particle size and increase dissolution. The polymers may include polyesters such as poly(lactic acid) or P(LA), polycaprylactone, polylactide-coglycolide or P(LGA), poly hydroxybutyrate poly β-malic acid); polyanhydrides such as poly(adipic)anhydride or P(AA), poly(fumaric-co-sebacic)anhydride or P(FA:SA), poly(sebacic)anhydride or P(SA); cellulosic polymers such as ethylcellulose, cellulose acetate, cellulose acetate phthalate, etc; acrylate and methacrylate polymers such as Eudragit RS 100, RL 100, E100 PO, L100-55, L100, S100 (distributed by Rohm America) or other polymers commonly used for encapsulation for pharmaceutical purposes and known to those skilled in the art. Also suitable are hydrophobic polymers such as polyimides.

Blending or copolymerization sufficient to provide a certain amount of hydrophilic character can be useful to improve wettability of the materials. For example, about 5% to about 20% of monomers may be hydrophilic monomers. Hydrophilic polymers such as hydroxylpropylcellulose (HPC), hydroxpropylmethylcellulose (HPMC), carboxymethylcellulose (CMC) are commonly used for this purpose.

The formulation may be an “immediate release” formulation that releases at least 85% (wt/wt) of the active SOD/catalase mimetics within 60 minutes in vitro. Alternatively, the formulation is a “controlled release” formulation that releases drug more slowly than an immediate release formulation i.e., it takes longer than 60 minutes to release at least 85% (wt/wt) of the drug in vitro. To extend the time period for release, the ratio of SOD/catalase mimetics to polymer can be increased. Increased relative drug concentration is believed to have the effect of increasing the effective compound domain size within the polymer matrix thereby slowing dissolution. In the case of a polymer matrix containing certain types of hydrophobic polymers, the polymer will act as a mucoadhesive material and increase the retention time of the active compound in the gastrointestinal tract. Increased drug dissolution rates combined with the mucoadhesive properties of the polymer matrix increase uptake of the active compound and reduce differences found in the fed and fasted states for the compounds.

The oral formulations may be prepared using a pharmaceutically acceptable carrier composed of materials that are considered safe and effective and may be administered to an individual without causing undesirable biological side effects or unwanted interactions. Exemplary carrier include diluents, binders, lubricants, disintegrants, stabilizers, surfactants, colorants, and fillers.

Diluents or fillers increase the bulk of a solid dosage form so that a practical size is provided for compression of tablets or formation of beads and granules. Suitable diluents include, but are not limited to dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Dispersants include phosphate-buffered saline (PBS), saline, glucose, sodium lauryl sulfate (SLS), polyvinylpyrrolidone (PVP), polyethylene glycol (PEG), and hydroxypropylmethylcellulose (HPMC).

Binders may impart cohesive qualities to a solid dosage formulation, and thus ensure that a tablet, bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose (“HPMC”), microcrystalline cellulose (“MCC”), hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (PVP).

Lubricants may facilitate tablet manufacture. Examples of suitable lubricants include, but are not limited to, magnesium stearate, calcium stearate, stearic acid, glycerol behenate, polyethylene glycol, talc, and mineral oil.

Disintegrants may facilitate dosage form disintegration after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP.

Stabilizers may inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-00 monolaurate, polyoxyethylene monolaurate, polysorbates, polyoxyethylene octylphenylether, PEG-1000 cetyl ether, polyoxyethylene tridecyl ether, polypropylene glycol butyl ether, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

Pharmaceutical formulations adapted for parenteral administration include aqueous and non-aqueous sterile injection solutions which may contain the antioxidants as well as buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Formulation of a SOD/catalase mimetic used in the process according to any embodiment described herein, in an intravenous lipid emulsion or a surfactant micelle or polymeric micelle (see., e.g., Jones et al., Eur. J. Pharmaceutics Biopharmaceutics 48, 101-111, 1999; Torchilin J. Clin, release 73, 137-172, 2001; both of which are incorporated herein by reference) can be used.

Sustained release injectable formulations are produced e.g., by encapsulating the SOD/catalase mimetic in porous microparticles which comprise a pharmaceutical agent and a matrix material having a volume average diameter between about 1 μm and 150 μm, e.g., between about 5 μm and 25 μm diameter. In one embodiment, the porous microparticles have an average porosity between about 5% and 90% by volume. In one embodiment, the porous microparticles further comprise one or more surfactants, such as a phospholipid. The microparticles may be dispersed in a pharmaceutically acceptable aqueous or non-aqueous vehicle for injection. Suitable matrix materials for such formulations comprise a biocompatible synthetic polymer, a lipid, a hydrophobic molecule, or a combination thereof. For example, the synthetic polymer can comprise, for example, a polymer selected from the group consisting of poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers, derivatives and blends thereof. In one embodiment, the synthetic polymer comprises a poly(lactic acid), a poly(glycolic acid), a poly(lactic-co-glycolic acid), or a poly(lactide-co-glycolide).

The methods and compositions are further described by reference to the following non-limiting examples.

### Example 1

### Collection of Skin Lipid Samples with a Medical Device

A skin sample for use in the process is readily obtained by a health care practitioner, e.g., a dermatologist. In this example, the sample is obtained by a dermatologist when a subject suffering from symptoms of skin damage is being assessed by the dermatologist. The skin sample is obtained by ethanol wash of a region of a suitable region of the skin of the subject as defined herein. This may include a region of skin where symptoms of skin damage are present. A similar sample is obtained from a suitable region of the subject's skin where there are no apparent visible symptoms. The device as shown in FIGS. 1-4 is prepared by removing the protective membrane of the base, and the open-end of the base member is placed on the surface of a suitable region of the skin of a subject such that the open-end of the reservoir makes contact with the skin surface of the subject and makes a seal that prevents liquid leaking from the reservoir. Leakage is prevented by pressure exerted by the dermatologist taking the skin sample. Once the seal is made, the dermatologist then exerts pressure on the piston rod clearing the solution from the cylinder of the syringe portion and this removes the membrane seal at the opening of the cylinder allowing the solution to enter the reservoir of the base member and make contact with the suitable site of stratum corneum of the skin of the subject. The device is held in place for about 5 min. The solution is then removed from the reservoir by exerting a pulling force on the piston rod, in a similar fashion as obtaining a blood sample using a regular syringe, such that the solution enters the cylinder again and the device is removed from the subject. The solution is then transferred to a suitable vial for transferring off-site for the determination of the oxidative damage index. The oxidative damage index is determined as exemplified herein in Example 2.

### Example 2

### Measurement of Squalene and Squalene Monohydroperoxide in Ethanol Extracts from Human Skin Surface

Skin surface lipids are collected using a medical device according to the methods as described in Example 1, or via tape stripping as described in Giacomoni et al., 2000, IUBMB Life 49. Skin surface lipids are analyzed for squalene and sqOOH as described in detail elsewhere Maes, D., et al., 2000, Methods Enzymol., 319:612-622. Briefly, ethanol soluble lipids are collected by solvent extraction on the ventral forearm, forehead, or cheek, filtered through a 0.45 μm polytetrafluoroethylene (PTFE) filter, dried down in a SpeedVac vacuum concentrator (Savant, Holbrook, N.Y., U.S.A.), reconstituted in 200 μL ethanol and analyzed or stored at −20° C. until further analysis within 24 h. Squalene and sqOOH were separated from other lipids by reversed phase high-pressure liquid chromatography (HPLC) (Thermo Separation Products 1000 Series, San Jose, Calif., U.S.A.) as described earlier Maes et al., (supra) with modification to the following conditions: Column: Nucleosil octadecyl silane (ODS) 250×4.6 mm, 5 μm (Varian, Palo Alta, Calif., U.S.A.); mobile phase: 100% methanol (HPLC grade)—flow 1 ml min. Squalene is detected by a UV detector (Thermo Separation Products, UV2000, San Jose, Calif., U.S.A.) at 220 nm and is quantified against an external standard of squalene. SqOOH is detected by a second detector (Thermo Separation Products, UV6000, San Jose, Calif., U.S.A.) at 510 nm after on-line post-column reaction with a peroxide-specific reagent containing Fe(II) sulfate and 5-sulfosalicylic acid. Quantification is based on comparison to an external standard of sqOOH, prepared by oxidizing squalene in the presence of methylene blue and is titrated against a standardized solution of sodium thiosulfate.

The oxidative damage index in this example is defined as: sqOOH/(squalene+sqOOH)×100. The percent increase in baseline levels of lipid peroxides (sqOOH) is calculated by comparison to a non-affected site, i.e., wherein no symptoms of skin damage are visible. A positive value of percentage increase is indicative of the presence of skin damage.

### Example 3

### In Vitro Catalase Activity

The catalase activity of SOD/Catalase mimetics used in the process was determined as previously described by Doctrow et al., 2002, J. Med. Chem. 45, 4549-4558. Briefly, catalase activity was measured by incubating the sample compound with hydrogen peroxide and determining the amount of hydrogen peroxide remaining after a period of time using a colorimetric peroxidase-coupled assay method. 10 μM sample compound and 100 μM hydrogen peroxide in 40 mM sodium phosphate pH 7.4 were incubated together at ambient temperature in a multi-well plate. After the desired reaction period had elapsed, 20 μl of peroxidase/ABTS reagent was added (peroxidase/ABTS reagent contained 100 μl of 50 mM Na phosphate, pH 7.4, 1 mg horseradish peroxidase (1310 U/mg) and 1.6 g ABTS). After five minutes, absorbance at 750 nm was determined.

The amount of hydrogen peroxide remaining was calculated based on a standard curve. To compare rates of catalase reaction, the amount of hydrogen peroxide consumed at 20 minutes was determined.

The rates of the SOD/Catalase mimetics disclosed in U.S. Pat. Nos. 5,403,834; 5,827,880; 5,696,109; 5,834,509; 6,589,948; 7,122,537; 6,403,788; US Publication No. 2007/0123503; International Publication Nos: WO 94/13300; WO 96/40149; WO 96/40148; WO 2005/000854; WO 2008/033444; and related patent(s)/application(s) thereof were normalized to the rate for relative to C7 (EUK-8). Accordingly, the relative rates of the SOD/Catalase mimetics disclosed therein were calculated by the following formula:

(rate obtained for SOD/Catalase mimetic)/(rate obtained for C7 (EUK-8))×100%

Results are shown in Tables 1 to 4.

While this invention has been particularly shown and described with references to embodiments thereof, it will be understood by those skilled in the art that various changes in form and details may be made therein without departing from the spirit and scope of the invention as defined by the appended claims. Those skilled in the art will recognize, or be able to ascertain using no more than routine experimentation, many equivalents to the specific embodiments of the invention described specifically herein. Such equivalents are intended to be encompassed in the scope of the appended claims.

All patents, patent application, publications and other publicly available materials referenced herein are hereby incorporated by reference in their entirety.

What is claimed is:

1. A method of treating oxidative skin damage in a subject, comprising:
(i) obtaining a sample from a stratum corneum of a subject;
(ii) measuring the level of at least one oxidized substance in the sample;
(iii) comparing a detected level of the oxidized substance with a standard, whereby an elevated level of the oxidized substance is indicative of oxidative skin damage.

2. The method of claim 1 further comprising recommending a therapeutic regime for treatment of oxidative skin damage in the subject, wherein said recommendation comprises a recommendation to administer a pharmaceutical formulation comprising an amount of one or more specific synthetic SOD/catalase mimetics sufficient to treat the level of oxidative skin damage of the subject.

3. The method of claim 1 wherein the step of obtaining a sample further comprises extracting a lipid with a solvent comprising an organic solvent and a non-catalytic antioxidant.

4. The method of claim 1 wherein the step of obtaining the sample comprises collecting the sample using a non invasive collection apparatus.

5. The method of claim 4 wherein the step of collecting the sample further comprises contacting a region of skin with the apparatus, wherein the apparatus is in fluid communication with the skin region and comprises a solvent capable of extracting lipids from the stratum corneum.

6. The method of claim 1 wherein the step of measuring the level of at least one oxidized substance in the sample further comprises measuring a level of oxidized or peroxidized lipid in the sample.

7. The method of claim 1 wherein the step of measuring the level of at least one oxidized substance in the sample further comprises measuring a level of an oxidized phospholipid 1-palmitoyl-2-arachidonoyl-sn-glycero-3-phosphorylcholine (OxPAPC) or peroxidized squalene (sqOOH) in the sample.

8. The method of claim 1 wherein the step of measuring the level of at least one oxidized substance in the sample further comprises subjecting the extracted sample to chromatography or mass spectroscopy.

9. The method of claim 1 wherein the step of comparing the detected level further comprises comparing the detected level with a baseline level of oxidative damage derived from at least one healthy subject.

10. The method of claim 9 wherein the step of comparing the detected level further comprises calculating an oxidative damage index, wherein the oxidative damage index is a numerical ratio wherein a detected amount of an oxidized form of a substance present in the stratum corneum of any suitable part of the skin of a subject is measured and expressed relative to a detected amount of a non-oxidized form of that substance measured in the same skin sample of the subject.

11. The method of claim 10 wherein the oxidative damage index is calculated by determining relative amounts of oxidized and non-oxidized lipids in a sample of the stratum corneum of the subject according to the following formula:
oxidized lipid/(non-oxidized lipid+oxidized lipid)×100

12. The method of claim 10 wherein the oxidative damage index is calculated by determining relative amounts of oxidized and non-oxidized lipids in a sample of the stratum corneum of the subject according to the following formula:
peroxidated lipid/(non-peroxidated lipid+peroxidated lipid)×100

13. The method of claim 10 wherein the oxidative damage index is calculated by determining relative amounts of peroxidated squalene (sqOOH) and non-peroxidized squalene (sq) present in the sample according to the following formula:
sqOOH/(sq+sqOOH)×100

14. The method of claim 1 wherein the step of comparing the level of oxidative skin damage further comprises comparing the level of oxidative skin damage with a baseline level of oxidative damage derived from at least one healthy subject.

15. A method of diagnosing skin damage comprising:
obtaining a sample from the stratum corneum of a subject;
measuring the level of at least one oxidized substance and at least one non-oxidized substance in the sample; and evaluating a level of skin damage in a subject by:
(i) calculating an oxidative damage index of the sample; and
(ii) comparing the oxidative damage calculated at (i) to a baseline level of oxidative damage to skin of a healthy control subject, wherein a greater oxidative damage than baseline is indicative of the presence of skin damage in the skin of said subject.

16. The method of claim 15 wherein the step of obtaining the sample comprises collecting the sample using a non invasive collection apparatus.

17. The method of claim 16 wherein the step of collecting the sample further comprises contacting a region of skin with the apparatus, wherein the apparatus is in fluid communication with the skin region and comprises a solvent capable of extracting a substance from the skin.

18. The method of claim 15 wherein the oxidative damage index is a numerical ratio wherein a detected amount of an oxidized form of a substance present in the stratum corneum of any suitable part of the skin of a subject is measured and expressed relative to a detected amount of a non-oxidized form of that substance measured in the same skin sample of the subject.

19. The method of claim 18 wherein the oxidative damage index is calculated by determining relative amounts of oxidized and non-oxidized lipids in a sample of the stratum corneum of the subject according to the following formula:
oxidized lipid/(non-oxidized lipid+oxidized lipid)×100

20. The method of claim 18 wherein the oxidative damage index is calculated by determining relative amounts of oxidized and non-oxidized lipids in a sample of the stratum corneum of the subject according to the following formula:
peroxidated lipid/(non-peroxidated lipid+peroxidated lipid)×100

21. The method of claim 18 wherein the oxidative damage index is calculated by determining relative amounts of peroxidated squalene (sqOOH) and non-peroxidized squalene (sq) present in the sample according to the following formula:
sqOOH/(sq+sqOOH)×100

22. The method of claim 18 wherein the oxidative damage index is calculated by determining relative amounts of the oxidation product of phospholipid 1-palmitoyl-2-arachidonoyl-sn-glycero-3-phosphorylcholine (OxPAPC) and non-peroxidized phospholipid 1-palmitoyl-2-arachidonoyl-sn-glycero-3-phosphorylcholine (PAPC) present in the sample according to the following formula:
OxPAPC/(PAPC+OxPAPC)×100

23. The method of claim 15 wherein the step of measuring the level of at least one oxidized substance in the sample further comprises subjecting the extracted sample to chromatography or mass spectroscopy.

24. The method of claim 15 wherein the step of comparing the oxidative damage is performed by a programmed computer comprising a processor and a lookup table of one or more baseline values stored in memory.

25. The method of claim 15 further comprising recommending a therapeutic regime for treatment of oxidative skin damage in the subject, wherein said recommendation comprises a recommendation to administer a pharmaceutical formulation comprising an amount of one or more specific synthetic SOD/catalase mimetics sufficient to treat the level of oxidative skin damage of the subject.

26. The method of claim 25 wherein the therapeutic regime comprises deliverying the pharmaceutical formulation comprising an effective amount of a synthetic SOD/catalase mimetic to the subject's skin.

27. The method of claim 26 wherein the synthetic SOD/catalase mimetic is a salen-manganese complex such as salen-Mn(III) complex having a structure according to Structure I: 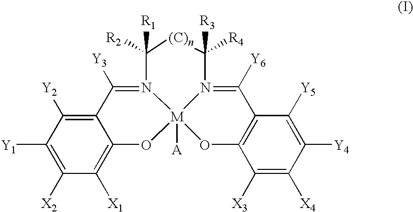
wherein M is a transition metal ion;
A can be an axial ligand composed of a halide, acetate, acetyl, acetoxy, ethoxy, formate, formyl, methoxy, PF₆, triflate, tosylate, A can be an oxygen atom bound to the transition metal (M), and A can be Cl, Br, F, MeO and OAc;
n can be 0, 1, 2, and 6;
X₁ X₂, X₃ and X₄ can be hydrogen, silyls, aryls, arylalkyls, primary alkyls, secondary alkyls, tertiary alkyls, alkoxys, aryloxys, aminos, quaternary amines, heteroatoms, F, Cl, Br, OAc, OMe, OH, and H;
Y₁, Y₂, Y₃, Y₄, Y₅, and Y₆ can be hydrogen, halides, alkyls, aryls, arylalkyls, silyl groups, aminos, aryls bearing heteroatoms, aryloxys and alkoxys; and
R, R₂, R₃ and R₄ can be H, CH₃, C₂H₅, C₆H₅, O-benzyl, primary alkyls, fatty acid esters, substituted alkoxyaryls, heteroatom-bearing aromatic groups, arylalkyls, secondary alkyls, and tertiary alkyls.

28. The method of claim 26 wherein the synthetic SOD/catalase mimetic is a salen-manganese complex having a structure according to any of the structures of compounds C1, C4, C6, C7, C9, C10, C11, C12, C15, C17, C20, C22, C23, C25, C27, C28, C29, C30, C31-C94 as shown in Figures or the Structures X-XXII as shown in FIGS. 5D through 5I.

29. The method of claim 26 wherein the synthetic SOD/catalase mimetic is a metalloporphyrin having a structure according to any of the structures XXVI-XXXII as shown in FIG. 5K-5O.

30. The method of claim 26 wherein the synthetic SOD/catalase mimetic is a cyclic salen-metal compound having a structure according to any of the structures C101-C155 as shown in FIGS. 6AA-6AQ.

31. The method of claim 26 wherein the synthetic SOD/catalase mimetic is an orally bioavailable water soluble metalloporphyrin derivative having a structure according to Structure XXXIV, Structure XXXV, Structure XXXVI as shown in FIGS. 5P-5Q or any of the compounds as shown in FIGS. 7A-7C.

32. An apparatus for diagnosing a skin condition comprising:
a skin contacting base member defining a reservoir adapted for fluid communication with a region of skin;
a source of solvent suitable for extracting a substance from the skin region; and
at least one channel for introducing the solvent into the reservoir when the reservoir is in fluid communication with the skin region and for withdrawing the solvent after it has extracted the substance from the skin.

33. The apparatus of claim 32 wherein the skin contacting base member comprises a device selected from the group of scrapers, cotton swaps, adhesive tape and solvent carrying towelette.

34. The apparatus of claim 32 wherein the base member further comprises a skin-contacting lip for maintaining contact without leakage from the reservoir when the based member is pressed against the skin and solvent is introduced into the chamber.

35. The apparatus of claim 32 wherein the solvent source comprises a releasable seal preventing introduction of the solvent into the reservoir until the seal is released by a user.

36. The apparatus of claim 32 wherein one channel serves for both introducing and removing the solvent.

37. The apparatus of claim 32 further comprising a syringe for introducing and withdrawing the solvent via the channel.

38. The apparatus of claim 32 further comprising at least two channels, one of which is adapted to introduce the solvent and the other adapted to withdraw the solvent.

39. The apparatus of claim 32 wherein the solvent comprises an organic solvent and a non-catalytic antioxidant.

40. The apparatus of claim 32 wherein the solvent comprises an alcohol-based solvent and a non-catalytic antioxidant.

41. The apparatus of claim 32 wherein the apparatus further comprises an indicator reagent that binds to one or more oxidized substances present in the sample.

42. The apparatus of claim 32 further comprises a collection chamber for storing the extracted substance and configured for injection of the sample into a chromatography apparatus or mass spectrometer.

43. The steps, features, integers, compositions and/or compounds disclosed herein or indicated in the specification of this application individually or collectively, and any and all combinations of two or more of said steps or features.
